SEQUENCE LISTING (72 sequences)

SEQ ID NO: 1 - DNA, 375 bp, Homo Sapiens
  CDS (1)..(375): 20A10-VH variable heavy chain (VH) sequence; 20A10 IgG4, kappa
caggtgcagctggtgcaatctggggctgaggtgaagaagcctgggtcctcagtgaaggtctcctgcaagacttctggaggcaccttcagcacctctactctcagctgggtgcgacaggcccctggacagggtcttgagtggctgggaggaatgatccctatccttagtagaacaacgtacgcgcagaagtttcagggcagactcacgattaccgcggacgaacccacgagcacgtcctacatggaactgagcagcctgaaatctgaggacacggccgtctattactgtgcgaccgagggggaagtttctgccgcgcacaactactactacggaatggacgtctggggccaagggaccacggtcaccgtctcctca

SEQ ID NO: 2 - protein, 125 aa, Homo Sapiens
QVQLVQSGAEVKKPGSSVKVSCKTSGGTFSTSTLSWVRQAPGQGLEWLGGMIPILSRTTYAQKFQGRLTITADEPTSTSYMELSSLKSEDTAVYYCATEGEVSAAHNYYYGMDVWGQGTTVTVSS

SEQ ID NO: 3 - DNA, 336 bp, Homo Sapiens
  CDS (1)..(336): 20A10-VL variable light chain (VL) sequence; kappa type
gatattgtgctgactcagtctccactctacctgcccgtcacccctggagagccggcctccatctcctgcaggtctagtcagagcctcctgcatggaaatggacacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaactctacatactgtattcactttcggccctgggaccagagtggatatcaaa

SEQ ID NO: 4 - protein, 112 aa, Homo Sapiens
DIVLTQSPLYLPVTPGEPASISCRSSQSLLHGNGHNYLDWYLQKPGQSPQLLIYLGSNRASGVPDRFSGSGSGTDFTLKISRVEAEDVGVYYCMQTLHTVFTFGPGTRVDIK

SEQ ID NO: 5 - DNA, 981 bp, Homo Sapiens
  CDS (1)..(981): 20A10-CH constant heavy chain (CH) sequence
gcttccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 6 - protein, 327 aa, Homo Sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - DNA, 321 bp, Homo Sapiens
  CDS (1)..(321): 20A10-CL constant light chain (CL) sequence
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 8 - protein, 107 aa, Homo Sapiens [sequence omitted]

SEQ ID NO: 9 - DNA, 360 bp, Homo Sapiens
  CDS (1)..(360): 2A11-VH variable heavy chain (VH) sequence; 2A11 IgG1, lambda
caggtgcatctggttcagtctggggctgaggtgaagaagcctgggtcctcggtgaaggtctcctgcaaggcttctggaggcaccttcaacaattttcccatgagttgggtgcgacaggcccctggacgagggcttgagtggatggcagggatcatccctgtatttggttcggcaaactatgcacagcagtttcacggcagagtcaccattagcgcggacatatccacgagcacggtgtccatggaactgaatgacttgaaatctgaggacacgggcgtttattattgtgcgattagtggagatgaggacaactggatcatgaacttttggggccagggaaccctggtcaccgtctcctca

SEQ ID NO: 10 - protein, 120 aa, Homo Sapiens
QVHLVQSGAEVKKPGSSVKVSCKASGGTFNNFPMSWVRQAPGRGLEWMAGIIPVFGSANYAQQFHGRVTISADISTSTVSMELNDLKSEDTGVYYCAISGDEDNWIMNFWGQGTLVTVSS

SEQ ID NO: 11 - DNA, 378 bp, Homo Sapiens
  CDS (1)..(360): 2A11-VL variable light chain (VL) sequence; lambda type
cagtctttgctgacgcagccgccctcagtgtctggggccccagggcagagagtcaccatctcctgcactgggagcagctccaacatcggggcaggttatgatgtgcactggtaccagcaagttccagggacagcccccaaactcctcatctatggaaacagaaatcggccctcaggggtccctgaccgattctctggctccaagtctggcacgtcagcctccctggccatcactgggctccaggctgaggatgaggctgattattactgccagtcctatgacagcagcctgagtggtcatgcggtgttcggcggagggaccaaggtgaccgtcctacgtcagcccaaggctgccccctcggtcactctgttcccgccc

SEQ ID NO: 12 - protein, 120 aa, Homo Sapiens
QSLLTQPPSVSGAPGQRVTISCTGSSSNIGAGYDVHWYQQVPGTAPKLLIYGNRNRPSGVPDRFSGSKSGTSASLAITGLQAEDEADYYCQSYDSSLSGHAVFGGGTKVTVLRQPKAAPS

SEQ ID NO: 13 - DNA, 990 bp, Homo Sapiens
  CDS (1)..(990): 2A11-CH constant heavy chain (CH) sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 14 - protein, 330 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 276 bp, Homo Sapiens
  CDS (1)..(276): 2A11-CL constant light chain (CL) sequence
tcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacagaagctacagctgccaggtcacgcatgaagggagcaccgtggagaagacagtggcccctacagaatgttca

SEQ ID NO: 16 - protein, 92 aa, Homo Sapiens
SSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - DNA, 363 bp, Homo Sapiens
  CDS (1)..(363): 7D1-VH variable heavy chain (VH) sequence; 7D1 IgG1, lambda
gaagtgcaactggtggcgtctgggggagacttcatacagcctggcaggtccctgagactctcctgtgcagcctctggattcaagtttgatgattatgccatgttctgggtccggcaagttccagggaggggcctggagtgggtctcagggattagttggagtagtgataagatggcctatgtggactctgtgaagggccgctttaccatctccagagacaacgccaagaacaccctctatttgcaaatgaacaatctgagacctgacgacacggccttctactactgtgcaaagggcggatttagcagtgtctggaactactttgacttctggggccagggaaccctggtcaccgtctcctca

SEQ ID NO: 18 - protein, 121 aa, Homo Sapiens
EVQLVASGGDFIQPGRSLRLSCAASGFKFDDYAMFWVRQVPGRGLEWVSGISWSSDKMAYVDSVKGRFTISRDNAKNTLYLQMNNLRPDDTAFYYCAKGGFSSVWNYFDFWGQGTLVTVSS

SEQ ID NO: 19 - DNA, 330 bp, Homo Sapiens
  CDS (1)..(330): 7D1-VL variable light chain (VL) sequence; lambda type
cagtctgttctgactcaaccaccctcagcgtctgggacccccgggcagacagtcatcatctcttgttctggaaccacctccaacatcggcagtaatactgtaagctggtaccggcaactcccaggggcggcccccaaactcctcatctttacttataatcagcggccctcaggggtccctgaccgattctctggctccaagtctggcacctctgcctccctggccatcagtggcctccagtctgacgatgaggctgattattactgtgccgtgtgggatgacgccctgggtggttgggtgttcggcggagggaccaagctgaccgtccta

SEQ ID NO: 20 - protein, 110 aa, Homo Sapiens
QSVLTQPPSASGTPGQTVIISCSGTTSNIGSNTVSWYRQLPGAAPKLLIFTYNQRPSGVPDRFSGSKSGTSASLAISGLQSDDEADYYCAVWDDALGGWVFGGGTKLTVL

SEQ ID NO: 21 - DNA, 990 bp, Homo Sapiens; identical to SEQ ID NO: 13
  CDS (1)..(990): 7D1-CH constant heavy chain (CH) sequence

SEQ ID NO: 22 - protein, 330 aa, Homo Sapiens; identical to SEQ ID NO: 14

SEQ ID NO: 23 - DNA, 318 bp, Homo Sapiens
  CDS (1)..(318): 7D1-CL constant light chain (CL) sequence
ggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctacctgagcctgacgcctgagcagtggaagtcccacaaaagctacagctgccaggtcacgcatgaagggagcaccgtggagaagacagtggcccctacagaatgttca

SEQ ID NO: 24 - protein, 106 aa, Homo Sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 25 - DNA, 366 bp, Homo Sapiens
  CDS (1)..(366): 6E11-VH variable heavy chain (VH) sequence; 6E11 IgG1, kappa
caggtgcagctggtgcagtctggggcagaggtgaagaagcctgggtcctcagtgaaggtctcctgcaaggcctctggcggcaccttccgcaactatgcgatcaactgggtgcggcaggccccgagacaaggacttgagtggatgggaagcctcgtccctatgtttacctcaccaacgtacgcacagaagtttcggggcagagtcacgataagcgcggacgagtccacggccacatcctccatggaactgaccagcctgacatctgaagacacggccgtctacttctgttctgcagatggctacaagggcggcctcttttacggtatgaatgtctggggccaagggaccacggtcaccgtctcttca

SEQ ID NO: 26 - protein, 122 aa, Homo Sapiens
QVQLVQSGAEVKKPGSSVKVSCKASGGTFRNYAINWVRQAPRQGLEWMGSLVPMFTSPTYAQKFRGRVTISADESTATSSMELTSLTSEDTAVYFCSADGYKGGLFYGMNVWGQGTTVTVSS

SEQ ID NO: 27 - DNA, 339 bp, Homo Sapiens
  CDS (1)..(339): 6E11-VL variable light chain (VL) sequence; kappa type
gacatcgtgttgacccagtctccagaatccctggctgtgtctctgggcgagagggccaccatcaactgcaagtctagccagagtgttttatacagctccaacagtaagaactacttagcttggttccagcagaaacctggacagcctcctaaacttctcatttattgggcatctacccgggaatccggggtccctgatcgcttcagtggcagcgggtctgggacagatttcactctcaccatcaccagcctgcaggctgaagatgtggcagtttattactgtcaccaatatcatactctacctcgaacgttcggccacgggaccaaggttgagatcaaa

SEQ ID NO: 28 - protein, 113 aa, Homo Sapiens
DIVLTQSPESLAVSLGERATINCKSSQSVLYSSNSKNYLAWFQQKPGQPPKLLIYWASTRESGVPDRFSGSGSGTDFTLTITSLQAEDVAVYYCHQYHTLPRTFGHGTKVEIK

SEQ ID NO: 29 - DNA, 987 bp, Homo Sapiens
  CDS (1)..(987): 6E11-CH constant heavy chain (CH) sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 329 aa, Homo Sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 321 bp, Homo Sapiens; identical to SEQ ID NO: 7
  CDS (1)..(321): 6E11-CL constant light chain (CL) sequence

SEQ ID NO: 32 - protein, 107 aa, Homo Sapiens [sequence omitted]

SEQ ID NO: 33 - DNA, 37 bp, Artificial Sequence
  IL20 specific 26bp, flanked with EcoRI site
tttggatcctactgaagacactcaatttgggaagctg

SEQ ID NO: 34 - DNA, 37 bp, Artificial Sequence
  IL20 specific 26bp, flanked with NotI site
tttgcggccgcctattctgtctcctccatccattgca

SEQ ID NO: 35 - DNA, 372 bp, Homo Sapiens
  CDS (1)..(372): 26B9-VH variable heavy chain (VH) sequence; 26B9 IgG1, kappa
cagatactactgcaggagtcgggcccaggactggtgaagcccacggagaccctgtccctcacctgtagtgtctctggtgactccatcagtgatagtagtcactactgggcctggattcgccagcccccagggaagggaccagagtggattggcagtgtctattttagttcgatgacccactacaacccgtccctcaaaagtcgcgtcagcatctccgttgacaagcccaagaaccagttctccttaaaagtgacctctgtgactgtcgccgacacggccacatattactgtgcgagacaagcccttgcccgagtcggagccatgaattggttcgacccctggggccagggatctctggtcacagtctcctca

SEQ ID NO: 36 - protein, 124 aa, Homo Sapiens
QILLQESGPGLVKPTETLSLTCSVSGDSISDSSHYWAWIRQPPGKGPEWIGSVYFSSMTHYNPSLKSRVSISVDKPKNQFSLKVTSVTVADTATYYCARQALARVGAMNWFDPWGQGSLVTVSS

SEQ ID NO: 37 - DNA, 339 bp, Homo Sapiens
  CDS (1)..(339): 26B9-VL variable light chain (VL) sequence, kappa type
gacatcataatgacccagtctccagactccctgcctgtgtctctgggcgagggggtcaccatcaactgcaagtccagccagagcgtctttttcacctccagtaataagagttgtttagcttggtatcagcagaagccaggaaagtctcccaaattgctcatttactgggcatcaacccgccaatccggggtccctgaccgattcagaggcagcgggtctgggacagatttctctctcaccatcaccagtctgcaggctgaagatgtggctgtttatttctgtcagcagtgtcagacatcccctcccactttcggcggagggaccaggttggagatcaaa

SEQ ID NO: 38 - protein, 113 aa, Homo Sapiens
DIIMTQSPDSLPVSLGEGVTINCKSSQSVFFTSSNKSCLAWYQQKPGKSPKLLIYWASTRQSGVPDRFRGSGSGTDFSLTITSLQAEDVAVYFCQQCQTSPPTFGGGTRLEIK

SEQ ID NO: 39 - DNA, 993 bp, Homo Sapiens
  CDS (1)..(993): 26B9-CH constant heavy chain (CH) sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 40 - protein, 330 aa, Homo Sapiens; identical to SEQ ID NO: 14

SEQ ID NO: 41 - DNA, 324 bp, Homo Sapiens
  CDS (1)..(324): 26B9-CL constant kappa chain (CL) sequence
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 42 - protein, 107 aa, Homo Sapiens [sequence omitted]

SEQ ID NO: 43 - DNA, 372 bp, Homo Sapiens
  CDS (1)..(372): 31B4-VH variable heavy chain (VH) sequence; 31B4 IgG1, kappa
cagatacagctgcaggagtcgggcccaggactggtgaggcccacggagaccctgtccctcacttgtagtgtctctggtgactccatcagtcagagtagtcattactgggcctggattcgccagcccccagggaagggaccagaatggattggcagtgtctattttagctcgatgacccactacaacccgtccctcacaagtcgcgtcagcatctccattgacaaggccatgaataagttctccttaaaagtgacctctgtgactgtcgccgacacggccacatattactgtgcgagacaggcccttgcccgagtcggagccatgaattggttcgacccctggggccagggatctctggtcacagtctcctca

SEQ ID NO: 44 - protein, 124 aa, Homo Sapiens
QIQLQESGPGLVRPTETLSLTCSVSGDSISQSSHYWAWIRQPPGKGPEWIGSVYFSSMTHYNPSLTSRVSISIDKAMNKFSLKVTSVTVADTATYYCARQALARVGAMNWFDPWGQGSLVTVSS

SEQ ID NO: 45 - DNA, 339 bp, Homo Sapiens
  CDS (1)..(339): 31B4-VL variable light chain (VL) sequence, kappa type
gacatcataatgacccagtctccagagtccctgcctgtgtctctgggcgagggggtcaccatcaactgcaagtccagccagagcgtctttttcacctccagtaataggagttgtttagcttggtatcagcagaagccaggacagtctcccaaattgctcatttactgggcatcaacccgccaatccggggtccctgaccgattcacaggcagcgggtctgggacagatttctctctcaccatcgccggtctgcaggttgaagatgtggctgtttatttctgtcagcagtgtcacgcatcccctcccactttcggcggcgggaccaggttggagctcaga

SEQ ID NO: 46 - protein, 113 aa, Homo Sapiens
DIIMTQSPESLPVSLGEGVTINCKSSQSVFFTSSNRSCLAWYQQKPGQSPKLLIYWASTRQSGVPDRFTGSGSGTDFSLTIAGLQVEDVAVYFCQQCHASPPTFGGGTRLELR

SEQ ID NO: 47 - DNA, 993 bp, Homo Sapiens; identical to SEQ ID NO: 39
  CDS (1)..(993): 31B4-CH constant heavy chain (CH) sequence

SEQ ID NO: 48 - protein, 330 aa, Homo Sapiens; identical to SEQ ID NO: 14

SEQ ID NO: 49 - DNA, 324 bp, Homo Sapiens; identical to SEQ ID NO: 41
  CDS (1)..(324): 31B4-CL constant kappa chain (CL) sequence

SEQ ID NO: 50 - protein, 107 aa, Homo Sapiens [sequence omitted]

SEQ ID NO: 51 - DNA, 372 bp, Homo Sapiens
  CDS (1)..(372): 8H1-VH variable heavy chain (VH) sequence; 8H1 kappa
caggtgcagctggtgcagtctggggctgaggtgaagaagcctggggcctcagtgaaggtctcctgcaaggcttctggacagaccttcaccagtgatgatatcaactgggtgcgacaggcccctggacaggggctagagtggatgggatggaggaaccctaacactcaggacacgggctatgcacagaagttccacggcagactcaccttgaccagcaacagttccataagtacatcctatctggagttgagcggcctgagatctgaggacacggccgtgtattactgtgcgagagcggggacttcgaccttgaccggccactacttcgctttgggggtctggggccaggggaccacggtcatcgtctcctca

SEQ ID NO: 52 - protein, 124 aa, Homo Sapiens
QVQLVQSGAEVKKPGASVKVSCKASGQTFTSDDINWVRQAPGQGLEWMGWRNPNTQDTGYAQKFHGRLTLTSNSSISTSYLELSGLRSEDTAVYYCARAGTSTLTGHYFALGVWGQGTTVIVSS

SEQ ID NO: 53 - DNA, 321 bp, Homo Sapiens
  CDS (1)..(321): 8H1-VL variable light chain (VL) sequence, kappa type
gacatccagctgacccagtctccatcctccctgtctgcatctgtaggagacagagtcaccatcacttgtcaggcgactcaggatattagcaaatatttaaattggtatcagcagaaaccagggaaagtccctaaactcctgatctacgaaacatccaatttggaagtaggggtcccatcaaggttcagtggaagtgggtctgggacacattttactctcaccatcagcagcctgcaggctgaagattttgcaacatattactgtcaacagtatgagaatttcccgttcactttcggcggagggaccaaggtggagatcaaa

SEQ ID NO: 54 - protein, 107 aa, Homo Sapiens
DIQLTQSPSSLSASVGDRVTITCQATQDISKYLNWYQQKPGKVPKLLIYETSNLEVGVPSRFSGSGSGTHFTLTISSLQAEDFATYYCQQYENFPFTFGGGTKVEIK

SEQ ID NO: 55 - DNA, 990 bp, Homo Sapiens; identical to SEQ ID NO: 13
  CDS (1)..(990): 8H1-CH constant heavy chain (CH) sequence

SEQ ID NO: 56 - protein, 330 aa, Homo Sapiens; identical to SEQ ID NO: 14

SEQ ID NO: 57 - DNA, 324 bp, Homo Sapiens; identical to SEQ ID NO: 41
  CDS (1)..(324): 8H1-CL constant kappa chain (CL) sequence

SEQ ID NO: 58 - protein, 107 aa, Homo Sapiens [sequence omitted]

SEQ ID NO: 59 - DNA, 378 bp, Homo Sapiens
  CDS (1)..(378): 19D11-VH variable heavy chain (VH) sequence; 19D11 IgG1, kappa
gaggtgcagctgttggagtctggggctgaggtgaagaggcctgggtcgtcggtgagggtctcctgcagggcttctggagacaccttcagcagttaccctatcagttgggtgcgacaggcccctggacaaggccttgagtggatgggaaggatcctccctgcccttggtgtcacaaactacgctcagaacttccggggcagaatcacgattaccgcggacaagtcgcccctcacagcctacttggaactgagtagcctcagatttgaggacacggccgtgtattactgtgcgagtcccagtgcggacataattccttcgattttggggacgaccctctttgccttctggggccagggaagcctggtcaccgtctcctca

SEQ ID NO: 60 - protein, 126 aa, Homo Sapiens
EVQLLESGAEVKRPGSSVRVSCRASGDTFSSYPISWVRQAPGQGLEWMGRILPALGVTNYAQNFRGRITITADKSPLTAYLELSSLRFEDTAVYYCASPSADIIPSILGTTLFAFWGQGSLVTVSS

SEQ ID NO: 61 - DNA, 330 bp, Homo Sapiens
  CDS (1)..(330): 19D11-VL variable light chain (VL) sequence, kappa type
gaaattgtgttgacgcagtctccaggcaccctgtctctgtctccgggggaaggggccaccctctcctgcagggccagtcagaatgttagcagacactacttaacctggtaccagcagaaacctggccagtctccccggctcctcatctatggtggctccagcagggccactggcgtcccagacaggttcagtggcggtgggtctgggacagacttcactctcaccatcagcaggctggagcctgaagactttgcagtgttttactgccagagctatcatagcccacctcctgtgtacactttcggccaggggaccaaggtggagatcaaa

SEQ ID NO: 62 - protein, 110 aa, Homo Sapiens
EIVLTQSPGTLSLSPGEGATLSCRASQNVSRHYLTWYQQKPGQSPRLLIYGGSSRATGVPDRFSGGGSGTDFTLTISRLEPEDFAVFYCQSYHSPPPVYTFGQGTKVEIK

SEQ ID NO: 63 - DNA, 993 bp, Homo Sapiens
  CDS (1)..(993): 19D11-CL constant kappa chain (CL) sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaanancctctccctgtccccgggtaaatga

SEQ ID NO: 64 - protein, 330 aa, Homo Sapiens
  misc_feature (322)..(322): The ′Xaa′ at location 322 stands for Lys, or Asn.
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQXXLSLSPGK

SEQ ID NO: 65 - DNA, 324 bp, Homo Sapiens; identical to SEQ ID NO: 41
  CDS (1)..(324): 19D11-CL constant kappa chain (CL) sequence

SEQ ID NO: 66 - protein, 107 aa, Homo Sapiens [sequence omitted]

SEQ ID NO: 67 - DNA, 183 bp, Artificial Sequence
  Sequence of transmembrane region region
gaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttag

SEQ ID NO: 68 - protein, 60 aa, Artificial Sequence
  Synthetic Construct
EQKLISEEDLNAVGQDTQEVIVVPHSLPFKVVVISAILALVVLTIISLIILIMLWQKKPR

SEQ ID NO: 69 - protein, 18 aa, Artificial Sequence
  Peptide fragment of human IL-20
PDHYTLRKISSLANSFLT

SEQ ID NO: 70 - protein, 15 aa, Artificial Sequence
  Peptide fragment of human IL-20
DHYTLRKISSLANSF

SEQ ID NO: 71 - protein, 18 aa, Artificial Sequence
  Peptide fragment of human IL-20
NYQTPDHYTLRKISSLAN

SEQ ID NO: 72 - protein, 17 aa, Artificial Sequence
  Peptide used for epitope characterization by alanine scan
PDHYTLRKISSLANSFL

(19) United States
(12) Patent Application Publication
(10) Pub. No.: US 2017/0189524 A1
(43) Pub. Date: Jul. 6, 2017

(54) HUMAN-DERIVED ANTI-HUMAN IL-20 ANTIBODIES AND ASSAY FOR THE IDENTIFICATION OF ANTI-CYTOKINE ANTIBODIES

(71) Applicant: IMMUNOQURE AG, Dusseldorf (DE)

(72) Inventors: Steffen MEYER, Munich (DE); Philip VLAICU, Alling (DE); Annalisa MACAGNO, Schlieren (CH); Part PETERSON, Tallinn (EE); Kai KISAND, Tartu (EE)

(21) Appl. No.: 15/321,842

(22) PCT Filed: Jul. 3, 2015
(86) PCT No.: PCT/EP2015/065244

§ 371 Date: Dec. 23, 2016

(30) Foreign Application Priority Data
Jul. 3, 2014 (EP) 14175585.0
Jul. 3, 2014 (EP) 14175673.4

(51) Int. Cl. A61K 39/395; G01N 33/50; G01N 33/68; C07K 16/24

(52) U.S. Cl. CPC A61K 39/3955; C07K 16/244; G01N 33/5008; G01N 33/6863; C07K 2317/565; C07K 2317/33; G01N 2500/10; C07K 2317/567; C07K 2317/76; C07K 2317/21

(57) ABSTRACT

Provided are novel interleukin-20 (IL-20) binding molecules of human origin, particularly human-derived anti-human IL-20 antibodies as well as IL-20 binding fragments, derivatives and biotechnological derivatives thereof. In addition, pharmaceutical compositions, kits and methods for use in diagnosis and therapy are described. In addition, a cellular enzyme-linked ligand binding assay is described for isolating antibodies and biotechnological derivatives thereof for pharmaceutical use, in particular recombinant human-derived anti-human cytokine antibodies.

### FIELD OF THE INVENTION

The present invention generally relates to novel molecules binding Interleukin-20 (IL-20) of mammal, preferably human origin, particularly human monoclonal antibodies as well as fragments, derivatives and variants thereof. In particular, the present invention relates to recombinant human patient-derived anti-IL-20 antibodies as well as fragments, derivatives and synthetic or biotechnological variants thereof that recognize IL-20. In addition, compositions comprising such binding molecules, antibodies and mimics thereof useful in the treatment and diagnosis of disorders are described. Furthermore, the present invention relates to the anti-IL-20 antibodies and mentioned equivalents thereof for use in immunotherapy as well as targets in the therapeutic intervention of autoimmune and autoinflammatory disorders as well as malignancies, such as various forms of arthritis, vascular inflammation, atherosclerosis, psoriasis, atopic dermatitis, systemic lupus erythematosus (SLE) and cancer. More specifically, the present invention relates to monoclonal autoantibodies which have been isolated from B cells derived from subjects affected with an impaired central and/or peripheral tolerance or loss of self-tolerance typically due to a mutation in a gene involved in immune regulation.

In a further aspect, the present invention relates to a method for the assessment of binding of a ligand of interest to a ligand-binding domain and the identification of compounds present in a test sample, in particular antibodies that are capable of interfering with said ligand binding. More specifically, the present invention relates to a novel cellular assay for the assessment of binding of a ligand of interest to cells expressing the relevant receptor(s) for the ligand and the use of this assay for the assessment whether a candidate compound, e.g. antibody prevents or supports binding of the ligand to its cognate receptor(s) on the cells and thus has antagonistic or agonistic activity. In accordance with the most preferred embodiment of the assay described further below and illustrated in Examples and Figures, the assay of the present invention may also be referred to herein as Luminescent Cell Binding Assay (LCBA).

### BACKGROUND OF THE INVENTION

Interleukin-20 (IL-20, also known as interleukin-10D or ZCYTO10) is a pleiotropic cytokine with cell growth affecting, inflammatory and immunoregulatory functions (Blumberg et al. Cell 104 (2001), 9-19). The human IL-20 gene (NCBI Reference Sequence: NM_018724) is located on chromosome 1q32.1 and encodes a protein comprising 176 amino acids (aa) (UniProtKB/Swiss Prot identifier: Q9NYY1). IL-20 belongs to the IL-10 family which consists of nine members: IL-10, IL-19, IL-20, IL-22, IL-24, IL-26, and the more distantly related IL-28A, IL-28B, and IL-29 (Ouyang et al. Annu. Rev. Immunol. 29 (2011), 71-109). IL-10 family cytokines are essential for maintaining the integrity and homeostasis of tissue epithelial layers. Members of this family can promote innate immune responses from tissue epithelia to limit the damage caused by viral and bacterial infections. Depending on the context, IL-20 has potent inflammatory (Hsieh et al. Genes Immun. 7 (2006) 2434-242), angiogenic (Chen et al. Arterioscler. Thromb. Vasc. Biol. 26 (2006), 2090-2095) and chemoattractive characteristics (Hsu et al. Arthritis Rheum. 54 (2006), 2722-2733). However, an immunosuppressive role for IL-20 together with IL-19 and IL-24 during infection with methicillin-resistant Staphylococcus aureus has also been identified (Myles et al. Nature Immunol. 14 (2013), 804-811). IL-20 is preferentially expressed in five major cell types: epithelial cells, myoepithelial cells, endothelial cells, monocytes and skeletal muscle cells (Hsing et al. Cytokine 35 (2006), 44-52).

It is regulated by hypoxia and inflammatory stimuli such as IL-10 and lipopolysaccharide (LPS) (Otkjaer et al. J. Invest. Dermatol. 127 (2007), 1326-1336; Chen and Chang J. Immunol. 182 (2009), 5003-12). IL-20 induces signal transducer and activator of transcription (STAT) 1 and 3 activation either through IL-20 receptor (R) complex IL-20RA/IL-20RB (Type I) or through IL-20 receptor complex IL-22RA/IL-20RB (Type II) (Parish-Novak et al. J. Biol. Chem. 277 (2002), 47517-47523; Logsdon et al. Proc. Natl. Acad. Sci. USA 109 (2012), 12704-9). Both receptor complexes are expressed in skin and are dramatically upregulated in psoriatic skin and atopic dermatitis (Blumberg et al. Cell 104 (2001), 9-19; Toyhama et al. Eur. J. Immunol. 39 (2009), 2779-88). Overexpression of IL-20 in transgenic mice causes neonatal lethality with skin abnormalities including aberrant epidermal differentiation (Blumberg et al. Cell 104 (2001), 9-19; Liu et al. Blood 102 (2003), 3206-3209). IL-20 targets keratinocytes, endothelial and synovial cells and is associated with psoriasis (Wei et al. Clin. Immunol. 117 (2005), 65-72; Sa et al. J. Immunol. 178 (2007), 2229-2240).

Furthermore, IL-20 and its receptors have been observed to be overexpressed in rheumatoid arthritis (RA) synovial tissue biopsies and ankylosing spondylitis (AS) (Kragstrup et al. Cytokine 41 (2008), 16-23), wherein the expression levels of IL-20 and its receptors correlated positively with the severity of inflammation (Hsu et al. Arthritis Rheum. 54 (2006), 2722-2733). IL-20 induced RA synovial fibroblasts (RASF) to secrete monocyte chemoattractant protein (MCP)-1, IL-6 and IL-8, and it promoted neutrophil chemotaxis, RASF migration, and endothelial cell proliferation. A collagen-induced arthritis (CIA) rat model demonstrated that IL-20 was involved in the pathogenesis of arthritis, because soluble IL-20RA significantly reduced arthritis in rats with CIA (Hsu et al. Arthritis Rheum. 54 (2006), 2722-2733).

Besides its promoting role during the progression of rheumatoid arthritis (RA) and psoriasis, IL-20 was found to be functionally associated with several other disorders such as atherosclerosis (Chen et al. Arterioscler. Thromb. Vasc. Biol. 26 (2006), 2090-2095), acute renal failure (Li et al. Genes Immun. 9 (2008), 395-404), ulcerative colitis (Fonseca-Camarillo et al. J. Clin. Immunol. 33 (2013), 640-8), ischemic stroke (Chen and Chang J. Immunol. 182 (2009), 5003-12) as well as osteopenia and osteoporosis (Hsu et al. J. Exp. Med. 208 (2011), 1849-1861). Furthermore, IL-20 plays a role in the development of hematopoietic cells (Liu et al. Blood 102 (2003), 3206-3209).

Dysregulated expression of IL-20 genes has been observed in non-small cell lung (NSCL) cancer (Baird et al. Eur. J. Cancer 47 (2011), 1908-18) and in muscle invasive bladder cancer (Lee et al. PLoS One 7 (2012), e40267). IL-20 promotes migration of bladder cancer cells through extracellular signal-regulated kinase (ERK)-mediated MMP-9 protein expression leading to nuclear factor (NF-κB) activation by inducing the up-regulation of p21(WAF1) protein expression (Lee et al. J. Biol. Chem. 288 (2013), 5539-52). Expression of IL-20 and its receptor subunits was higher in clinical oral tumor tissue as well as breast cancer tissue than in non-tumorous tissue (Hsu et al. Mol. Cancer Res. 10 (2012) 1403-9, Hsu et al. J. Immunol. 188 (2012) 1981-91). In-vitro, IL-20 promoted tumor necrosis factor (TNF)-α, IL-1β, MCP-1, CCR4, and CXCR4 and increased proliferation, migration, reactive oxygen species (ROS) production, and colony formation of oral cancer cells via activated STAT3 and AKT/JNK/ERK signals. In-vivo, neutralizing IL-20 using the mouse anti-human IL-20 monoclonal antibody 7E reduced tumor growth and inflammation in oral cancer cells (Hsu et al. Mol. Cancer Res. 10 (2012) 1403-9).

In conclusion, IL-20 can promote aberrant cell growth, migration, and acts at local sites of inflammation in very different disease settings. Accordingly, IL-20 represents a not yet fully understood but important new therapeutic target and there is requirement for IL-20 specific binding molecules which are capable of neutralizing the biological activity and function of IL-20, which are useful in the treatment and diagnosis of disorders or conditions associated with detrimental IL-20 activity, and which are tolerable in humans.

The solution to this problem is provided by the embodiments of the present invention as characterized in the claims and disclosed in the description and illustrated in the Examples and Figures further below.

### SUMMARY OF THE INVENTION

The present invention relates to IL-20 specific human-derived monoclonal antibodies as well as fragments, derivatives and synthetic or biotechnological variants thereof that recognize IL-20. In particular, human monoclonal anti-IL-20 antibodies are provided with a selective binding profile towards IL-20 and displaying binding and neutralizing activity as shown in the appended Examples and Figures. Due to their neutralization properties, the antibodies of the present invention have therapeutic, prognostic and diagnostic utility, which make them in particular valuable for applications in relationship with diverse autoimmune and inflammatory disorders and conditions associated with/involving IL-20 activity in initiation and/or maintenance of undesired immune responses, such as various forms of arthritis (e.g., rheumatoid arthritis (RA) or spondyloarthritis), inflammatory bowel disease (IBD), Crohn's disease, psoriasis, vascular inflammation and atherosclerosis, atopic dermatitis and cancer; see also the background section above for these and further possible anti-IL-20 therapeutic and diagnostic indications.

The antibodies of the present invention have been isolated from a human affected with Autoimmune polyendocrinopathy syndrome type 1 (APS1) due to a mutation in the AIRE (Autoimmune Regulator) gene (Peterson et al., Nat. Rev. Immunol. 8 (2008), 948-957) and international application WO2013/098419 for review.

In this context, within experiments performed in accordance with the present invention first attempts to identify, isolate and characterize, in particular selecting the most potent neutralizing candidate antibodies appeared to be cumbersome. For example, hitherto as well as in initial experiments performed in accordance with the present invention the luciferase immunoprecipitation systems (LIPS) assay has been used to detect IgG antibodies against the desired antigen IL-20, which was reported to have the potential of monitoring antibodies to possible conformational epitopes, missed by ELISA, for assessing neutralizing activity; see Burbelo et al., Expert Rev. Vaccines 9 (2010), 567-578, author manuscript; available in PMC 2012, August 13. However, since in the experiments performed in accordance with the present invention the LIPS assay did not distinguish between neutralizing and non-neutralizing antibodies, the identification and selection of antibodies according to this method may not result in the provision of the actual most potent antibody in terms of its neutralizing activities.

Therefore, as illustrated in FIG. 7 and described in Example 5, in accordance with present invention a novel assay for the assessment of the binding of a ligand of interest to cells induced to express the relevant receptor(s) has been developed for use in determining whether candidate antibodies directed against the ligand prevent its ability to bind to the respective receptor on the cells. By way of example, cellular binding of the ligand is measured by cell-associated reporter, i.e. luciferase activity, as the ligand of interest is used as a reporter, i.e. luciferase fusion protein. This assay permits the assessment whether antibodies directed against the ligand prevent its ability to bind to the respective receptor on the cells by observing a change in reporter activity, i.e. reduced light emission by the luciferase. Hence, in accordance with the present invention a novel Luminescent Cell Binding Assay (LCBA) has been established. Thanks to this novel assay, it was possible to identify potent IL-20 neutralizing antibodies.

Thus, the subject antibodies illustrated in the Examples could be isolated and identified thanks to (i) a patient (designated APS1-9) being a high producer of anti-IL-20 antibodies and (ii) the use of a novel cellular ligand binding reporter assay.

In detail, the present invention is directed to:
[1] A human-derived monoclonal anti-human interleukin-20 (IL-20) antibody or an IL-20 binding fragment, synthetic or biotechnological derivative thereof, comprising in its variable region:
   (a) at least one complementarity determining region (CDR) of the V_{H} and/or V_{L} variable region amino acid sequences depicted in
      (i) FIG. 1 (V_{H}) (SEQ ID NOs: 2, 10, 18 and 26); and
      (ii) FIG. 1 (V_{L}) (SEQ ID NOs: 4, 12, 20 and 28);
   (b) an amino acid sequence of the V_{H} and/or V_{L} region as depicted in FIG. 1;
   (c) at least one CDR consisting of an amino acid sequence resulted from a partial alteration of any one of the amino acid sequences of (a); or
   (d) a heavy chain and/or light variable region comprising an amino acid sequence resulted from a partial alteration of the amino acid sequence of (b); preferably wherein the antibody is capable of reducing or neutralizing a biological activity of IL-20.
[2] The antibody or IL-20 binding fragment of [1], (1) which recognizes an IL-20 derived peptide consisting of the amino acid sequence 101-PDHYTLRKISSLANSFLT-118 (SEQ ID NO: 69), 102-DHYTLRKISSLANSF-116 (SEQ ID NO: 70) and/or 101-PDHYTLRKISSLANSFL-117 (SEQ ID No: 72), wherein only P101, 1109, S110 and/or L117 may be substituted by another amino acid, preferably alanine, and (2) which does not or does not substantially recognize a peptide consisting of the amino acid sequence 97-NYQTPDHYTLRKISSLAN-114 (SEQ ID NO: 71).
[3] The antibody or IL-20 binding fragment of [1] or [2], which is capable of binding and/or neutralizing murine IL-20.
[4] The antibody or IL-20 binding fragment of [2] or [3], wherein the biological activity is
   (a) human IL-20 signaling in a cell based STAT (signal transducers and activators of transcription) activation assay;
   (a) inhibition of IL-20 cytokine cell-surface receptor binding;
   (c) human IL-20 mediated activation of human IL20R1/IL20R2 receptor complexes; and/or
   (d) a pro-inflammatory activity of human IL-20.
[5] The antibody or IL-20 binding fragment of any one claims 1 to 4, wherein at least the CDRs are at least 70%, preferably 80% and more preferably at least 90% identical to the corresponding CDR indicated in FIG. 1 and/or the amino acid sequence of the framework region of the VH and/or VL variable region is at least 70%, preferably 80% and more preferably at least 90% identical to the corresponding framework region indicated in FIG. 1.
[6] The antibody or IL-20 binding fragment of any one of [1] to [5], which is Ig1 or IgG4.
[7] The antibody or IL-20 binding fragment thereof of any one of [1] to [6] comprising a C_{H} and/or C_{L} constant region comprising an amino acid sequence selected from the C_{H} and C_{L} amino acid sequences set forth in Table 1 (SEQ ID NOs.: 6, 14, 22 and 30) or an amino acid sequence with at least 60% identity.
[8] A human antibody or antigen-binding molecule thereof which competes with an antibody of any one of [1] to [7] for binding to human IL-20, for example to be determined in accordance with Example 2.
[9] The antibody of claim of any one of [1] to [8], which is selected from the group consisting of a single chain Fv fragment (scFv), an F(ab′) fragment, an F(ab) fragment, and an F(ab′)₂ fragment.
[10] A polynucleotide encoding at least the variable region of one immunoglobulin chain of the antibody or antigen-binding fragment of any one of [1] to [9], preferably wherein the polynucleotide is a cDNA encoding the variable region and at least part of the constant domain.
[11] A vector comprising the polynucleotide of [10].
[12] A host cell comprising a polynucleotide of [10] or a vector of [11].
[13] A method for preparing an anti-IL-20 antibody or immunoglobulin chain(s) thereof, said method comprising
   (a) culturing the cell of [12]; and
   (b) isolating said antibody or immunoglobulin chain(s) thereof from the culture, preferably wherein the anti-IL-20 antibody is an anti-human IL-20 antibody or an IL-20 binding fragment, synthetic or biotechnological derivative thereof as defined above and described in the following embodiments in the description, respectively.
[14] An antibody or immunoglobulin chain(s) thereof encoded by a polynucleotide of [11] or obtainable by the method [13].
[15] An immunoconjugate comprising the anti-IL-20 antibody or IL-20 binding fragment of any one of [1] to [9] or [14] or obtained by culturing the cell of [12], which preferably comprises a radionuclide, enzyme, substrate, cofactor, fluorescent marker, chemiluminescent marker, peptide tag, heavy metal, magnetic particle, drug, or a toxin.
[16] A composition comprising the anti-IL-20 antibody or IL-20 binding fragment of any one of [1] to [9] or [14], the immunoconjugate of [15], the polynucleotide of [10], the vector of [11], the cell [12], preferably wherein the composition is
   (a) a pharmaceutical composition and further comprises a pharmaceutically acceptable carrier and optionally further comprising an additional agent useful for treating an inflammatory disease; or
   (b) a diagnostic composition or kit and further comprises reagents conventionally used in immuno- or nucleic acid based diagnostic methods.
[17] An anti-IL-20 antibody or IL-20 binding fragment of any one of [1] to [9] or [14], the immunoconjugate of [15], or the composition of [16] for use in a method of:
   (a) treating or preventing the progression of an immune mediated or autoimmune disease or condition;
   (b) amelioration of symptoms associated with an immune mediated or autoimmune disease or condition; and/or
   (c) diagnosing or screening a subject for the presence or for determining a subject's risk for developing an immune mediated or autoimmune disease or condition associated with the expression and activity of IL-20 in a patient,
   preferably wherein said disease is selected from the group consisting of inflammatory bowel disease (IBD; including Crohn's disease, ulcerative colitis and celiac disease), ankylosing spondylitis and other forms of spondyloarthritis, psoriasis, psoriatic arthritis, rheumatoid arthritis, juvenile rheumatoid arthritis, osteoporosis, Sjörgen's syndrome, multiple sclerosis (MS), systemic lupus erythematosus (SLE), lupus nephritis, or a combination thereof, cancer including squamous cell carcinoma of the skin, tongue, esophagus or lung, and vascular inflammation.
[18] A method of preparing a pharmaceutical composition for use in the treatment of a disorder associated with the expression and activity of IL-20, the method comprising:
   (a) culturing the cell of [12];
   (b) purifying the antibody, biotechnological derivative or immunoglobulin chain(s) thereof from the culture to pharmaceutical grade; and
   (c) admixing the antibody or biotechnological derivative thereof with a pharmaceutically acceptable carrier.
[19] A method for diagnosing an immune mediated or autoimmune disease or condition in a subject associated with the expression of IL-20 comprising contacting a biological sample of the subject cell with an anti-IL-20 antibody or IL-20 binding fragment of any of [1] to [9], [14] or [15], and detecting the presence of IL-20.
[20] A method of detecting or determining IL-20 in an isolated biological sample comprising admixing the sample with an antibody according to any one of [1] to [9], [14] or [15], allowing the antibody to form a complex with IL-20 present in the mixture, and detecting said complex present in the mixture.

The present invention also makes use of and relates to embodiments of IL-20 antagonists, e.g. anti-IL-20 antibodies described in the prior art, which are adapted in accordance with the present invention by using the human-derived monoclonal anti-human IL-20 antibody or an IL-20 binding fragment, synthetic or biotechnological variant thereof disclosed herein. For example, antagonizing IL-20 activity using inter alia anti-IL-20 monoclonal antibodies has been described as a promising approach for treatment of various inflammatory conditions; see, e.g., international applications WO99/27103, WO01/46261, WO03/051384, WO2004/085475, and WO2006/086396. Rat or murine monoclonal antibodies binding human IL-20 have also been described; see, e.g. international applications WO2005/052000, WO2007/081465 and WO2010/000721. However, no human-derived antibodies suitable for patient treatment have so far been provided. The present invention addresses these and other needs in the art.

Furthermore, as illustrated in the Figures and described in the Examples, the present invention also provides a novel assay for the assessment of the binding of a ligand of interest to its cognate receptor(s) for use in determining whether a test substance, e.g. candidate compounds such as antibodies directed against the ligand prevent its ability to bind to the respective receptor(s) as well as for use as a competition and cross-competition assay for candidate anti-ligand antibodies.

In this novel assay, the relevant receptor(s) or antibodies are expressed in cells, wherein preferably at least the ligand-binding domain is exposed on the cell surface, and the ligand is provided as fusion molecule, i.e. being linked to or labeled with a reporter. Evidently, this novel assay does not only permit the assessment whether a candidate compound, e.g. antibody prevents binding of the ligand to its cognate receptor(s) on the cells and thus has antagonistic activity but also whether a candidate compound may enhance ligand binding and thus reporter activity, for example by stabilizing ligand-receptor binding, thereby identifying an agonist of ligand binding. In this context, the person skilled in the art will appreciate that the novel assay of the present invention is not only useful for the screening of anti-ligand binding molecules but also substances binding the cognate binding domain, i.e. receptor of the ligand and interfering with ligand binding. Therefore, the embodiments described herein for testing anti-ligand antibodies and the Examples could be equally applied to anti-ligand-binding domains, in particular receptors, preferably with an extracellular ligand-binding portion.

By way of example, cellular binding of the ligand is measured by cell-associated reporter, i.e. luciferase activity, as the ligand of interest is used as a reporter, i.e. luciferase fusion protein. This assay permits the assessment whether antibodies directed against the ligand prevent or enhance its ability to bind to the respective receptor on the cells by observing a change in reporter activity, i.e. reduced or increased light emission by the luciferase.

Hitherto, Luker et al. (Biotechniques 47 (2009), 625-632) described a luminescence-based assay for chemokine-chemokine receptor binding and suggested to use the bioluminescent reporter for probing chemokine receptors and the identification of small molecule modulators of chemokine-chemokine receptor binding. This assay has been further developed to an in vivo ligand receptor complementation assay in co-cultured live cells and animals; see Luker et al., Nature Medicine 18 (2012), 172-177.

However, a robust bioluminescent cellular in vitro assay as illustrated in the appended Examples and its use for the identification, isolation and/or validation of target protein neutralizing antibodies, in particular antagonists of interferons or interleukins has not been described or suggested. Accordingly, in this aspect the present invention relates to a novel cellular assay for the assessment of binding of a ligand of interest to cells expressing the relevant receptor(s) for the ligand. In its broadest aspect, the invention is directed to:
[21] A method for the assessment of binding of a ligand of interest to a ligand-binding domain comprising:
   (a) contacting a target cell (1) expressing the ligand-binding domain optionally comprising an intracellular functional region (2) with a sample comprising the ligand of interest (3) labeled with a reporter (4) which is preferably a detectable enzyme; and
   (b) determining reporter activity (5), preferably chemiluminescence;
   wherein an increase of reporter activity compared to a control indicates binding of the ligand of interest to the ligand-binding domain; and
   wherein the ligand-binding domain is derived from a receptor or an anti-ligand antibody, preferably wherein the ligand is an interferon or an interleukin.

The term “ligand” as used herein is given in the definition as provided in the Oxford Dictionary of Biochemistry and Molecular Biology, Oxford University Press, 1997, revised 2000 and reprinted 2003, ISBN 0 19 850673 2. A ligand includes any small molecule, nucleic acid, protein or antigen of interest, for example but without limitation, CD marker ligands such as CD48, CD40L, CD40, CD 122, cytokines and chemokines such as IL-2, IL-12, IL-13, IL-15, IL-17, IL-6, TNF-a, ILβ, IL-10, IP-10, growth factors such as ITAC, MIG, EGF, VEGF, co-stimulatory ligands such as ICOS-L, OX-40-25 L, CD137-L, components of signaling pathways, and indeed, any antigen of interest. A ligand also includes an antibody. In the methods of the invention, the ligand is most preferably a soluble ligand.

A reporter is a detectable moiety and may be of any kind that can be traced, either directly or indirectly. Many such moieties are well known in the art and include standard labels such as those based on radioactivity, fluorescence, chemiluminescence, plasmonresonance of colloidal metals, or enzymatic-colorimetric detection that are used routinely in biochemistry, cell biology, and medical diagnostic applications.

A “ligand-binding domain” may be any molecule, which is capable of binding a ligand such as defined hereinabove. Typically, the ligand-binding domain may be derived from a protein or polypeptide such as a receptor molecule. However, in principle also non-proteinaceous binding molecules may provide a ligand-binding domain in accordance with the present invention, for example synthetic binding molecules; see, e.g., Zeng et al., ACS Nano 4 (2010), 199-204 describing synthetic polymer nanoparticles with antibody-like affinity for a hydrophilic peptide. Nevertheless, in a preferred embodiment, the ligand-binding domain will be derived from a receptor or an antibody, typically an anti-ligand antibody. A ligand-binding domain may be any polypeptide or part thereof, which is capable of binding a ligand such as defined hereinabove. Receptors are ligand-activated binding proteins that regulate signal transduction pathways including gene expression. The mechanism of action for these receptors involves as a first step activation through binding of the ligand and as a second step signal transduced by the receptor triggering or inhibiting downstream signaling cascades which culminate in the regulation of gene expression. The domain responsible for ligand binding is the ligand-binding domain (LBD). This domain participates in several activities including ligand binding and homo- and/or heterodimerization. The conformational changes that accompany the transition between the liganded and unliganded forms of receptors dramatically affect their affinity for other proteins (Edwards, J. Mammary. Gland Biol. Neoplasia 5 (2000) 307-24; Thomas-Chollier et al., Proc. Natl. Acad. Sci. USA 110 (2013) 17826-31).

However, for the purposes of the method and assay of the present invention, the presence of the ligand-binding domain present extracellularly on the cell membrane is sufficient since in contrast to most bioassays in the prior art the method and assay of the present invention is not dependent on intracellular signaling for recording reporter activity. Nevertheless, the ligand-binding domain may be fused or bound to one or more functional regions, for example a transmembrane region.

A transmembrane region generally serves to anchor a receptor to the cell membrane (thus the extracellular ligand-binding domain is membrane-bound) and includes any protein capable of doing so (or nucleic acid encoding such a protein). Such a region can be derived from a wide variety of sources such as all or part of the alpha, beta, or zeta chain of the T cell receptor (TCR), CD28, CD4, CDS, CD8, CD3a, CD16, CD22, CD23, CD45, CD80, CD86, CD64, CD9, CD37, CD122, CD137 or CD154, a cytokine receptor such as an interleukin receptor, TNF-R, a tyrosine kinase receptor or interferon receptor, or a colony stimulating factor receptor. Alternatively, the transmembrane region may be synthetic. Suitable synthetic transmembrane regions will comprise predominantly hydrophobic amino acids such as leucine and valine.

The term “target cell” as used in the context with the present invention includes any cell, which is capable of expressing one or more ligand-binding domain(s) of interest. In particular, mammalian cells and cell lines may be used which either endogenously express one or more receptors capable of binding to the ligand of interest or which can be transfected or transformed such as to express one or more said receptors. However, meanwhile artificial cells and cell membranes have been developed in which receptors can be inserted for studying ligand binding; see, e.g., for review Hammer and Kamat, FEBS Letters 586 (2012) 2882-2890 and May et al., Angewandte Chemie, International Edition 52 (2013), 749 DOI: 10.1002/anie.201204645.

The method and assay of the present invention may be performed by contacting the target cell (1) expressing the ligand-binding domain optionally comprising an intracellular functional region (2) with a sample comprising the ligand of interest (3) labeled with a reporter (4). If a cognate ligand is present, a complex is formed among the two binding reagents and the ligand is bound to the target cell. Unbound ligand of interest is separated from the reaction mixture, for example by washing, and the presence of the ligand bound to the target cell is detected via the reporter moiety (6), for example due to its enzymatic activity towards a substrate (5). The presence (or magnitude) of the reporter activity in the target cell-ligand complex is indicative of the presence (or concentration) of the ligand. The method and assay of the present invention can be performed in any appropriate container, vial, culture dish, multiple well plates, liquid capillary systems, and the like. Alternatively, the method and assay of the present invention may be performed such that the cell having the ligand-reporter bound to the ligand-binding domain is selectively detected, for example by Fluorescence-activated cell sorting (FACS) when using a fluorescent moiety as the reporter.

As illustrated in the Examples, the method and assay of the present invention are particularly suitable for assaying receptor ligand-binding and specifically the interaction of a cytokine, most preferably an interferon or an interleukin, with its cognate receptor(s).

Cytokines are small glycoprotein messengers, which where variously identified in the absence of a unified classification system by numeric order of discovery, a given functional activity, a kinetic or functional role in inflammatory responses, a primary cell of origin, or structural homologues shared with related molecules; see, e.g., McInnes in Kelley's Textbook of Rheumatology, Elsevier Health Sciences 9th Edition Vol. 1 by Firestein et al. (2012), 3rd part, chapter 23, 367-377. The cytokines and receptors used in accordance with the present invention are described but not limited to the cytokines and receptors described in McInnes (2012) 3rd part, chapter 23, 367-377) depicted in Tables 23-1 to 23-8, the disclosure content of which is incorporated herein by reference, and in the cytokine listing disclosed in international applications WO2013/098419 and in applicant's co-pending international application WO2015/001047. Preferably, the cytokine is a human cytokine. In a preferred embodiment, the cytokine is selected from the group consisting of leukotrienes, lymphokines, interleukins, interferons, chemokines and members of the TNF family. However, in principle the ligand may be virtually any soluble molecule including hormones and growth factors but also small molecules such as carbohydrates capable of interacting with a cognate binding domain such as a receptor or antibody.

Hence, experiments performed in accordance with the present invention surprisingly revealed that the LBCA assay is particularly suited for testing candidate anti-ligand antibody, wherein by way of expression of a transmembrane version thereof the anti-ligand antibody serves as the anti-ligand binding domain; see Example 9 and FIG. 17 describing the expression of anti-IFN-alpha and IFN-w antibody 26B9 in 293T MSR cells and its efficient binding to luciferase tagged IFN-w. Thus, the method and assay of the present invention are also particularly suitable for assaying antibody-ligand binding. Accordingly, in a further preferred embodiment, the present invention relates to
[22] The method of [21], wherein the ligand-binding domain is an antibody or a ligand/antigen-binding fragment or derivative thereof.

Also in this embodiment, the ligand/antigen may preferably be a cytokine while however apparently any other ligand/antigen interaction may be assayed as well.

As discussed in the background section and illustrated in the Examples, the initial screening of antibodies with a desired specificity makes use of biochemical assays such as ELISA and LIPS assay which are usually sufficient for determining specific binding for example of an antibody to a ligand. However, since those assays almost always are performed under non-cellular, i.e. non-physiological conditions, such biochemical assays may not reliably predict whether a given antibody may also be capable of binding to the ligand in-vivo and interfere with its biological activity, for example inhibiting the binding of the ligand to a receptor thereby triggering a signal transduction pathway.

In contrast, as demonstrated in Example 5, the cell-based assay of the present invention, wherein the ligand binding is assessed in a cellular context is reliable in determining the neutralizing activity of anti-ligand molecules as confirmed by the functional STAT3 assay described in Example 5. Therefore, the cell-based ligand-binding assay of the present invention obviates the need for performing such functional assays, thereby reducing laboratory equipment, time and costs. Accordingly, in one embodiment, the present invention relates to:
[23] The method of [21] or [22], wherein the ligand-binding domain and/or the ligand are biologically active.

Although in principle any cell, ligand and ligand-binding domain may be investigated, the assay of the present invention is particularly suitable for the assessment of mammalian and in particular human ligands and ligand-binding domains for which reason also the use of mammalian and specifically human target cells are preferred. Therefore, in a further preferred embodiment the present invention relates to:
24] The method of any one of [21] to [23], wherein the target cell, ligand and/or ligand-binding domain is of mammalian, preferably human origin. However, the target cell is not necessarily of mammalian origin, but could be any cell, including prokaryotes. The ligand-binding domain can also be part of non-living cell-like particles, including but not limited to virus-like particles (VLPs), synthetic cells or other cell-like particles.

An extracellular ligand-binding domain comprises any protein (or nucleic acid encoding such a protein) that is capable of binding a protein ligand. Most preferably, the ligand is a soluble ligand. Membrane-bound extracellular ligand-binding domains include or are derived from, e.g., surface membrane receptors, such as kinase receptors, G-protein coupled receptors (GPCRs), growth factor receptors, cytokine receptors such as interleukin receptors, e.g. IL-IR (Type I and II), IL-2R (α, β and γ subunits), IL-3R, IL-4R, IL-5R, IL-6R; gp130, IL-8R, IL-13Ral, IL-4Ra, IL-30R, IL-15R (α, β and γ subunits), IL-17R; TNF receptor(s) (TNF-RI and TNF-RII); receptors for IL-β, IL-2, IL-10, IL-15, G-CSF, CSF-1, M-CSF, GM-CSF, HGF, EGF, PDGF, IGF, FGF, TGF-β, IP-10, ITAC, MIG and VEGF; CD markers such as CD2, CD4, CDS, CD7, CD8, CD11a, CD11b, CD11c, CD11d, CD16, CD19, CD20, CD22, CD24, CD28, CD33, CD40, CD48, CD69, CD70, CD122 and CD244; and receptors for ICOS-L, OX-40-L, CD40L and CD137-L. Membrane-bound extracellular ligand-binding domains for use in the method and assay of the present invention can also include molecules as binding domains that are not normally found on or within the cellular membrane, for example, a cytosolic or soluble protein, for example but without limitation, a protein which is normally secreted. Thus, extracellular ligand-binding domains can also include SOST, and LDL-related proteins such as LRPS and LRP6, cytokines, and growth factors that, by virtue of a transmembrane region, see infra, are presented extracellularly. It will be understood by those skilled in the art that preferably a signal sequence targeting the extracellular ligand-binding domain to the cell surface is included in the DNA sequence of the vector encoding the ligand-binding domain and receptor, respectively. Such signal sequences are known in the art and include sequences that are naturally associated with the extracellular ligand-binding domain (signal sequences/leader sequences). A signal sequence will generally be processed and removed during trafficking of the extracellular ligand-binding domain to the cell surface.

In one embodiment, an extracellular ligand-binding domain can be chosen such that it interacts with one or more other extracellular ligand-binding domains to achieve multiply associated extracellular ligand-binding domains capable of recognizing (binding to) a ligand. Thus, in one embodiment, the target cell in the method and assay of the invention comprises more than one membrane-bound extracellular ligand-binding domain. More preferably, two or more different ligand-binding domains may be expressed in the target cell to achieve multiply associated extracellular ligand-binding domains capable of recognizing (binding to) a ligand.

As explained in the legend to FIG. 7A, the reporter may be any detectable moiety. A reporter moiety includes compounds capable of generating a measurable (detectable) signal, such as and without limitation, luciferase, secreted alkaline phosphatase (SEAP), green fluorescent protein or red fluorescent protein. Thus, reporter signal measurements include measurement of light emission, fluorescence or alkaline phosphatase production. For review of an appropriate reporter molecule; see, e.g. Lesner Biochem. Anal. Biochem. 4 (2012), 1-2; Kain and Ganguly Curr. Protoc. Mol. Biol. (2001) Chapter 9: Unit 9.6). Accordingly, in one embodiment, the present invention relates to:
[25] The method of any one of [21] to [24], wherein the reporter is a detectable enzyme, preferably selected from the group consisting of luciferase, SEAP, horseradish peroxidase, and alkaline phosphatase, preferably wherein the detectable reporter activity is light emission (luminescence, chemiluminescence).

Renilla luciferase (abbreviated herein as “Ruc”) is particularly preferred. Most preferably, as illustrated in the Examples, the reporter is Gaussia luciferase which is highly sensitive, naturally secreted, and can be detected in the conditioned medium of cells in culture as well as in the blood of animals ex-vivo, allowing real-time monitoring of cellular variables (Tannus et al. Mol. Ther. 11 (2005), 435-43; Wurdinger et al. Nat. Methods. 5 (2008); 171-3) Alternatively, the reporter can also be NanoLuc® Luciferase; see, e.g. Hall et al. ACS Chem. Biol. 7 (2012), 1848-1857. In another embodiment, the detectable moiety comprises multiple copies of a detectable enzyme. Such may be accomplished by, for example, the use of a cloning vector coding for multiple copies of the enzyme, which may be linked in tandem, or located on either side of the binding domain. Other detectable moieties include, for example, fluorescent proteins such as green fluorescent protein, and various other colored proteins sold by, e.g., Clontech in their Living Colors™ product line.

However, as mentioned luciferase and equivalent light emitting enzymes are particularly preferred. For example, assays with fluorescently labeled ligands suffer from background autofluorescence of cells, plasticware and buffers. In contrast, there is no background light emission in the LCBA assay of the present invention because luciferases are only found in a few very specialized animals and not in mammals. As a further advantage, the LCBA assay also works when the cellular receptor is unknown as long as there is a responsive cell line. Finally, one major advantage of the cell-binding assay of the present invention is the fact that it allows assessing the function of e.g. an antibody at a point in the signal transduction cascade that is maximally upstream. Therefore, the assay is not influenced by e.g. other signaling molecules present in the reaction mixture, which might trigger interfering downstream signaling events. Thus, very complex mixtures such as serum can be assayed for the presence of neutralizing antibodies.

In some assays described in the prior art, the reporter-antigen fusion protein is not secreted but expressed intracellularly which demands to lyse the cells to harvest the fusion protein. Since this is rather time-consuming, in a preferred embodiment of the assay of the present invention the labeled ligand is a recombinant fusion that is preferably secreted. Accordingly, in one embodiment, the present invention relates to:
[26] The method of any one of [21] to [25], wherein the labeled ligand is a recombinant fusion protein, preferably expressed and secreted by a host cell.

The target cell used in the method of the present invention may be any cell that naturally or genetically engineered expresses one or more ligand-binding domains. For example, genetically engineered cell lines, in particular mammalian cell lines may be established which either constitutively or upon induction express the ligand-binding domain of interest. Suitable induction systems such as the TET-repressor system are well known to persons skilled in the art. Alternatively, in particular with a view of investigating several kinds of ligand-binding interactions, the target cell may be genetically engineered on time to express the ligand-binding domain, for example by transfection of an appropriate expression vector encoding the ligand-binding domain at the onset of performing the method of the present invention. Similarly, also a ligand-reporter fusion molecule may be prepared on demand, for example in case of a fusion ligand-reporter protein by transiently expressing the fusion protein in a host cell; see also the appended Example 5.

Mammalian target and host cells may be transfected with an expression vector using any convenient technique such as electroporation, calcium phosphate transfection, DEAE-dextran mediated transfection, transvection, microinjection, cationic lipid mediated transfection, electroporation, transduction, scrape loading, ballistic introduction or infection (see e.g. Davis et al. Basic Methods in Molecular Biology, 1986 and Sambrook et al. Molecular Cloning: A Laboratory Manual, 2nd Ed. Cold Spring Harbour laboratory Press, Cold Spring Harbour, N Y, 1989). Suitable conditions for inducing vector expression within a host cell are well known in the art. Transfection may be transient or, alternatively, cells lines stably expressing a ligand-binding domain may be produced as known in the art. In particular, see Methods in Molecular Biology 7. Gene Transfer and Expression Protocols. Edited E. J. Murray 1991. On the other hand, also target cells may be used which endogenously express the ligand-binding domain of interest; see, e.g., HEK 293T MSR cells and COLO 205 colorectal carcinoma cells used in Example 5. For example, cells and cell lines, respectively, can be used which express growth factor or cytokine receptors such as interleukin-dependent cell lines. Furthermore, in view of the experiments described in Example 9 with respect to membrane bound antibody as a ligand-binding domain cells that express an antigen-binding domain may be used as a target cell including B- and T-cells which express membrane bound antibody and T-cell receptors, respectively.

Accordingly, in a further preferred embodiment, the present invention relates to:
[27] The method of any one of [21] to [26], wherein the target cell endogenously, transiently or stably expresses the ligand-binding domain and/or the method of [26], wherein the host cell transiently expresses the fusion protein.

In principle, the ligand-reporter fusion may be produced separately and via biochemical means, respectively, for example by in-vitro translation or chemical linking of the ligand of interest with the reporter moiety. Alternatively, it is also possible to link the reporter moiety to the ligand of interest indirectly via physical interaction, for example high affinity systems such as streptavidin/biotin or direct labeling with a metal such as preferably colloidal gold. In principle, this would also be possible for a fusion protein such as a fusion of a cytokine and luciferase which can be provided in an isolated and solid form.

However, a disadvantage of the use of chemically produced and/or isolated fusion proteins may be that because of their preparation and/or during for example the isolation process, drying and/or lyophilization the ligand component does no longer have the native conformation necessary to reflect the binding of the ligand to its binding domain in vivo and that the native structure is also not rebuilt in a liquid sample. This is particular true for cytokines and hormones having cysteine residues and disulfide bridges, respectively, such as G-CSF, which during their preparation tend to denature and can be refolded only under harsh conditions.

In contrast, as illustrated in the Examples, in accordance with the present invention the ligand-reporter fusion protein can be transiently expressed in the host cell and secreted in the culture medium. The culture medium and supernatant of the culture, respectively, containing the fusion protein which is supposed to comprise the ligand of interest in its native form can directly be used in the method of the present invention without any immediate purification steps; see Example 5. In this embodiment, it is most appropriate to use as control a culture medium and supernatant, respectively, obtained from a host cell that does not express and secrete the fusion protein or which expresses and secretes a fusion protein with a different ligand. Accordingly, in a particularly preferred embodiment, the present invention relates to:
[28] The method of [26] or [27], wherein the sample comprises a supernatant of a culture of the host cell, preferably wherein the control is a culture medium without the supernatant or a supernatant of a culture of the same kind of cell without expressing and secreting the fusion protein.

In principle, any kind of cell capable of expressing a ligand-binding domain and secreting the fusion protein, respectively, may be used in the method of the present invention with mammalian cells being preferred for use as the target and host cell. Mammalian host cells including Jurkat cells, HEK 293, CHO, NIH3T3, NSO, Cos-7, Hela, MCF-7, HL-60, EL4, A549, and K562 cells as well as appropriate expression vectors configured to contain and express a DNA encoding a ligand-binding domain, i.e. bioassay receptor with an extracellular ligand-binding domain capable of binding to a ligand and a transmembrane region are described in, e.g., international application WO2007/060406 the disclosure content of which incorporated herein by reference. However, in contrast to the bioassay disclosed in international application WO2007/06040 the receptor for use in accordance with the method of the present invention does not need to contain a reporter region and one or more intracellular signaling regions capable of transmitting a signal. Rather, the expression of the extracellular ligand-binding domain and optionally of a transmembrane region or another membrane anchoring polypeptide, either or both of which may be homologous or heterologous to the extracellular ligand-binding domain are sufficient for the purposes of the method of the present invention.

Preferably, the host and target cell, respectively, is a cell as used in the appended Examples, i.e. human embryonic kidney (HEK) 293T MSR cell or a COLO 205 colorectal carcinoma cell. Accordingly, in another preferred embodiment, the present invention relates to:
[29] The method of any one of [21] to [28], wherein the target cell and/or host cell is a HEK 293T MSR cell or a COLO 205 colorectal carcinoma cell. Preferably, the target cell is a HEK 293T MSR cell transiently expressing Type I and Type II IL-20 receptors and the sample comprises a supernatant of HEK 293T cells expressing IL-20-luciferase fusion protein; or the target cell is a HEK 293T MSR cell transiently expressing a transmembrane anti-interferon omega (IFNW) antibody and the sample comprises a supernatant of HEK 293T cells expressing IFNW-luciferase fusion protein.

As shown in the Examples, the method of the present invention gives similar results irrespective whether the target cell is lysed or not lysed prior to step (b). This is advantageous since the target cell does only have to be viable and the cell membrane in a non-denatured state, respectively, in step (a) for contacting the target cell with the ligand of interest linked with the reporter. After this step, the method of the present invention can be performed in similar manner as biochemical assays such as ELISA or the LIPS assay under relative robust conditions. Accordingly, alternatively, the present invention relates to:
[30] The method of any one of [21] to [29], wherein the target cell is lysed or not lysed prior to step (b).

As illustrated in FIG. 7 and discussed herein before, the method of the present invention in view of its reflection of ligand-binding in-vivo and its sensitivity is also particularly suited for determining whether a test substance is capable of interfering with ligand binding, i.e. positively or negatively and thus acts as an agonist or antagonist of binding of a ligand of interest to its cognate ligand-binding domain(s). Thus, in a further important aspect the present invention relates to:
[31] An assay for determining whether a test sample comprises an agonist or antagonist of binding of a ligand of interest to its cognate ligand-binding domain comprising the steps of the method of any one of [21] to [30], wherein prior or during step (a) the ligand and/or the target cell is subjected to the test sample (7) and in step (b), compared to the reporter activity in the absence of the test sample or in the presence of a control
   (i) an increase of reporter activity is indicative for an agonist; and
   (ii) a decrease of reporter activity is indicative for an antagonist.

The assay of the present invention is illustrated in FIG. 7 and further described in Example 5. Briefly, in the above-described method of the present invention it is assessed whether a test substance or for example an antibody directed against the ligand of interest prevents its ability to bind to the respective ligand-binding domain(s) such as the cognate receptor(s) of the ligand on the target cells. Thus, the ligand of interest prior to step (a) may be contacted with the test sample and test substance, respectively, wherein upon binding of the test substance to the ligand of interest its binding to the ligand-binding domain is blocked. Accordingly, the reporter activity which usually will be observable upon binding of the ligand-reporter fusion molecule decreases or is absent all together thereby indicating the presence of an antagonist of ligand binding. Alternatively, the target cell may be contacted with the test sample and test substance, respectively, prior to step (a), wherein upon binding of the test substance to the ligand-binding domain its capability of binding a ligand of interest would be blocked, which in turn again leads to a decrease or even absence of reporter activity and thus indicates the presence of an antagonist. Furthermore, particularly for determining an agonist of ligand binding, the test sample and test substance, respectively, is contacted with the test cell and the ligand of interest during step (a), where upon for example binding of a test substance to a complex of the ligand of interest with its cognate ligand-binding domain(s) the binding of the ligand of interest to a binding-domain may be stabilized and as a result leads to quantitative higher binding of the ligand and thus an increase of reporter activity indicating the presence of an agonist. In summary, the person skilled in the art will well recognize the possibilities of using the assay of the present invention for determining an agonist or antagonist of binding of a ligand of interest to its cognate ligand-binding domain(s).

The term “agonist” and “antagonist”, respectively as used in the context with the present invention though being illustrated with antibodies and biotechnological derivatives thereof also includes other, non-antibody molecules that are capable of binding to the ligand of interest to its cognate ligand-binding domain(s) or a complex of both, thereby agonizing, e.g. supporting ligand-receptor binding or antagonizing, e.g. interfering with the interaction of the ligand with its binding domain(s). Thus, the compound in the test sample includes antibodies, small molecules (e.g. NCEs) and other drugs, proteins, polypeptides and peptides, peptidomimetics, lipids, carbohydrates and nucleic acids, ankyrins, receptors, major histocompatibility complex (MHC) molecules, chaperones such as heat shock proteins (HSPs) as well as cell-cell adhesion molecules such as members of the cadherin, intergrin, C-type lectin and immunoglobulin (Ig) superfamilies. Nevertheless, for the sake of clarity only and without restricting the scope of the present invention most of the following embodiments are discussed with respect to antibodies and antibody-like molecules which represent the preferred “agonist” and “antagonist” for the development of therapeutic and diagnostic agents. Most preferably, the compound of interest present in the test sample is an antibody.

In addition, as explained above with reference to Example 9, the assay of the present invention may also be applied as a competition and cross-competition assay, respectively, for example for investigating whether candidate anti-ligand antibodies bind to the same or distinct epitopes of a given ligand; see Example 9 and FIG. 18 illustrating a cross-competition assay in accordance with the present invention, where it is shown that two anti-interferon antibodies 8H1 and 26B9 bind to distinct epitopes of interferon-w. Accordingly, in one embodiment of the assay of the present invention, the target cell expresses a membrane bound antibody or ligand-binding domain thereof and the agonist and antagonist, respectively, of binding of a ligand of interest may be the same kind of anti-ligand antibody or like candidate anti-ligand antibodies. Thus, in this embodiment the assay of the present invention is used as a competition and cross-competition assay, respectively, wherein the ligand-binding domain and agonist/antagonist are derived from the same ligand-binding molecule.

In the Examples and Figures, the assay of the present invention is illustrated with respect to the identification and isolation of anti-ligand antibodies. However, as will be understood by the person skilled in the art, the assay of the present invention is also capable of identifying and isolating compounds which interact with the ligand-binding domain, for example the receptor of the ligand. For example, an antibody recognizing the ligand-binding domain of a receptor may prevent binding of the ligand to a receptor, thereby decreasing a reporter activity because of loss of the reporter tagged ligand from the sample to be analyzed and thus indicating the presence of an antagonistic antibody.

Unless indicated otherwise, the term “test sample” may be used herein interchangeably with the term “test substance” or “test compound” and usually indicates that a test substance typically is provided as a sample, for example present in a solvent and/or taken from a batch of test substances. Similarly, since the assay of the present invention can be used at any stage of for example drug development, the test sample can be, e.g., a reaction mixture from chemical synthesis, a sample of a natural source such as soil sample, water sample, preparation of microorganism, body fluid, and the like, samples taken from intermediate steps of food and drug production as well as samples from pre-clinical batches of drugs before pharmaceutical use. Thus, the “test sample” can be any kind of matter which contains a putative agonist or antagonist, either alone or in context with other constituents. As illustrated in the Examples, the test sample may be preferably a body fluid such as blood plasma containing an antibody or equivalent molecule that is capable of binding the ligand of interest, the ligand-binding domain and/or the complex of the same.

Accordingly, in a particular preferred embodiment, the present invention relates to:
[32] The assay of [31], wherein the test sample comprises (i) an anti-ligand antibody, ligand-binding fragment, synthetic or biotechnological derivative thereof or (ii) an anti-ligand receptor (LR) antibody, LR-binding fragment, synthetic or biotechnological derivative thereof, preferably wherein the antibody is a human-derived antibody; and optionally wherein the control is a non-ligand- or non-LR binding antibody.

As explained further below, the assay of the present invention is particularly suited for determining the presence of an antibody or equivalent binding molecule with desired specificity in a sample derived from subject, e.g., mammal which has been immunized with an antigen, e.g., the ligand of interest or otherwise may be expected to produce an anti-ligand antibody, for example because of an immune response in the mammal evoked by an internal or external stimulus such as a disorder or pathogenic infection, stress, poisoning, and the like. On the other hand, since it is known that certain level of exercise or positive stress activate the immune system and may protect against common diseases such as cold, flu, cancer, and the like, the subject may have performed a corresponding experience well before the sample is taken.

Accordingly, in a preferred embodiment, the present invention relates to:
[33] The assay of [31] or [32], wherein the sample is derived from a body fluid, preferably sera obtained from a subject suspected to contain an anti-ligand or anti-LR antibody, preferably wherein the subject suffers from an autoimmune or inflammatory disorder, pathogenic infection, cancer, a subject with remarkable disease course, i.e. stable disease, being in remission, or having overcome disease, as this may indicate the presence of a disease combatting antibody or the subject has performed an exercise or experienced positive stress.

As illustrated in Example 5 and shown FIGS. 7, 8 and 14, different ways to perform the method and assay of the invention exist. For example, in order to determine the specificity of ligand-binding and identify competitive inhibitors, respectively, the cellular ligand-binding reporter assay may be performed in the presence of a competitor, e.g., ligand or inhibitor, for example, the same ligand but without the reporter or a ligand which is different but closely related to the ligand of interest and which may either be unlabeled or labeled with a reporter which is different from the reporter linked to the ligand of interest; see also the embodiments described further below, in particular with respect to the Dual Luciferase assay. The (putative) competitive ligand may be added prior, concomitantly or after the test cell has been subjected to the reporter-tagged ligand of interest, for example, in order to get information on the affinity of the ligand of interest or the biological activity of the competitive ligand, for example, whether it is capable of displacing the ligand of interest after ligand binding. Likewise, alternatively or in addition the test sample with the test substance may be added before, concomitantly or after contacting the target cell with the ligand. Depending on whether the test substance is suspected to bind to the ligand of interest or to the ligand-binding domain present on the target cell or both, either the ligand of interest or the target cell may be pre-incubated with the test substance. In this context, molecules that do not bind the ligand and the ligand-binding domain, respectively, may be added preferably in excess prior, during or after incubation of the ligand of interest and the target cell with the test sample. Accordingly, in one embodiment, the present invention relates to:
[34] The assay of any one of [31] to [33], wherein the ligand or the target cell is pre-incubated with the test sample, or as a control with a non-ligand- or non-LR-binding molecule or excess concentrations of an unlabeled ligand and/or a ligand which is different and unlabeled or labeled with a different reporter. Preferably, the control ligand is closely related to the ligand of interest and/or the control non-ligand- or non-LR-binding molecule is closely related to the test substance.

In this context, the assay of the present invention also allows determining the capability of the test sample and test substance, respectively, to discriminate for example between binding to a similar but different cytokine and/or their binding to different sites of the ligand-binding domain, for example within one or more cytokine receptor(s). For doing so, in a preferred embodiment one or more different antigens of interest, for example cytokines are used and labeled with reporter moieties which give rise to different signals which preferably can be assessed sequentially or concomitantly, for example because of the emission of light of different wave lengths in case different luciferases or fluorescent proteins are used as the reporter. In this embodiment, for example the luciferase reporter assay system (The Dual-Luciferase® Reporter (DLR™) Assay System) offered by Promega Corporation, 2800 Woods Hollow Road, Madison, Wis. 53711 USA, can be adapted and used in accordance with the present invention, wherein one cytokine is tagged to firefly luciferase and a second cytokine to Renilla luciferase. In accordance with the assays of the present invention, the activities of firefly (Photinus pyralis) luciferase fused to a first ligand of interest and Renilla (Renilla reniformis or sea pansy) luciferase fused to a second ligand of interest are measured sequentially from a single sample. The firefly luciferase reporter is measured first by adding a luciferase substrate (e.g. Luciferase Assay Reagent II (LAR II)) to generate a luminescent signal lasting at least one minute. After quantifying the firefly luminescence, this reaction is quenched, and the Renilla luciferase reaction is initiated simultaneously by adding Stop & Glo® Reagent to the same.

Thus, in this embodiment it is possible to easily determine whether a given test substance, for example an anti-cytokine antibody shows a preferential binding to one cytokine over another or to a ligand-binding site of a receptor for one cytokine while, if present, leaving another ligand-binding domain unaffected in kind.

In principle, the candidate antibody or equivalent human cytokine-binding agent could be provided by any possible source including the classical hybridoma approach as well as combinatorial libraries for producing synthetic antibodies and antibody-like cytokine binding molecules. Thus, antibody engineering has become a well-developed discipline, encompassing discovery methods, production strategies, and modification techniques for natural and non-natural human antibody repertoires and their mining with non-combinatorial and combinatorial strategies. This also includes the production and selection of human antibodies (mAbs) from naïve, immune, transgenic and synthetic human antibody repertoires using methods based on hybridoma technology, clonal expansion of peripheral B cells, single-cell PCR, phage display, yeast display, mammalian cell display and in silico design; see for review of the characteristics of natural and non-natural human antibody repertoires and their mining with non-combinatorial and combinatorial strategies, e.g., Beerli and Rader in Mining human antibody repertoires, mAbs 2 (2010); 365-378 and Campbell et al., British Journal of Pharmacology 162 (2011), 1470-1484; the disclosure content of which is incorporated herein by reference.

However, as mentioned before, in a preferred embodiment of the method of the present invention, the candidate anti human cytokine antibody or cytokine binding fragment thereof is derived from a human antibody provided by a method comprising isolating a monoclonal antibody or antigen-binding fragment thereof, wherein a B cell expressing the monoclonal antibody is isolated from a sample obtained from a subject mammal. In the past decades several technologies have been developed to isolate monoclonal antibodies and to produce humanized or fully human antibodies; see, e.g., references cited in international application WO 2007/068758 also granted as European patent EP 1 974 020 B1, in particular in sections [0002] to [0027], the disclosure content of which is incorporated herein by reference. Typically, the isolation of antibodies, for example monoclonal antibodies from B cells relies on cloning and expression of the immunoglobulin genes. This can be done by using phage display libraries of scrambled V_{H} and V_{L} genes from B cells, or by isolation of paired V_{H} and V_{L} genes from single B cells using single cell PCR or from immortalized B cell clones.

In accordance with the present invention the use of candidate anti human cytokine antibodies which originated from B cells of patients suffering from an autoimmune and/or inflammatory disease turned out to be in particular valuable, i.e. in particular for providing human antibodies. Accordingly, in one embodiment, the present invention relates to:
[35] A method of preparing a human-derived monoclonal anti-ligand antibody or a ligand-binding fragment, synthetic or biotechnological derivative thereof, wherein a B cell expressing the monoclonal antibody is isolated from a sample obtained from a mammal, preferably wherein the mammal is affected with an impaired central and/or peripheral tolerance or loss of self-tolerance such as autoimmune polyendocrinopathy-candidiasis-ectodermal dystrophy (APECED), the method comprising
   (a) assaying the sample for the presence of an anti-ligand antibody;
   (b) purifying B cells, preferably memory B cells from a sample which has been identified to contain an anti-ligand antibody;
   (c) culturing the B cells, optionally assaying a sample of the B cells for the biological activity of the antibody, and
      (i) isolating the monoclonal antibody; or
      (ii) obtaining the immunoglobulin gene repertoire for at least the variable region of said antibody from said B cells or memory B cells; and
   (d) using said repertoire to express said antibody or a ligand-binding fragment, synthetic or biotechnological derivative thereof in a host cell and isolating the antibody or a ligand-binding fragment, synthetic or biotechnological derivative thereof;
   (e) assaying the biological activity of a sample of the antibody or a ligand-binding fragment, synthetic or biotechnological derivative thereof; optionally further comprising the step of
   (f) admixing the isolated antibody or a ligand-binding fragment, synthetic or biotechnological derivative thereof with a pharmaceutically acceptable carrier;
   characterized in that the ligand is a ligand as defined in any one of the preceding items [21] to [34] and assaying the sample and antibody, respectively, in step (a), (c), and/or (e) is performed according to the assay of any one of [31] to [34].

The recombinant production of the antibody or a ligand-binding fragment, synthetic or biotechnological derivative thereof can be performed by methods well known in the art. Optionally, the immunoglobulin gene repertoire, i.e. DNA encoding at least the variable region of said anti-ligand antibody is manipulated in order to introduce restriction sites, to change codon usage, introduce coding sequences for functional regions or peptide linkers; and/or to add or optimize transcription and/or translation regulatory sequences. RT-PCR of single sorted cells is preferably employed for obtaining the immunoglobulin gene repertoire for said antibody. A method of obtaining human antibodies using inter alia single cell RT-PCR is described for example in the international application WO2008/110372, the disclosure content of which is incorporated herein by reference, in particular the Supplementary Methods section and Example 2. As used herein, the terms “cDNA” and “mRNA” encompass all forms of nucleic acid, including but not limited to genomic DNA, cDNA, and mRNA. Cloning and heterologous expression of the antibody or antibody fragment can be performed using conventional techniques of molecular biology and recombinant DNA, which are within the skill of the art (Wrammert et al., Nature 453 (2008), 667-671 and Meijer et al., J. Mol. Bio. 358 (2006), 764-772). Such techniques are explained fully in the literature, for example in Sambrook, 1989 Molecular Cloning; A Laboratory Manual, Second Edition. For retrieval of VH/VL sequences and expression the method of Tiller et al., in J. Immunol. Methods 329 (2008), 112-124 can be used. Any appropriate host cell for expressing the recombinant human antibody may be used, e.g., a yeast, a plant cell or an animal cell. Preferably, mammalian host cells such CHO cells and HEK cells are used; see also, e.g., European patent EP 1 974 020 B1 in sections [0164] to the disclosure content of which is incorporated herein by reference.

In one embodiment the constant region of the antibody of the present invention or part thereof, in particular the CH2 and/or CH3 domain but optionally also the CH1 domain is heterologous to the variable region of the native human monoclonal antibody isolated in accordance with the method of the present invention. In this context, the heterologous constant region(s) are preferably of human origin in case of therapeutic applications of the antibody of the present invention but could also be of for example rodent origin in case of animal studies.

With respect to the use of subject mammals, in particular human patients suffering from an autoimmune and/or inflammatory disorder the candidate antibody is preferably isolated from mammals, in particular humans, which are affected with an impaired central and/or peripheral tolerance or loss of self-tolerance which may be due to or associated with a disrupted or deregulated genesis of self-tolerance, preferably caused by a monogenic autoimmune disorder. Examples of animals which provide a particularly suitable source for autoantibodies in accordance with the present invention are mammals, e.g., humans having a disorder associated with a mutation in the AIRE (Autoimmune Regulator) gene such as Autoimmune polyendocrinopathy syndrome type 1 (APS1) and autoimmune polyendocrinopathy-candidiasis-ectodermal dystrophy (APECED), respectively, (Peterson et al. Nat. Rev. Immunol. 8 (2008), 948-957), Autoimmune polyendocrinopathy syndrome type 2 (APS2) (Baker et al. J. Clin. Endocrinol. Metab. 95 (2010), E263-E270) and immunodysregulation polyendocrinopathy enteropathy X-linked syndrome (IPEX) (Powell et al. J. Pediatr. 100 (1982), 731-737; Ochs et al. Immunol. Rev. 203 (2005), 156-164). Preferably, the subject mammal from which the candidate antibodies are isolated are displaying seroreactivity against the predetermined human cytokine. For further details concerning APECED/APS1 patients and the screening of their auto-immunosome; see the description of applicant's international application WO2013/098419, the disclosure content of which is incorporated herein by reference in its entirety, and the Examples described therein, in particular the Material and Methods section on pages 112-117; Example 1 on pages 117-118 and Example 7 on page 128 and the following Tables 1 to 14; and Example 17 on pages 168-171, the disclosure content of which is incorporated herein by reference.

In this context, it is noted that though as mentioned in principle the generation of human antibodies has been reported for some antigen classes such as amyloid-beta and viral antigens, the provision of isolated and recombinant human anti-cytokine antibodies which matured in the human body does not seem to have been reported yet. Therefore, in accordance with the present invention human candidate anti-human cytokine antibodies are preferably cloned by a novel and proprietary method of isolating human antibodies, which is disclosed in applicant's international application WO2013/098420, the disclosure content of which is incorporated herein by reference in its entirety. Briefly, the sample for isolating the antibody of interest comprises or consists of peripheral blood mononuclear cells (PBMC) and serum for the detection of possible antibody reactivities. The sample derived from the subject may either be directly used for, e.g., testing seroreactivity against one or more of the desired antigen(s) or may be further processed, for example enriched for B lymphocytes. In particular, it is preferred that the sample comprises or is derived from B cells that produce the antibody of interest, most preferably memory B-cells. The memory B cells are cultured under conditions allowing only a definite life span of the B cells, typically no more than 1 to 2 weeks until singling out the cells from B cell cultures which are reactive against the desired antigen subsequently followed by RT-PCR of single sorted cells for obtaining the immunoglobulin gene repertoire; see for detailed description Examples 1 and 2 on pages 118 to 120 of international application WO2013/098419 and in particular Examples 1 to 4 on pages 27 to 31 as well as FIGS. 1 and 6 of international application WO2013/098420, the disclosure content of which is incorporated herein by reference.

Indeed, with respect to the use of patients suffering from an autoimmune and/or inflammatory disorder such as APECED it turned out that the method disclosed in applicant's international application WO2013/098419 and WO2013/098420 is particularly suitable for isolating anti-human cytokine antibodies.

The isolated antibodies of the present invention may of course not be applied as such to a patient, but usually have to be pharmaceutically formulated to ensure, e.g., their stability, acceptability and bioavailability in the patient. Therefore, in one embodiment, the method of the present invention further comprises the step of admixing the isolated and validated candidate antibody or cytokine-binding fragment thereof with a pharmaceutically acceptable carrier. A thorough discussion of pharmaceutically acceptable carriers is available in Remington's Pharmaceutical Sciences (Mack Publishing Company, N.J. 1991) and in Gennaro (2000) Remington: The Science and Practice of Pharmacy, 20^{th} edition, ISBN: 0683306472. Preferred forms for administration include forms suitable for parenteral administration, e.g. by injection or infusion, for example by bolus injection or continuous infusion. Where the product is for injection or infusion, it may take the form of a suspension, solution or emulsion in an oily or aqueous vehicle and it may contain agents commonly used in pharmaceutical formulations, such as suspending, preservative, stabilizing and/or dispersing agents. Alternatively, the antibody molecule may be in dry form, for reconstitution before use with an appropriate sterile liquid. Once formulated, the compositions can be administered directly to the subject. It is preferred that the compositions are adapted for administration to human subjects. The pharmaceutical compositions may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes.

The antibodies of the present invention or fragments thereof may be directly used as a therapeutic agent. However, in one embodiment the human anti-human cytokine antibody or cytokine binding fragment thereof which is provided by the present invention, is detectably labeled or attached to a drug, for example wherein the detectable label is selected from the group consisting of an enzyme, a radioisotope, a fluorophore and a heavy metal. Labeled human anti-human cytokine antibody or cytokine binding fragment of the present invention may be used to detect specific targets in-vivo or in-vitro including “immunochemistry/immunolabelling” like assays in-vitro. In-vivo they may be used in a manner similar to nuclear medicine imaging techniques to detect tissues, cells, or other material expressing the antigen of interest. Labels, their use in diagnostics and their coupling to the human cytokine binding molecules of the present invention are known to the person skilled in the art. In this context, in one embodiment the term pharmaceutical use includes diagnostic use in-vivo, in particular in-vivo imaging in humans.

Hence, the present invention also relates to a method of preparing a human anti-human cytokine antibody or cytokine binding fragment thereof for pharmaceutical use or as target for therapeutic intervention in the treatment of any one of the above-identified autoimmune and/or inflammatory disorders and diseases, comprising the steps of any of the above-described methods of the present invention, optionally wherein the human anti-human cytokine antibody or cytokine binding fragment thereof is detectably labeled or attached to a functional region or drug, preferably wherein the detectable label is selected from the group consisting of an enzyme, radioisotope, a fluorophore and a heavy metal.

Of course, the person skilled in the art will appreciate that the method and assay of the present invention as described hereinbefore and in the Examples can generally be applied for validating a batch of a drug which interferes with ligand-binding, for example for quality check and selecting those batches for pharmaceutical use, which prove to be sufficiently active in the assay of the present invention, i.e. effectively antagonizing or agonizing ligand-binding. Accordingly, in a further embodiment, the present invention relates to:
[36]A method for preparing a pharmaceutical composition comprising an anti-ligand antibody or ligand-binding fragment, synthetic or biotechnological variant thereof for use in the treatment of an autoimmune disorder, inflammatory disorder, cancer or a disorder mediated by a ligand-binding to a receptor comprising:
   (a) providing an anti-ligand antibody;
   (b) subjecting the antibody to the assay of any one of [31] to [34]; and
   (c) formulating the antibody or a derivative thereof in a pharmaceutical composition only if the antibody is determined to be an antagonist or agonist.

The present invention also contemplates that the reagents for use in the methods and assays described herein be contained in the form of a kit. Such kits would include, contained within suitable packaging material, a first binding reagent and a reporter, either separate or linked and a second binding reagent, both as described above. The kits may optionally further contain other components, such as instructional material for use of the kit, reagents for detecting the reporter of the first binding reagent (e.g., buffers, compounds that produce light when contacted with the detectable enzyme, etc.), a positive control for comparative or calibration purposes, etc.

Thus, in a further embodiment the present invention relates to:
[37] A kit useful for conducting a method according to any one of [21] to [30] or the assay of any one of [31] to [34], the kit comprising
   (a) a sample containing a ligand of interest (3) labeled with a reporter (4) and/or a host (reporter) cell capable of expressing and preferably secreting a ligand-reporter fusion protein or means for the preparation therefor;
   (b) a target cell (1) expressing the ligand-binding domain optionally comprising an intracellular functional region (2) or means for the preparation therefor;
   (c) means (6) for determining reporter activity (5);
   the kit preferably comprising instructions for performing the method and assay, respectively.

Appropriate target and host cells, vectors, ligands, receptors and reporters have been described hereinbefore and in the Examples. Plasmids for Renilla luciferase fusions have been described previously, e.g., in Burbello et al. BMC Biotechnol. 5 (2005), 22 and international application WO2006/071970; see also the pRL Renilla Luciferase Reporter vectors and corresponding technical bulletin for use of such reporter vectors offered by Promega Corporation, 2800 Woods Hollow Road, Madison, Wis. 53711 USA.

Furthermore, the reporter detection kits offered by InvivoGen, 5, rue Jean Rodier, F-31400 Toulouse, France, i.e. its secreted embryonic alkaline phosphatase (SEAP) reporter gene system, wherein SEAP is readily quantified by measuring the enzyme activity in the supernatant of cultured cells (like InvivoGen's Reporter Cells) using the colorimetric assays, QUANTI-Blue™ and HEK-Blue™ Detection; secreted synthetic coelenterazine luciferase Lucia reporter gene system, wherein Lucia luciferase can be directly measured in the cell culture supernatant using the ready-to-use bioluminescent assay reagent QUANTI-Luc™ and the β-Galactosidase (LacZ) reporter gene system may be adapted and used in accordance with the present invention: For example, in contrast to InvivoGen's Reporter Cells, reporter (host cell as defined above) cell lines in accordance with the present invention express and preferably secret the ligand-reporter fusion protein, e.g. a cytokine fused to SEAP or Lucia Luciferase. The second, i.e. target cell line expresses one or more ligand-binding domains, e.g. one or more cytokine receptor(s) or at least their ligand-binding domains preferably linked to a membrane anchoring polypeptide. In addition, or alternatively, the kit of the present invention comprises for one or both the reporter (host) cell and target cell corresponding expression vectors which when transfected into the appropriate cell gives rise to the desired reporter and target cell, respectively.

Thus, in a preferred embodiment, the present invention relates to:
[38] A kit useful for conducting a method and an assay, respectively, according to any one of [21] to [37], the kit comprising
   (a) at least one first expression vector comprising a DNA encoding the recombinant fusion protein of [6] or a DNA sequence encoding the reporter and an insertion site for a DNA encoding a ligand of interest or fragment thereof; and/or
   (b) optionally at least one second expression vector comprising a DNA encoding one or more ligand-binding domain(s) of the ligand of interest;
   (c) at least one third expression vector comprising a DNA encoding the reporter alone used as a positive control;
   (d) at least one control antibody which neutralizes the reporter of step (c), or alternatively unlabeled recombinant ligand as a competitive inhibitor of reporter binding;
   (e) at least one dilution buffer;
   (f) at least one assay buffer;
   (g) at least one staining solution comprising the reporter substrate;
   (h) optionally a compound that is a spectral shifter that shifts the spectrum of light generated by the bioluminescence generating system, and optionally means for the detection of the presence or activity of the reporter;
   the kit preferably comprising instructions for performing the method and assay, respectively.

Associated with the kits of the present invention, e.g., within a container comprising the kit can be a notice in the form prescribed by a governmental agency regulating the manufacture, use or sale of pharmaceuticals or biological products, which notice reflects approval by the agency of manufacture, use or sale for human administration. In addition or alternatively the kit comprises reagents and/or instructions for use in appropriate diagnostic assays. The compositions, i.e. kits of the present invention are of course particularly suitable for conducting the method and assay according to the present invention, in particular applicable for the assessment of binding of a ligand of interest to a ligand-binding domain as mentioned above.

General methods in molecular and cellular biochemistry useful for diagnostic purposes can be found in such standard textbooks as Molecular Cloning: A Laboratory Manual, 3rd Ed. (Sambrook et al. Harbor Laboratory Press 2001); Short Protocols in Molecular Biology, 4th Ed. (Ausubel et al. eds. John Wiley & Sons 1999); Protein Methods (Bollag et al. John Wiley & Sons 1996). Reagents, detection means and kits for diagnostic purposes are available from commercial vendors such as Pharmacia Diagnostics, Amersham, BioRad, Stratagene, Invitrogen, and Sigma-Aldrich as well as from the sources given any one of the references cited herein, in particular patent literature.

The method and assay as well as constituents of the kit of the present invention are illustrated in the Figures and Examples that follow.

It is to be understood that any of the features disclosed in the Figures, Figure legends and the Examples individually or in combination illustrate the features in the claims and in the embodiments [21] to [38] described above. Thus, any of the features in the claims and in the corresponding embodiments above may be replaced by one or more of the specific embodiments of the corresponding features used in the Figures, Figure legends and in the Examples. This particularly applies with respect to the target and host cell for expressing a ligand-binding domain, e.g. cytokine receptor and ligand-reporter, e.g., cytokine-luciferase fusion protein and the expression vector for use thereof. In addition, it is to be understood that the present invention also relates to any of the materials and methods described in the Figures, Figure legend and in the Examples for use in any one of the embodiments of the method and assay of the present invention. For the sake of completeness, the person skilled in the art will understand that the description, Examples and Figures may be supplemented with the disclosure content, in particular Examples and Figures of European patent applications EP 14 175 585.0 and EP 14 175 673.4 which relate to the same invention as disclosed in the present application, and from which the present application claims priority.

Further embodiments of the present invention will be apparent from the description and Examples that follow. When doing so, and if not indicated otherwise the terms “monoclonal antibody”, “mAb”, “MAB” and “MAb” are used interchangeably herein. Furthermore, while the invention is illustrated and described by way of reference to the human-derived antibody originally obtained in the experiments performed in accordance with the present invention and described in the Examples it is to be understood that the antibody or antibody fragment of the present invention include synthetic and biotechnological derivatives of an antibody which means any engineered antibody or antibody-like IL-20 binding molecule, synthesized by chemical or recombinant techniques, which retains one or more of the functional properties of the subject antibody, in particular its neutralizing activity towards IL-20. Thus, while the present invention may be described for the sake of conciseness by way of reference to an antibody, unless stated otherwise synthetic and biotechnological derivatives thereof as well as equivalent IL-20 binding molecules are meant as well and included with the meaning of the term antibody.

### BRIEF DESCRIPTION OF THE DRAWINGS

FIG. 1: Amino acid sequences of the variable region, i.e. heavy chain and kappa/lambda light chain (VH, VL) of IL-20 specific human antibodies of the present invention. A: antibody 20A10 (IgG4, kappa); B: antibody 2A11 (IgG1, lambda); C: antibody 7D1 (IgG1, lambda); D: antibody 6E11 (IgG1, lambda). Framework (FR) and complementarity determining regions (CDRs) are indicated with the CDRs being underlined. Italic amino acids indicate sequences which have not been sequenced but obtained from database.

FIG. 2: Comparison of IL-20 LIPS seroreactivities in sera isolated from APS1 patients. The individual patient is indicated on the X-axis and the RLU measurements of MABs binding on the Y-axis. Serum of patients APS1-2 and APS1-9 show enhanced IL-20 reactivity.

FIG. 3: EC 50 ELISA determination of binding of exemplary anti-IL-20 antibodies to IL-20 constructs fused either N-terminally (g1 IL-20) or C-terminally (g2 IL-20) to Gaussia luciferase. A: 2A11; B: 6E11; C: 6H2; D: 7D1; E: 20A10; F: A non-IL20 specific monoclonal antibody is used as control for non-specific binding. Antibodies 2A11, 6E11 and 20A10 bind with a high affinity to both IL-20 constructs, in contrast remaining antibodies 6H2 and 7D1 bind with a lower affinity to g2 IL-20.

FIG. 4: Human-derived anti-IL-20 monoclonal antibodies neutralize recombinant IL-20-mediated STAT3 activation in HEK 293T MSR cells transiently expressing Type I and Type II IL-20 receptors. Either cells were left untreated or stimulated with recombinant human or murine IL-20 in the absence of antibodies or in the presence of human-derived IL-20 monoclonal antibodies or a human control IgG as indicated (huIgG). Cell lysates were subjected to SDS-PAGE and pSTAT3 levels were visualized in Western blots. Total STAT3, alpha-tubulin and IL-20RB levels serve as loading control. Antibody concentration: 5 μg/ml. A: Control: rhIL-20 and rmIL-20 induce the dose-dependent phosphorylation of STAT3 in HEK 293T MSR cells transiently expressing Type I or Type II IL-20 receptors. Detection of total and phosphorylated STAT3 and IL-20RB-Myc-DDK levels. B: Stimulation with rhIL-20 (10 ng/ml) or rmIL-20 (25 ng/ml). Exemplary antibodies 2A11, 20A10 and 7D1 neutralize rhIL-20, whereas only 20A10 also potently neutralizes rmIL-20.

FIG. 5: Human-derived anti-IL-20 monoclonal antibodies neutralize rhIL-20-mediated induction of KZ136-NLuc reporter constructs in HEK 293T MSR cells transiently transfected with IL-20 receptors and KZ136-NLuc. Cells were stimulated with 120 ng/ml rhIL-20 or 200 ng/ml rmIL-20 in the presence of human-derived IL-20 monoclonal antibodies or of a control human IgG (huIgG) as indicated. A: Schematic representation of the KZ136-NLuc reporter construct. B: Control: rhIL-20 and rmIL-20 induce KZ136-NLuc reporter constructs in HEK 293T MSR cells transiently transfected with Type I IL-20 receptors and KZ136-NLuc. C, D: rhIL-20 IC 50 analysis of exemplary antibodies 20A10, 7D1 and 2A11. E: rmIL-20 IC 50 analysis of exemplary antibody 20A10 and of a control human IgG. F: IC 50 values of exemplary antibodies 20A10, 7D1 and 2A11.

FIG. 6: IC 50 analysis of human-derived anti-IL-20 monoclonal antibody 20A10 against rmIL-20 in the pSTAT3 Western blot assay. A: HEK 293T MSR cells transiently expressing Type I IL-20 receptor were either left untreated or stimulated with 25 ng/ml rmIL-20 in the absence of antibodies (−) or in the presence (+) of exemplary antibody 20A10 as indicated. Cell lysates were subjected to SDS-PAGE and pSTAT3 levels were visualized in Western blots. Total STAT3 levels serve as loading control. B: IC50 value of exemplary antibody 20A10 against rmIL-20 in the pSTAT3 Western blot assay.

FIG. 7: New assay for assessing the binding of a ligand of interest to cells expressing the relevant receptor for the ligand. The cellular binding of the ligand is measured by cell-associated luciferase activity, as the ligand of interest is used as a luciferase fusion protein. This assay also permits the assessment if antibodies directed against the ligand prevent its ability to bind to the respective receptor on the cells. Human-derived anti-IL-20 monoclonal antibodies neutralize binding of IL-20-Gaussia luciferase fusion proteins to HEK 293T MSR cells transiently expressing Type I and Type II IL-20 receptors. Cells were incubated with supernatants of HEK 293T cells expressing IL-20-Gaussia luciferase fusion proteins in the absence of antibodies (−) or in the presence of human-derived IL-20 monoclonal antibodies, a human control IgG (huIgG) or 1 μg/ml rhIL-20 as indicated. Antibody concentration: 5 μg/ml. A: Schematic representation describing a new assay for assessing the binding of a ligand of interest to cells expressing the relevant receptor for the ligand. The cellular binding of the ligand is measured by cell-associated reporter, e.g. luciferase activity, as the ligand of interest is used as a reporter, i.e. luciferase fusion protein. After removal of unbound fusion proteins a luciferase substrate is added and light emission is recorded. The light output is proportional to the amount of bound fusion protein B: This luciferase-based chemiluminescent cellular binding assay also permits the assessment if test substances such as antibodies directed against the ligand prevent its ability to bind to the respective receptor on the cells.

### REFERENCE SIGNS

1: target cell expressing a ligand-binding domain
2: ligand-binding domain such as a cytokine receptor, e.g. Type I or Type II IL-20 receptors
3: ligand such as a cytokine, e.g. IL-20
4: reporter such as an enzyme, e.g. human IL-20-Gaussia luciferase fusion protein
5: optionally substrate for the reporter such as for a luciferase
6: detectable reporter activity such as light emission
7: test substance such as an antibody, e.g. IL-20 antibody

A substrate for the reporter may not be mandatory for example in case the reporter is not an enzyme but a molecule detectable per se such as a heavy metal, e.g. gold, radioisotope or fluorescent protein where a change in reporter activity upon binding of the ligand to the ligand-binding domain may be, e.g., quenching of emission, change of wave-length, masking, and the like. C: Control: The IL-20-Gaussia luciferase fusion protein (g1 IL-20) is functionally active. Supernatants of HEK 293T containing IL-20-Gaussia luciferase fusion proteins activate STAT3 in HEK 293T MSR cells transiently expressing Type I and Type II IL-20 receptors. Cells were incubated with a serial dilution of g1 IL-20 supernatants or with control medium (NC). Detection of total and phosphorylated STAT3, IL-20RB-Myc-DDK, IL-20RA-Myc-DDK and IL-22RA (RA subunits) in Western blots. D: Control: The g1 IL-20 fusion protein specifically binds to HEK 293T MSR cells expressing both subunits of 11-20 receptor Type I. Binding is abrogated by unlabeled rhIL-20 (3 μg/ml). E: Control: Binding of g1 IL-20 to HEK 293T MSR cells expressing Type I IL-20 receptors is inhibited in a dose-dependent manner by rhIL-20, rmIL-20 and exemplary human-derived IL-20 monoclonal antibody 20A10. Binding is unaffected by the unrelated rhIL-32γ and a control human IgG (huIgG). F: Exemplary antibodies 2A11, 20A10 and 7D1 neutralize binding of g1 IL-20 to HEK 293T MSR cells transiently expressing Type I and Type II IL-20 receptors. Unlabeled rhIL-20 (1 μg/ml) serves as a positive control. Antibody concentration: 5 μg/ml. G: Binding of IFNA5-Gaussia luciferase-fusion proteins (g1 IFNA5) to HEK 293T MSR cells is inhibited by unlabeled rhIFNA2 (3 μg/ml) and by exemplary human-derived monoclonal IFN antibody 19D11 (1.7 μg/ml). A human control antibody (huIgG, 15 μg/ml) shows no effect. H: Binding of g1 IFNA2, A4, A5, A6, A7, A8, A10, A14, A16, A11 and A21 fusion proteins to HEK 293T MSR cells is inhibited by exemplary human-derived monoclonal IFN antibody 19D11. Binding of g1 IFNB and W is unaffected by 19D11. Binding of all g1 IFNs is unaffected by a control human antibody (huIgG). Antibody concentration: 5 μg/ml.

FIG. 8: IC 50 analysis of human-derived anti-IL-20 monoclonal antibodies in the chemiluminescent cellular binding assay. HEK 293T MSR cells transiently expressing Type I or Type II IL-20 receptors were incubated with g1 IL-20 supernatants in the presence of human-derived IL-20 monoclonal antibodies, A: 20A10, B: 7D1 and C: 2A11. D: IC 50 values of exemplary antibodies 20A10, 7D1 and 2A11.

FIG. 9: Cross-competition. A: Experimental setup: 96 well microplates (Costar, USA) were coated with Rabbit-anti-GLuc specific antibody (NEB, E8023S) diluted 1/250 in PBS O/N at 4° C. Plates were washed with PBS-T and blocked 1 h at room temperature with PBS containing 2% BSA (Sigma, Buchs, Switzerland). 30 μl GLuc-IL-20 was added at a final concentration of 2×10⁶ LU/well and incubated for 2 h at room temperature. The chimeric antibodies are premixed with the human competitor antibodies at a final concentration of 0.5 μg/ml vs. 3 μg/ml in 30 μl PBS and added to the plates. Upon incubation for 2 h at room temperature the plates were washed with PBS-T and the binding of the human-mouse chimeric antibodies was determined using a horseradish peroxidase conjugated goat anti-mouse IgG Fc-gamma specific antibody (Jackson Immuno Research, 1:500 in 0.5% BSA-PBS) followed by measurement of the HRP activity using a TMB substrate solution (Sigma, Buchs, Switzerland). The differential binding of exemplary human-derived anti-IL-20 monoclonal antibodies of the present invention with human-mouse (hm) chimeric constructs to distinct binding sites was investigated in cross-competition experiments with B: 20A10 hm, C: 2A11 hm, D: 7D1 hm and E: 6E11 hm. F: As an IL-20 non-binding control a human antibody binding to an unrelated antigen was used. Human-derived anti-IL-20 MAbs 20A10, 2A11 and 6E11 and did not compete with any of the other human-mouse chimeric anti-IL-20 MAbs, but human 6H2 competed with binding of human-chimeric 7D1.

FIG. 10: Cross-reactivity—Determination and comparison of binding of exemplary human-derived anti-IL-20 monoclonal antibodies 6E11, 2A11 and 20A10 of the present invention to mouse and human IL-20 by GLuc-sandwich ELISA. A: Experimental setup for the test of cross-reactivity: 96 well microplates (Costar, USA) were coated with rabbit-anti-GLuc antibody (NEB, E8023S) diluted 1/250 in PBS O/N at 4° C. Plates were washed with PBS-T and blocked 1 h at room temperature with PBS containing 2% BSA (Sigma, Buchs, Switzerland). 30 μl GLuc-IL-20 was added at a final concentration of 2×10⁶ LU/well and incubated for 2 h at room temperature. The competitor antigens recombinant hIL-20 and recombinant mouse IL-20 were titrated to the antibodies to be tested (fixed concentration of 1 μg/ml) in a serial dilution ranging from 10 μg/ml to 4.6 ng/ml. 30 μl per well of the mixtures were incubated in the wells for 1.5 h at room temperature. Plates were washed with PBS-T and the binding of human IgG to the antigen of interest in presence of the competitors was determined using a horseradish peroxidase conjugated goat anti-human Fc-gamma-specific antibody (Jackson ImmunoResearch, Europe Ltd., Cambridgeshire, UK) followed by measurement of the HRP activity using a TMB substrate solution (TMB, Sigma, Buchs, Switzerland). B, C, D: Human IL-20 inhibits binding of all tested anti-IL-20 antibodies to g2 IL-20. Murine IL-20 can inhibits binding of all tested antibodies, except 6E11, to g2 IL-20.

FIG. 11: Ear inflammation assay CytoEar IL-20. Testing the effect of different IL-20 blocking antibodies of the present invention following hIL-20 induced inflammation. To induce inflammation IL-20 was injected at a concentration of 1000 ng in a volume of 20 μl which gave the optimal experimental results after titrating different concentrations of 125 ng, 250 ng, 500 ng and 1000 ng. A: Exemplary 10-day experimental timeline. B: Overview of the experimental treatment of the experimental animal groups A to M. CytoEar ear thickness measurements calculated as fold change relative to day 0 measurements than normalized to relevant PBS controls, for each cohort. C: Overview of the effect of all normalized measurements. D: Overview of the effect of all normalized measurements. E: Overview of the effect of all normalized measurements showing IL-20 results only for the sake of clarity. Treatment with antibodies 20A10 and 7D1 (significant reduction of ear thickness at days 6 to 10, respective at days 8 to 10 for 2A11) of the present invention leads to pronounced reduction of the ear thickness resulting from IL-20 injections compared to the control treatment with IgG (of IL-20 non-related binding specificity). Mean+/−SEM, ID=intradermal cytokine injections, M=Measurements—ear thickness, S=Sacrifice of the animals; ID—cytokine injections; tested antibodies 2A11, 20A10, 7D1, and the control IgG were injected at day 0 and 6 (IP). IP=intraperitoneal antibody injection, ID=intradermal ear injection.

FIG. 12: SPR analysis. A: Detailed analysis of the sensograms concerning the binding of human and mouse IL-20 to exemplary antibodies of the present invention. A 1:1 binding kinetic was observed. The antigens were injected in concentrations of 1.5 nM, 3.125 nM, 6.25 nM, 12.5 nM, 25 nM, 50 nM and 100 nM. Calculated Affinities (KD values [M]) are indicated in the diagrams. B: The plot shows the kinetic parameters derived from the fitted curves for the association (on-rate ka) and dissociation (off-rate kd) of all tested antibodies. Dashed diagonals indicate affinities (KD). C: KD values of exemplary antibodies of this invention in comparison with SPR literature value of Biotin-Streptavidin binding. The affinities towards human IL-20 of 20A0 and 2A11 are in the sub-picomolar range and in sub-nanomolar range for mouse IL-20, respectively.

FIG. 13: Mapping of the epitopes of the human anti-IL-20 antibodies of the present invention. A: Binding of antibodies of the present invention to full length antigens coupled to the microarray. The Y-axis indicated the fluorescence intensity (RFU) upon detection with a Cy5-conjugated secondary antibody. B: Primary peptide array of 18mer peptides of human IL-20 against all antibodies of the present invention C: Primary peptide array of 18mer peptides of murine IL-20 against all antibodies of the present invention. D: Primary peptide array of human IL-20 against 20A10. In the lower panel, the peptides covering the sequence from Lysine 89 to Methionine 129 are depicted. The exemplary antibody 20A10 binds specifically to peptide 20. E: Alanine-scan of the epitope of exemplary antibody 20A10 comprising residues P101 to L129. Alanine substitutions at positions D102, H103, Y104, T105, L106, R107, K108, S111, N114, S115 and F116 led to a breakdown of mAB binding.

FIG. 14: Human IL-22-Gaussia luciferase fusion proteins bind specifically to COLO 205 cells. Cells were incubated with supernatants of HEK 293T cells expressing IL-22-Gaussia luciferase fusion proteins (g2 IL-22) in the absence of inhibitors (−) or in the presence of competitive inhibitors as indicated. A: Binding of g2 IL-22 to COLO 205 cells is inhibited by unlabeled rhIL-22 and not by the unrelated rhIFNA2. COLO 205 cells were incubated with g2 IL-22-containing supernatants of HEK 293T cells in the absence of inhibitors or in the presence of unlabeled rhIL-22 or rhIFNA2 (1 μg/ml each) as indicated. Cells were either lysed or not lysed before light output was recorded. B: Binding of g2 IL-22 to COLO 205 cells is inhibited in a dose-dependent manner by exemplary human-derived IL-22 monoclonal antibody 30G1. Binding is unaffected by a control human antibody (huIgG).

FIG. 15: Antibody 26B9 transmembrane (26B9-TM) construct design. A fragment comprising the myc-epitope and the transmembrane domain of the human platelet-derived growth factor receptor (PDGFR)-beta (database accession no.uniprot P09619, amino acids 513-561) is fused to the C-terminal portion of the constant heavy domain (A). The constant region of the kappa light chain remains unaltered (B). Upon cotransfection of HEK 293T MSR cells, properly assembled IgG is detected on the cell surface by FACS analysis using anti-human-IgG-HC-FITC and anti-human-Ig kappa-APC labelled detection antibodies.

FIG. 16: Sequence of the 26B9-TM transmembrane region (SEQ ID: 67). DNA and amino acid sequences of the 26B9-TM transmembrane region comprising the myc-epitope from DNA bases 1 to 30 and the PDGFR transmembrane domain from DNA bases 34 to 183.

FIG. 17: Human IFNW-Gaussia luciferase fusion proteins bind specifically to HEK 293T MSR cells expressing a transmembrane anti-IFNW mAb. HEK 293T MSR cells were reverse-transfected with the indicated amounts of cDNA encoding a membrane-bound version of anti-IFNW mAb 26B9 (26B9-TM) or empty vector (Mock). Forty-eight hours after transfection, IFNW-Gaussia luciferase was added (g1 IFNW) and binding was analyzed in the chemiluminescent cellular binding assay. A: Control: 26B9-TM is expressed at the cell surface of transfected HEK 293T MSR cells. Surface antibody expression was analyzed 48 hours after transfection in a cell-based ELISA. B: g1 IFNW specifically binds to cells expressing 26B9-TM in the luminescent cellular binding assay.

FIG. 18: Cross-competition assay of anti-IFNW antibodies. Binding of g1 IFNW to 26B9-TM is competed dose-dependently by soluble 26B9 and by the clonally related 31B4 antibody. In contrast, binding is not affected by a control IgG or by exemplary anti-IFNW antibody 8H1.

### DETAILED DESCRIPTION OF THE INVENTION

In a first aspect, the present invention generally relates to novel molecules binding IL-20 of mammal, preferably human origin. Accordingly, the present invention relates to recombinant human-derived monoclonal anti-interleukin-20 (IL-20) antibodies, IL-20 binding fragments, synthetic and biotechnological variants thereof as well as to equivalent IL-20-binding molecules which display the immunological, e.g. IL-20 binding characteristics and/or one or more of the biological activities of any one of anti-IL-20 antibodies 20A10, 2A11, 7D1 or 6E11 illustrated in the appended Examples and Figures. In a particularly preferred embodiment of the present invention, the mentioned anti-IL-20 binding molecules are substantially non-immunogenic in humans.

As described in Examples 2 and 6 as well as illustrated in FIGS. 3 and 10 the present invention provides anti-IL-20 antibodies which selectively or preferentially bind human IL-20, e.g. antibody 20A11 and 7D1 as well as anti-IL-20 antibodies that equally bind human and murine IL-20, i.e. antibody 20A10. Thus, in one embodiment, the human monoclonal anti-IL-20 antibody, IL-20 binding fragment, synthetic or biotechnological variant thereof of the present invention is capable of binding recombinant murine IL-20 while in another embodiment it does not substantially bind murine IL-20. Both embodiments have advantages. For example, it is commonly known that for therapeutic antibody development, the lead antibody should be tested in animal models for therapeutic activity on particular diseases and for conducting toxicology studies. Here mouse is the most commonly used animal for disease models and mouse is also the most commonly used animal for preliminary screening for toxicology studies and for long term reproductive toxicology studies. Accordingly, IL-20 antibodies and like IL-20 binding molecules derived from antibody 20A10 eliminate the need for a surrogate murine antibody due to their cross-reactivity with murine IL-20.

On the other hand, anti-IL-20 antibodies and like IL-20 binding molecules derived from antibody 20A11 or 7D1 have the advantage of selectively binding human IL-20 for which reason in animal models where a condition is induced by recombinant human IL-20 such as in HuCytoMab-Assay described in applicant's co-pending international application WO2015/001047, see also Examples 2 and 3 therein, the results are not influenced by the endogenous IL-20 of the animal. Furthermore, anti-IL-20 antibodies and like IL-20 binding molecules which discriminate between human IL-20 and corresponding animal species are advantageous for diagnostic uses. Accordingly, both embodiments of the human monoclonal anti-IL-20 antibody, IL-20 binding fragment, synthetic or biotechnological variant thereof of the present invention have their merits.

In a preferred embodiment, the human monoclonal anti-IL-20 antibody or IL-20 binding fragment thereof is capable of neutralizing a biological activity of IL-20, preferably wherein the biological activity is
(a) IL-20 signaling in a cell based STAT (signal transducers and activators of transcription) activation assay, for example to be determined in accordance with Example 3;
(b) inhibition of IL-20 cytokine cell-surface receptor binding, for example to be determined in accordance with Example 4;
(c) human IL-20 mediated activation of human IL20R1/IL20R2 receptor complexes, for example to be determined in accordance with Example 5; and/or
(d) pro-inflammatory activity of human IL-20, for example to be determined in accordance with Example 7.

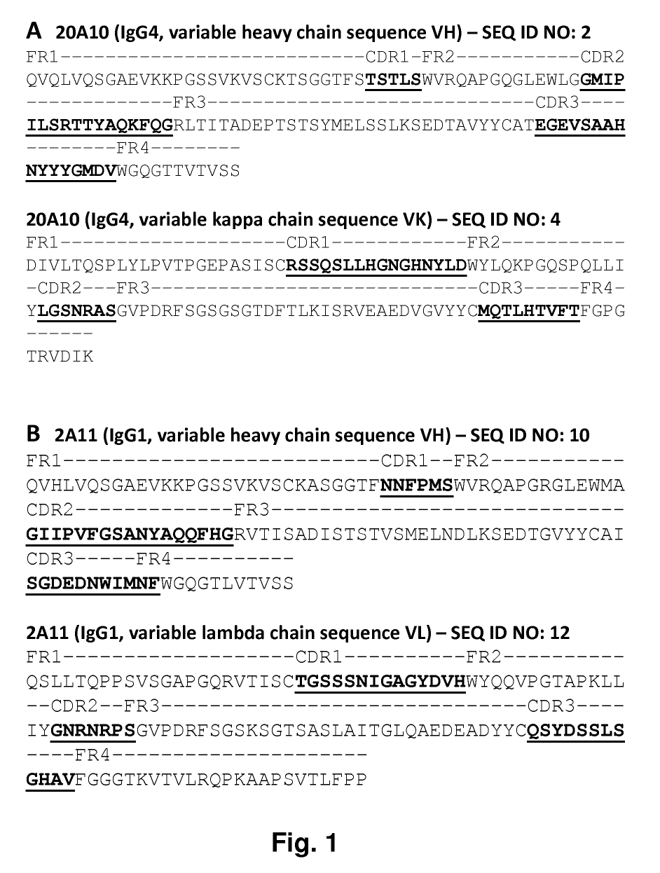

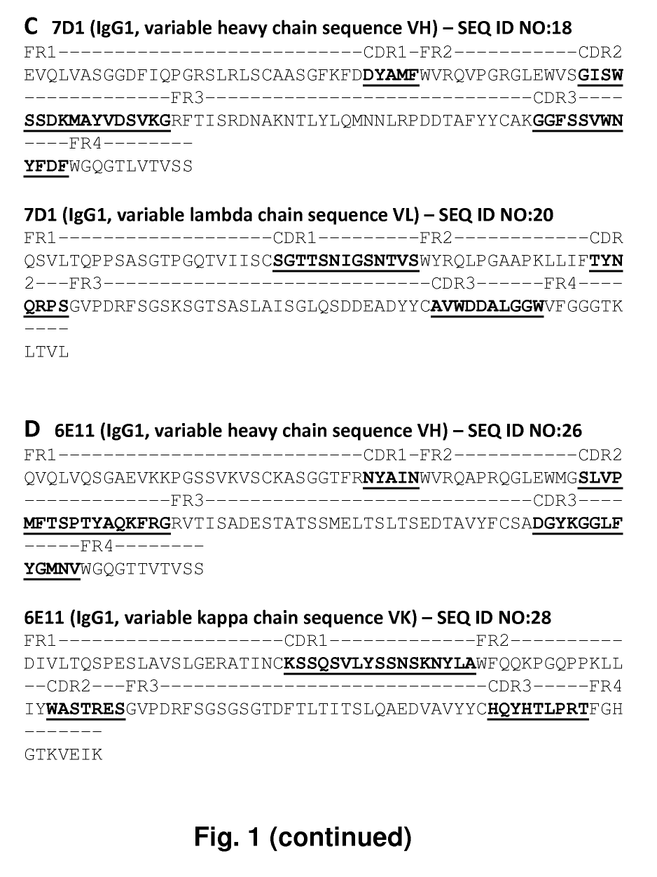

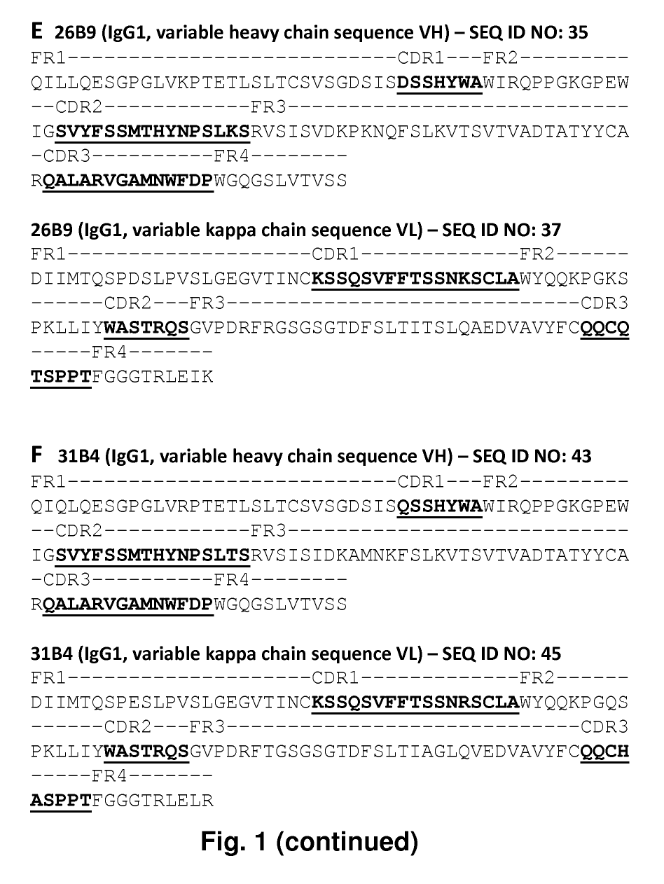

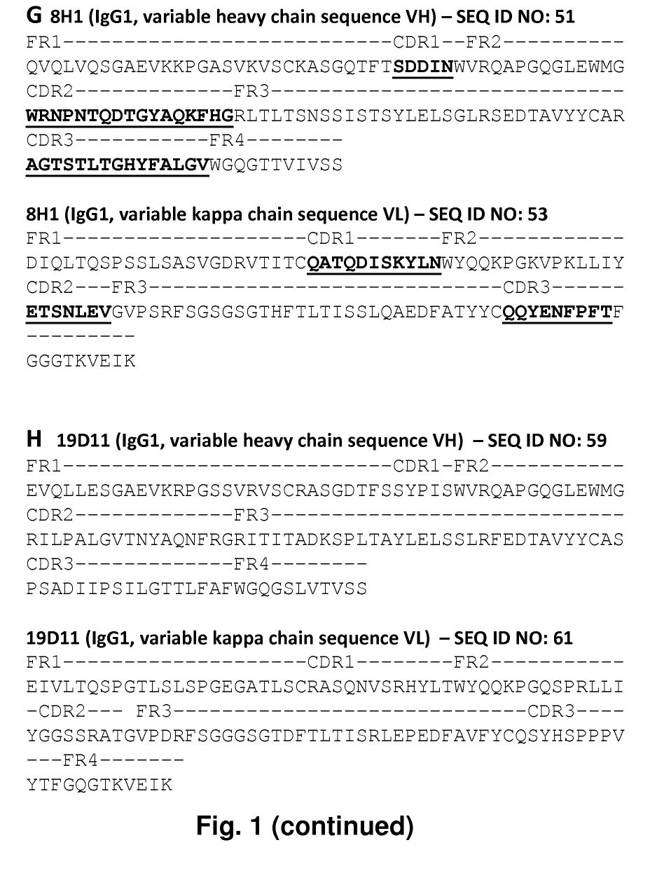

Preferably, the anti-human interleukin-20 (IL-20) antibody, an IL-20 binding fragment, synthetic or biotechnological variant thereof of the present invention comprises in its variable region:
(a) at least one complementarity determining region (CDR) of the V_{H} and/or V_{L} variable region amino acid sequences depicted in
   (i) FIG. 1 (V_{H}) (SEQ ID NOs: 2, 10, 18 and 26); and
   (ii) FIG. 1 (V_{L}) (SEQ ID NOs: 4, 12, 20 and 28);
   (b) an amino acid sequence of the V_{H} and/or V_{L} region as depicted in FIG. 1;
   (c) at least one CDR consisting of an amino acid sequence resulted from a partial alteration of any one of the amino acid sequences of (a); and
   (d) a heavy chain and/or light variable region comprising an amino acid sequence resulted from a partial alteration of the amino acid sequence of (b).

As described herein below in more detail, the anti-IL-20 antibody, IL-20 binding fragment, synthetic or biotechnological variant thereof of the present invention can be of or derived from any type, class or subclass of an immunoglobulin molecule. However, in a preferred embodiment, the antibody of the present invention is of the IgG isotype, most preferably of the IgG1 or IgG4 subclass.

In this context, the anti-IL-20 antibody, IL-20 binding fragment, synthetic or biotechnological variant thereof of the present invention preferably comprises a C_{H} and/or C_{L} constant region comprising an amino acid sequence selected from the C_{H} and C_{L} amino acid sequences set forth in Table 1 (SEQ ID NOs: 6, 8, 14, 16, 22, 24, 30 and 32) or an amino acid sequence with at least 60% identity, preferably 70% identity, more preferably 80% identity, still more preferably 90% identity, and particularly preferred at least 91%, 92%, 93%, 94%, 95%, 96%, 97%, 98% or 99%, identity to the mentioned reference sequences.

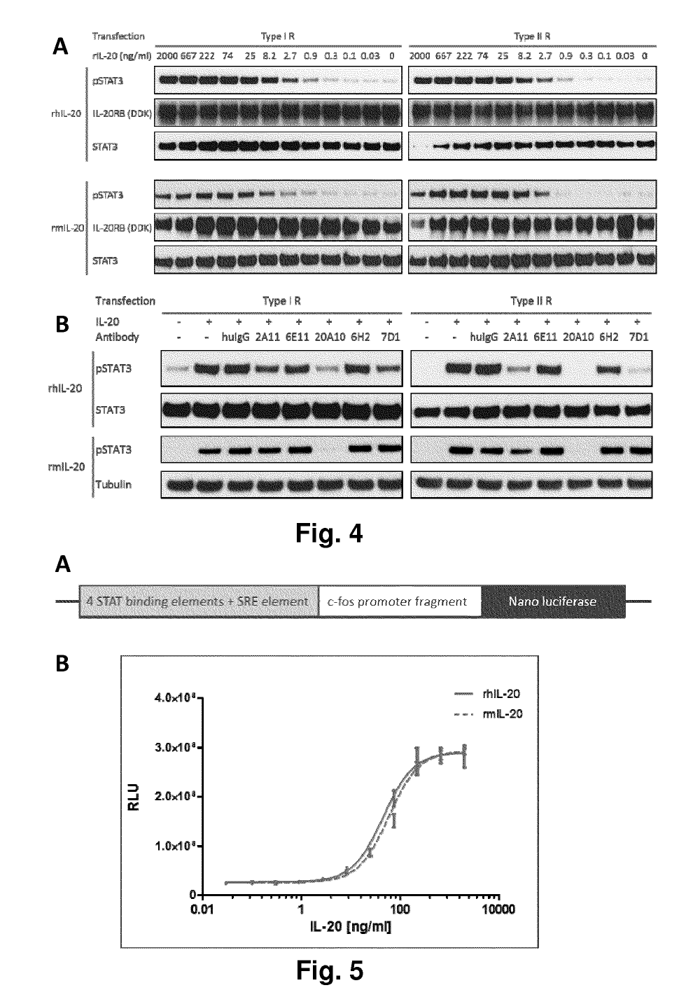

As described above, it has been found that IL-20 induces STAT3 activation either through IL-20 receptor (R) complex IL-20RA/IL-20RB (Type I) or through IL-20 receptor complex IL-22RA/IL-20RB (Type II) binding (Parish-Novak et al. J. Biol. Chem. 277 (2002), 47517-47523; Logsdon et al. Proc. Natl. Acad. Sci. USA 109 (2012), 12704-9). Furthermore, IL-20 can induce various pro-inflammatory cytokines such as monocyte chemoattractant protein (MCP)-1, IL-6 and IL-8 (see Hsieh et al. Genes Immun. 7 (2006) 2434-242), Hsu et al. Arthritis Rheum. 54 (2006), 2722-2733, supra). This activation mechanism has been used within the present invention for designing in-vitro and in-vivo assays for determining IL-20-activity, see Examples 3 and 4, as well as in a novel cellular ligand receptor binding assay described in Example 5, as well as monitoring the ear inflammation assay as described in Example 7 and FIGS. 4, 5, 6, 7E, 7F, and 8 to monitor the neutralizing properties of the antibodies of the present invention. As described in detail therein, the antibodies of the present invention have been found to have a potent neutralizing activity towards IL-20, IL-20-mediated STAT3 activation and Type I as well as Type II IL-20 receptors. Accordingly, in one embodiment the IL-20 antibody or IL-20 binding fragment thereof of the present invention is capable of reducing the biological activity of human IL-20. In a preferred embodiment, the biological activity is human IL-20 induced inflammation. Furthermore, in one embodiment of the present invention, the biological activity is determined in a STAT reporter assay and/or ear inflammation assay. Furthermore, the binding affinities of the antibodies of the present invention have been tested by ELISA and human IL-20-Gaussia luciferase fusion proteins as described herein, e.g., in Example 2 and shown in FIGS. 3 and 10. In accordance with the results of these experiments, the present invention provides several exemplary anti-IL-20 antibodies and IL-20 binding fragments thereof showing a differential binding affinity towards IL-20, which exemplify the binding and neutralization characteristics of the IL-20 binding molecules provided herein. Furthermore, as described in Example 10 and shown in FIG. 13, exemplary antibody 20A10 of the present invention has a large epitope bin, which means that a high number of amino acids in the amino acid sequence of the 18mer IL-20 peptide are essential and required for the binding of the antibody.

Accordingly, in an embodiment of the present invention an antibody or IL-20 binding fragment, synthetic or biotechnological derivative of the present invention is provided, which recognizes an IL-20 derived peptide consisting of the amino acid sequence 101-PDHYTLRKISSLANSFLT-118 (SEQ ID NO: 69), 102-DHYTLRKISSLANSF-116 (SEQ ID NO: 70) and/or 101-PDHYTLRKISSLANSFL-117 (SEQ ID No: 72), wherein only P101, 1109, S110 and/or L117 may be substituted by alanine, and the antibody does not or does not substantially recognize a peptide consisting of the amino acid sequence 97-NYQTPDHYTLRKISSLAN-114 (SEQ ID NO: 71).

Interestingly, antibody 20A10 does not bind to a corresponding peptide derived from mouse IL-20 which differs in its amino acid sequence from the human IL-20 101-118-peptide only at position Y104H and T1181, the latter being dispensable for 20A10 binding, nor to any other mouse IL-20 derived 18mer peptide, despite the capability of the antibody of binding full length mouse 11-20; see, e.g., FIG. 13. Therefore, it is tempting to assume that the 20A10 antibody recognizes a conformational epitope of human and mouse IL-20, which can also be formed or mimicked by the human IL-20 derived peptide 101-PDHYTLRKISSLANSFLT-118 (SEQ ID NO: 69) and 101-PDHYTLRKISSLANSFL-117 (SEQ ID No: 72).

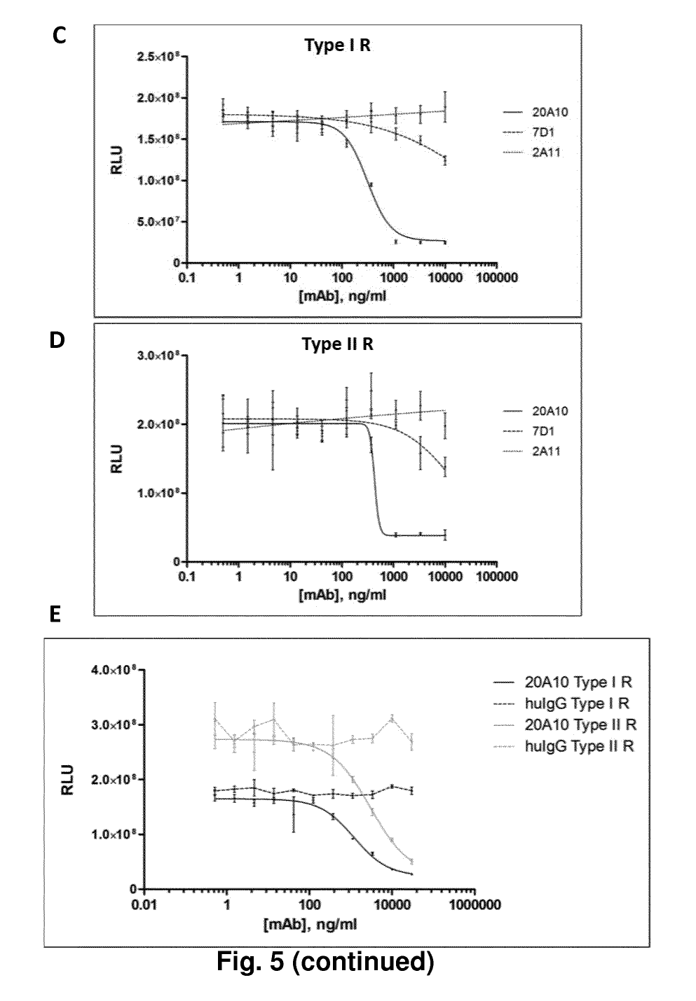

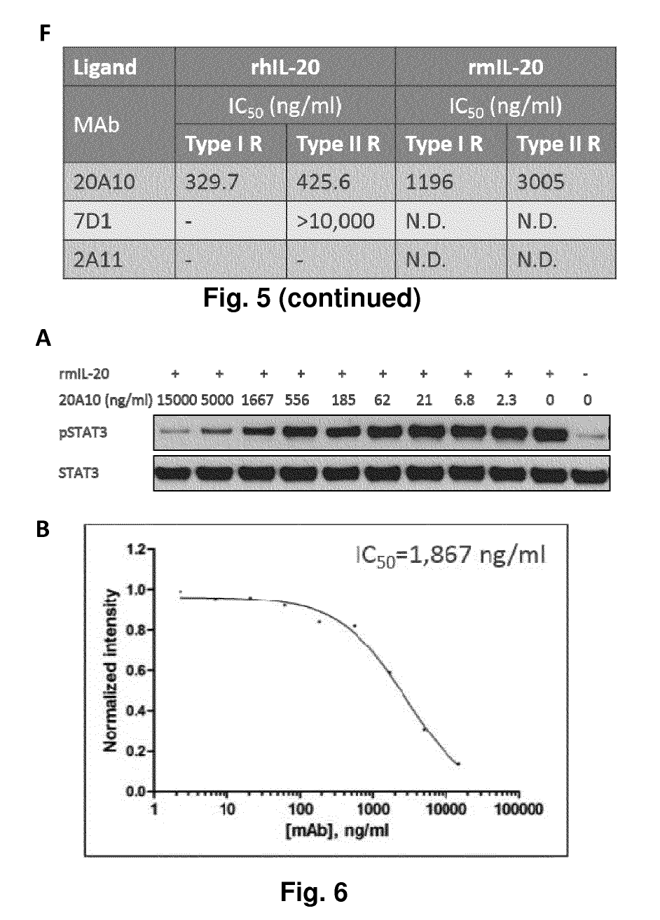

As demonstrated in the Examples and shown in the Figures anti-IL-20 antibodies illustrating the present invention are characterized by an EC and IC, respectively in the μM range up to below 10 ng (anti-IL-20 antibody 2A11). Hence, preferably, the anti-IL-20 antibody, IL-20 binding fragment, synthetic or biotechnological variant thereof of the present invention have an EC 50 as determined by ELISA assay of below about 100 ng/ml, preferably below 50 ng/ml and most preferably below 10 ng/ml for g1 IL-20 and/or g2 IL-20; see Example 2 and FIG. 3. In addition, or alternatively the anti-IL-20 antibody, IL-20 binding fragment, synthetic or biotechnological variant thereof of the present invention have an IC 50 as determined by KZ136-NLuc reporter assay of below about 1000 ng/ml, preferably below 500 ng/ml rhIL-20; see Example 3 and 4 and shown in FIG. 5F. For more details in respect of the binding affinity of the antibodies of the present invention see, e.g., section “Binding characteristics” further below.

In order to provide antibodies particularly usable for therapeutic applications, i.e. to avoid immunological responses to the antibodies of the present invention as observed for foreign antibodies, as mouse antibodies in humans (HAMA-response) the present invention preferably relates to fully human-derived antibodies since the exemplary anti-IL-20 antibodies described in the Examples illustrating the present invention have been derived from a human patient.

In this context, contrary to humanized antibodies and otherwise human-like antibodies, see also the discussion infra, the human-derived antibodies of the present invention are characterized by comprising CDRs which have been seen by human body and therefore are substantially devoid of the risk of being immunogenic. Therefore, the antibody of the present invention may still be denoted human-derived if at least one, preferably two and most preferably all three CDRs of one or both the variable light and heavy chain of the antibody are derived from the human antibodies illustrated herein.

The so human-derived antibodies may also be called “human auto-antibodies” in order to emphasize that those antibodies were indeed expressed initially by the subjects and are not in vitro selected constructs generated, for example, by means of human immunoglobulin expressing phage libraries or xenogeneic antibodies generated in a transgenic animal expressing part of the human immunoglobulin repertoire, which hitherto represented the most common method for trying to provide human-like antibodies. On the other hand, the human-derived antibody of the present invention may be denoted synthetic, recombinant, and/or biotechnological in order to distinguish it from human serum antibodies per se, which may be purified via protein A or affinity column.

However, the present invention uses and envisages further studies of the antibodies of the present invention in animal models, e.g., in transgenic mice expressing human IL-20. To avoid immunogenic effects in the experimental animals analogous to the HAMA-response in humans, in one aspect, the antibody or binding fragment of the present invention is provided, which is a chimeric antibody, preferably a chimeric rodent-human or a rodentized antibody, mostly preferred a chimeric murine-human or a murinized antibody.

In one embodiment, the anti-IL-20 antibody or IL-20 binding molecule of the present invention comprises an amino acid sequence of the V_{H} and/or V_{L} region as depicted in FIG. 1 or as encoded by the corresponding nucleic acids as indicated in Table 1. In addition, in another embodiment the present invention relates to an anti-IL-20 antibody or IL-20 binding molecule, which competes with an antibody of the present invention as defined hereinabove for specific binding to human IL-20; see Examples 6 and 7 as well as FIGS. 9-10. In particular, anti-IL-20 antibodies are provided which demonstrate the immunological binding characteristics and/or biological properties as outlined for the antibodies illustrated in the Examples and in the Figures. Where present, the term “immunological binding characteristics,” or other binding characteristics of an antibody with an antigen, in all of its grammatical forms, refers to the specificity, affinity, cross-reactivity, and other binding characteristics of an antibody.

In one embodiment, the anti-IL-20 antibody of the present invention is an antibody fragment. For example, the antibody or antibody fragment of the present invention may be selected from the group consisting of a single chain Fv fragment (scFv), an F(ab′) fragment, an F(ab) fragment, an F(ab′)₂ fragment and a single domain antibody fragment (sdAB).

A further advantage of the antibodies of the present invention is that due to the fact that the humoral immune response has been elicited against the native antigen in its physiological and cellular environment, typically autoantibodies are produced and can be isolated which recognize a conformational epitope of the antigen due to its presentation in context for example with other cellular components, presentation on a cell surface membrane and/or binding to a receptor. In contrast, conventional methods of generating monoclonal antibodies such as mouse monoclonals, humanized versions thereof or antibodies obtained from phage display typically employ an antigenic fragment of the target protein for immunizing an non-human mammal and detection, respectively, upon which usually antibodies are obtained which recognize linear epitopes or conformational epitopes limited to a two-dimensional structure of the immunogen rather than the presence of the native protein in its physiological and cellular context. Accordingly, it is prudent to expect that the autoantibodies of the present invention are unique in terms of their epitope specificity. Therefore, the present invention also relates to antibodies and like-binding molecules which display substantially the same binding specificity as the autoantibodies isolated in accordance with the method of the present invention. Such antibodies can be easily tested by for example competitive ELISA or more appropriately in a cell based neutralization assay using an autoantibody and a monoclonal derivative, respectively, thereof of the present invention as a reference antibody and the immunological tests described in the Examples or otherwise known to the person skilled in the art.

The present invention exemplifies IL-20 binding molecules, i.e. antibodies and binding fragments thereof which may be generally characterized by comprising in their variable region, i.e. binding domain at least one complementarity determining region (CDR) of the V_{H} and/or V_{L} of the variable region comprising the amino acid sequence depicted in FIG. 1 of (V_{H}) (SEQ ID NOs: 2, 10, 18 and 26) and (V_{L}) (SEQ ID NOs: 4, 12, 20 and 28)—see the exemplary CDR sequences underlined in FIG. 1 and identified in Table 1. However, as discussed in the following the person skilled in the art is well aware of the fact that in addition or alternatively CDRs may be used which differ in their amino acid sequence from those indicated in FIG. 1 by one, two, three or even more amino acid substitutions, in particular in case of CDR2 and CDR3.

As has been further demonstrated for the antibodies of the present invention, they are capable of neutralizing the biological activity of their target protein; see, e.g., the results of the STAT3 inhibition assay and ear inflammation assay described in Example 3 and FIGS. 4, 5, 6, 7E, 7F and Example 7 and FIG. 11. In this context, the term “neutralizing” means that the anti-IL-20 antibody or IL-20 binding fragment thereof of the present invention is capable of intervening with the biological activity of its target protein in a biochemical, cell-based or in-vivo assay as can be evaluated by performing the respective assay in the presence of the subject antibody of the present invention, wherein the biological activity of the target protein is reduced concomitantly with increasing level of the antibody of the present invention subjected to the assay compared to the biological activity of the protein without the presence of the antibody of the present invention and in the presence of a compound for example a control antibody which is known to leave the biological activity of the target protein unaffected in kind. Such biochemical, in-vitro and in-vivo based assay can also be performed using a reference antibody known to be capable of neutralizing the biological activity of the target protein such as has been shown for the anti-IL-20 antibodies of the present invention and subjecting the candidate antibody to the test sample, wherein either an additive neutralizing effect may be observed resulting from the combined activity of the reference and candidate antibody or a competition of the candidate antibody and reference antibody is observed which may be determined by labelling either antibody. Thus, in a preferred embodiment of the present invention, the anti-IL-20 antibody, IL-20 binding fragment, synthetic or biotechnological variant thereof of the present invention is capable of neutralizing the biological activity of its antigen. The neutralizing effect may be assessed, e.g., in the terms of the amount by which the IL-20 activity is reduced or by the time, at which such a reduction can be observed after introduction of the IL-20 binding molecules of the present invention, or, of course, in the combined terms of both.

The anti-IL-20 antibody, IL-20 binding fragment, synthetic or biotechnological variant thereof as well as immunoconjugate described further below of the present invention may be provided, as indicated in detail below, by expression in a host cell or in an in vitro cell-free translation system, for example. To express the peptide, polypeptide or fusion protein in a host cell, the nucleic acid molecule encoding the said anti-IL-20 antibody, IL-20 binding fragment, synthetic or biotechnological variant thereof as well as immunoconjugate may be inserted into an appropriate expression vector, i.e. a vector which contains the necessary elements for the transcription and translation of the inserted coding sequence. Methods, which are well known to those skilled in the art, may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al. Molecular Cloning, A Laboratory Manual (1989), and Ausubel et al. Current Protocols in Molecular Biology (1989); see also the sections “Polynucleotides” and “Expressions” further below and literature cited in the Examples section for further details in this respect.

A suitable host cell for expression of the product may be any prokaryotic or eukaryotic cell; e.g., bacterial cells such as E. coli or B. subtilis, insect cells (baculovirus), yeast cells, plant cell or an animal cell. For efficient processing, however, mammalian cells are preferred. Typical mammalian cell lines useful for this purpose include CHO cells, HEK 293 cells, COS cells and NSO cells.

The isolated antibodies of the present invention may of course not be applied as such to a patient, but usually have to be pharmaceutically formulated to ensure, e.g., their stability, acceptability and bioavailability in the patient. Therefore, in one embodiment, the method of the present invention is provided, further comprising the step of admixing the anti-IL-20 antibody, IL-20 binding fragment, synthetic or biotechnological variant thereof as well as immunoconjugates with a pharmaceutically acceptable carrier. Pharmaceutically acceptable carriers will be described in detail further below.

The present invention also relates to polynucleotides encoding at least a variable region of an immunoglobulin chain of the antibody or antigen-binding fragment of the invention. Preferably, said variable region comprises at least one complementarity determining region (CDR) of the V_{H} and/or V_{L} of the variable region as set forth in FIG. 1.

In case of a derived or equivalent sequence, said sequence shows at least 60% identity, more preferably (in the following order) at least 70%, at least 75%, at least 80%, at least 85%, at least 90%, and most preferably 95%, at least 96-99%, or even 100% identity to a sequence of the group consisting of those sequences referred to above and identified in the Sequence Listing. The percent identity between two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm, which is well known to those skilled in the art. The identities referred to herein are to be determined by using the BLAST programs as further referred to herein infra.

As mentioned above, in a preferred embodiment the present invention relates to substantially fully human antibodies, preferably IgG including at least the constant heavy chain I (C_{H}1) and the corresponding light chain of the constant region, i.e. γ-1, γ-2, γ-3 or γ-4 in combination with lambda or kappa. In a particularly preferred embodiment, the nucleotide and amino acid sequences of those constant regions isolated for the subject antibodies illustrated in the Examples are used as depicted in Table 1 below and in SEQ ID NOs: 5, 7, 13, 15, 21, 23, 29 and 31 in respect of the nucleotide sequences and/or SEQ ID NOs: 6, 8, 14, 16, 22, 24, 30 and 32 in respect of the amino acid sequences or amino acid sequences with at least 60% identity to these referenced before.

In accordance with the above, in one embodiment the present invention also provides a polynucleotide encoding at least the variable region of one immunoglobulin chain of the antibody or antigen-binding fragment of the present invention. Typically, said variable region encoded by the polynucleotide comprises at least one complementarity determining region (CDR) of the V_{H} and/or V_{L} of the variable region of the said antibody. Variable and constant regions of antibodies are described in more detail in the section “IgG structure” below. In a preferred embodiment of the present invention, the polynucleotide comprises, consists essentially of, or consists of a nucleic acid having a polynucleotide sequence encoding the V_{H} or V_{L} region of an antibody of the present invention as depicted in Table 1 below. In this respect, the person skilled in the art will readily appreciate that the polynucleotides encoding at least the variable domain of the light and/or heavy chain may encode the variable domain of either immunoglobulin chains or only one of them. In a preferred embodiment, the polynucleotide encodes the anti-IL-20 antibody or IL-20 binding fragment thereof as defined hereinabove.

TABLE 1
Nucleotide sequences of the variable and constant regions (V_{H}, V_{L}, C_{H}, C_{L}) regions
of IgG4, lambda, IL-20 specific 20A10 antibody and of IgG1, lambda, IL-20 specific
2A11, 7D1 and 6E11 antibodies of the present invention as well as nucleotide
sequences of the variable and constant regions (V_{H}, V_{L}, C_{H}, C_{L}) regions of IgG1,
kappa, IFN-α/ω specific 26B9, 31B4, 8H1 and 19D11 antibodies used in accordance
with the present invention.

| | Nucleotide and amino acid sequences of variable heavy (VH) and | |
|---|---|---|
| | variable light (VL), constant heavy (CH) and constant light (CL) | |
| Antibody | chains. | Seq ID |
| 20A10 VH | caggtgcagaggtgcaatctggggctgaggtgaagaagcctgggtcctcagtgaaggtctcctgcaag | 1 |
| DNA | acttctggaggcaccttcagcacctctactctcagctgggtgcgacaggcccctggacagggtcttgagt | |
| | ggctgggaggaatgatccctatccttagtagaacaacgtacgcgcagaagtttcagggcagactcac | |
| | gattaccgcggacgaacccacgagcacgtcctacatggaactgagcagcctgaaatctgaggacacgg | |
| | ccgtctattactgtgcgaccgagggggaagtttctgccgcgcacaactactactacggaatggacgtc | |
| | tggggccaagggaccacggtcaccgtctcctca | |
| 20A10 VH | qvqlvqsgaevkkpgssvkvscktsggtfststlswvrqapgqglewlggmipilsrttyaqkfqgrl | 2 |
| amino acid | titadeptstsymelsslksedtavyycategevsaahnvvvgmdvwgqgttvtvss | |
| 20A10 VL | gatattgtgctgactcagtctccactctacctgcccgtcacccctggagagccggcctccatctcctgcagg | 3 |
| κ DNA | tctagtcagagcctcctgcatggaaatggacacaactatttggattggtacctgcagaagccagggca | |
| | gtctccacagctcctgatctatttgggttctaatcgggcctccggggtccctgacaggttcagtggcagtg | |
| | gatcaggcacagattttacactgaaaatcagcagagtggaggctgaggatgttggggthattactgcatgc | |
| | aaactctacatactgtattcactttcggccctgggaccagagtggatatcaaa | |
| 20A10 VL | divltqsplylpvtpgepasiscrssqsllhgnghnyldwylqkpgqspqlliylgsnrasvpdrfsg | 4 |
| κ amino | sgsgtdftlkisrveaedvgvyycmqtlhtvftfgpgtrvdik | |
| acid | | |
| 20A10 CH | gcttccaccaagggcccatcggtcttccccctggcgccctgctccaggagcacctccgagagcacagcc | 5 |
| DNA | gccctgggctgcctggtcaaggactacttccccgaaccggtgacggtgtcgtggaactcaggcgccctg | |
| | accagcggcgtgcacaccttcccggctgtcctacagtcctcaggactctactccctcagcagcgtggtgac | |
| | cgtgccctccagcagcttgggcacgaagacctacacctgcaacgtagatcacaagcccagcaacaccaa | |
| | ggtggacaagagagttgagtccaaatatggtcccccatgcccatcatgcccagcacctgagttcctgggg | |
| | ggaccatcagtcttcctgttccccccaaaacccaaggacactacatgatctcccggacccctgaggtcac | |
| | gtgcgtggtggtggacgtgagccaggaagaccccgaggtccagttcaactggtacgtggatggcgtgga | |
| | ggtgcataatgccaagacaaagccgcgggaggagcagttcaacagcacgtaccgtgtggtcagcgtcct | |
| | caccgtcctgcaccaggactggctgaacggcaaggagtacaagtgcaaggtctccaacaaaggcctccc | |
| | gtcctccatcgagaaaaccatctccaaagccaaagggcagccccgagagccacaggtgtacaccctgcc | |
| | cccatcccaggaggagatgaccaagaaccaggtcagcctgacctgcctggtcaaaggcttctaccccag | |
| | cgacatcgccgtggagtgggagagcaatgggcagccggagaacaactacaagaccacgcctcccgtg | |
| | ctggactccgacggctccttcttcctctacagcaggctaaccgtggacaagagcaggtggcaggagggg | |
| | aatgtcttctcatgctccgtgatgcatgaggctctgcacaaccactacacacagaagagcctctccctgtctc | |
| | tgggtaaa | |
| 20A10 CH | astkgpsvfplapcsrstsestaalgclvkdyfpepvtvswnsgaltsgvhtfpavlqssglyslssvvtv | 6 |
| amino acid | pssslgtktytcnvdhkpsntkvdkrveskygppcpscpapeflggpsvflfppkpkdtlmisrtpevt | |
| | cvvvdvsqedpevqfnwyvdgvevhnaktkpreeqfnstyrvvsyltvlhqdwlngkeykckvsn | |
| | kglpssiektiskakgqprepqvytlppsqeemtknqvsltclvkgfypsdiavewesngqpennykt | |
| | tppvldsdgsfflysrltvdksrwqegnvfscsvmhealhnhytqkslslslgk | |
| 20A10 CL | cgaactgtggctgcaccatctgtcttcatcttcccgccatctgatgagcagttgaaatctggaactgcctctgt | 7 |
| κ DNA | tgtgtgcctgctgaataacttctatcccagagaggccaaagtacagtggaaggtggataacgccctccaat | |
| | cgggtaactcccaggagagtgtcacagagcaggacagcaaggacagcacctacagcctcagcagcac | |
| | cctgacgctgagcaaagcagactacgagaaacacaaagtctacgcctgcgaagtcacccatcagggcct | |
| | gagctcgcccgtcacaaagagcttcaacaggggagagtgt | |
| 20A10 CL | rtvaapsvfifppsdeqlksgtasvvcllnnfypreakvqwkvdnalqsgnsqesvteqdskdstysls | 8 |
| κ amino | stltlskadyekhkvyacevthqglsspvtksfnrgec | |
| acid | | |
| 2A11 VH | Caggtgcatctggttcagtctggggctgaggtgaagaagcctgggtcctcggtgaaggtacctgcaagg | 9 |
| DNA | cttctggaggcaccttcaacaattttcccatgagttgggtgcgacaggcccctggacgagggcttgagtg | |
| | gatggcagggatcatccctgtatttggttcggcaaactatgcacagcagtttcacggcagagtcacca | |
| | ttagcgcggacatatccacgagcacggtgtccatggaactgaatgacttgaaatctgaggacacgggcgt | |
| | ttattattgtgcgattagtggagatgaggacaactggatcatgaacttttggggccagggaaccctggtc | |
| | accgtctcctca | |
| 2A11 VH | qvhlvqsgaevkkpgssvkvsckasggtfnnfpmswvrqapgrglewmagiipvfgsanyaqqf | 10 |
| amino acid | hgrvtisadiststvsme1ndlksedtgvyycaisgdednwimnfwgqgtlvtvss | |
| 2A11 VL λ | cagtctttgctgacgcagccgccctcagtgtctggggccccagggcagagagtcaccatctcctgcactg | 11 |
| DNA | ggagcagctccaacatcggggcaggttatgatgtgcactggtaccagcaagttccagggacagcccc | |
| | caaactcctcatctatggaaacagaaatcggccctcaggggtccctgaccgattctctggctccaagtct | |
| | ggcacgtcagcctccctggccatcactgggctccaggctgaggatgaggctgattattactgccagtcct | |
| | atgacagcagcctgagtggtcatgcggtgttcggcggagggaccaaggtgaccgtcctacgtcagcc | |
| | caaggctgccccctcggtcactctgacccgccc | |
| 2A11 VL λ | qslltqppsvsgapgqrvtisctgsssnigagydvhwyqqvpgtapklliygnrnrpsgvpdrfsgs | 12 |
| amino acid | ksgtsaslaitglqaedeadyycqsydsslsghavfgggtkvtvlrqpkaapsvtlfpp | |
| 2A11 CH | gcctccaccaagggcccatcggtcttccccctggcaccctcctccaagagcacctctgggggcacagcg | 13 |
| DNA | gccctgggctgcctggtcaaggactacttccccgaaccggtgacggtgtcgtggaactcaggcgccctg | |
| | accagcggcgtgcacaccttcccggctgtcctacagtcctcaggactctactccctcagcagcgtggtgac | |
| | cgtgccctccagcagcttgggcacccagacctacatctgcaacgtgaatcacaagcccagcaacaccaa | |
| | ggtggacaagagagttgagcccaaatcttgtgacaaaactcacacatgcccaccgtgcccagcacctgaa | |
| | ctcctggggggaccgtcagtcttcctcttccccccaaaacccaaggacaccctcatgatctcccggacccc | |
| | tgaggtcacatgcgtggtggtggacgtgagccacgaagaccctgaggtcaagttcaactggtacgtggac | |
| | ggcgtggaggtgcataatgccaagacaaagccgcgggaggagcagtacaacagcacgtaccgtgtggt | |
| | cagcgtcctcaccgtcctgcaccaggactggctgaatggcaaggagtacaagtgcaaggtctccaacaa | |
| | agccctcccagcccccatcgagaaaaccatctccaaagccaaagggcagccccgagaaccacaggtgt | |
| | acaccctgcccccatcccgggaggagatgaccaagaaccaggtcagcctgacctgcctggtcaaaggct | |
| | tctatcccagcgacatcgccgtggagtgggagagcaatgggcagccggagaacaactacaagaccacg | |
| | cctcccgtgctggactccgacggctccacttcctctatagcaagctcaccgtggacaagagcaggtggca | |
| | gcaggggaacgtcttctcatgctccgtgatgcatgaggctctgcacaaccactacacgcagaagagcctc | |
| | tccctgtccccgggtaaa | |
| 2A11 CH | astkgpsvfplapsskstsggtaalgclvkdyfpepvtvswnsgaltsgvhtfpavlqssglyslssvvtv | 14 |
| amino acid | pssslgtqtyicnvnhkpsntkvdkrvepkscdkthtcppcpapellggpsvflfppkpkdtlmisrtp | |
| | evtcvvvdvshedpevkfnwyvdgvevhnaktkpreeqynstyrvvsvltvlhqdwlngkeykck | |
| | vsnkalpapiektiskakgqprepqvytlppsreemtknqvsltclvkgfypsdiavewesngqpen | |
| | nykttppvldsdgsfflyskltvdksrwqqgnvfscsvmhealhnhytqkslslspgk | |
| 2A11 CL λ | tcctctgaggagcttcaagccaacaaggccacactggtgtgtctcataagtgacttctacccgggagccgt | 15 |
| DNA | gacagtggcctggaaggcagatagcagccccgtcaaggcgggagtggagaccaccacaccctccaaa | |
| | caaagcaacaacaagtacgcggccagcagctacctgagcctgacgcctgagcagtggaagtcccacag | |
| | aagctacagctgccaggtcacgcatgaagggagcaccgtggagaagacagtggcccctacagaatg | |
| | ttca | |
| 2A11 CL λ | sseelqankatlvclisdfypgavtvawkadsspvkagvetttpskqsnnkyaassylsltpeqwkshr | 16 |
| amino acid | syscqvthegstvektvaptecs | |
| 7D1 VH | gaagtgcaactggtggcgtctgggggagacttcatacagcctggcaggtccctgagactctcctgtgcag | 17 |
| DNA | cctctggattcaagtttgatgattatgccatgttctgggtccggcaagttccagggaggggcctggagtgg | |
| | gtctcagggattagttggagtagtgataagatggcctatgtggactctgtgaagggccgctttaccatc | |
| | tccagagacaacgccaagaacaccctctatttgcaaatgaacaatctgagacctgacgacacggccttcta | |
| | ctactgtgcaaagggcggatttagcagtgtctggaactactttgacttctggggccagggaaccctggt | |
| | caccgtctcctca | |
| 7D1 VH | evqlvasggdfiqpgrslrlscaasgfkfddyamfwvrqvpgrglewvsgiswssdkmayvdsvk | 18 |
| amino acid | grftisrdnakntlylqmnnlrpddtafyycakggfssvwnyfdfwgqgtlvtvss | |
| 7D1 VL λ | cagtctgttctgactcaaccaccctcagcgtctgggacccccgggcagacagtcatcatctcttgttctgga | 19 |
| DNA | accacctccaacatcggcagtaatactgtaagctggtaccggcaactcccaggggcggcccccaaac | |
| | tcctcatctttacttataatcagcggccctcaggggtccctgaccgattctctggctccaagtctggcacct | |
| | ctgcctccctggccatcagtggcctccagtctgacgatgaggctgattattactgtgccgtgtgggatgac | |
| | gccctggggtggttgggtgttcggcggagggaccaagctgaccgtccta | |
| 7D1 VL λ | qsvltqppsasgtpgqtviiscsgttsnigsntvswyrqlpgaapklliftynqrpsgvpdrfsgsksgt | 20 |
| amino acid | saslaisglqsddeadyycavwddalggwvfgggtkltvl | |
| 7D1 CH | gcctccaccaagggcccatcggtcttccccctggcaccctcctccaagagcacctctgggggcacagcg | 21 |
| DNA | gccctgggctgcctggtcaaggactacttccccgaaccggtgacggtgtcgtggaactcaggcgccctg | |
| | accagcggcgtgcacaccttcccggctgtcctacagtcctcaggactctactccctcagcagcgtggtgac | |
| | cgtgccctccagcagcttgggcacccagacctacatctgcaacgtgaatcacaagcccagcaacaccaa | |
| | ggtggacaagagagttgagcccaaatcttgtgacaaaactcacacatgcccaccgtgcccagcacctgaa | |
| | ctcctggggggaccgtcagtcttcctcttccccccaaaacccaaggacaccctcatgatctcccggacccc | |
| | tgaggtcacatgcgtggtggtggacgtgagccacgaagaccctgaggtcaagttcaactggtacgtggac | |
| | ggcgtggaggtgcataatgccaagacaaagccgcgggaggagcagtacaacagcacgtaccgtgtggt | |
| | cagcgtcctcaccgtcctgcaccaggactggctgaatggcaaggagtacaagtgcaaggtctccaacaa | |
| | agccctcccagcccccatcgagaaaaccatctccaaagccaaagggcagccccgagaaccacaggtgt | |
| | acaccctgcccccatcccgggaggagatgaccaagaaccaggtcagcctgacctgcctggtcaaaggct | |
| | tctatcccagcgacatcgccgtggagtgggagagcaatgggcagccggagaacaactacaagaccacg | |
| | cctcccgtgctggactccgacggctccttcttcctctatagcaagctcaccgtggacaagagcaggtggca | |
| | gcaggggaacgtcttctcatgctccgtgatgcatgaggctctgcacaaccactacacgcagaagagcctc | |
| | tccctgtccccgggtaaa | |
| 7D1 CH | astkgpsvfplapsskstsggtaalgclvkdyfpepvtvswnsgaltsgvhtfpavlqssglyslssvvtv | 22 |
| amino acid | pssslgtqtyicnvnhkpsntkvdkrvepkscdkthtcppcpapellggpsvflfppkpkdtlmisrtp | |
| | evtcvvvdvshedpevkfnwyvdgvevhnaktkpreeqynstyrvvsvltvlhqdwlngkeykck | |
| | vsnkalpapiektiskakgqprepqvytlppsreemtknqvsltclvkgfypsdiavewesngqpen | |
| | nykttppvldsdgsfflyskltvdksrwqqgnvfscsvmhealhnhytqkslslspgk | |
| 7D1 CL λ | ggtcagcccaaggctgccccctcggtcactctgttcccgccctcctctgaggagcttcaagccaacaagg | 23 |
| DNA | ccacactggtgtgtctcataagtgacttctacccgggagccgtgacagtggcctggaaggcagatagcag | |
| | ccccgtcaaggcgggagtggagaccaccacaccctccaaacaaagcaacaacaagtacgcggccagc | |
| | agctacctgagcctgacgcctgagcagtggaagtcccacaaaagctacagctgccaggtcacgcatgaa | |
| | gggagcaccgtggagaagacagtggcccctacagaatgttca | |
| 7D1 CL λ | gqpkaapsvtlfppsseelqankatlvclisdfypgavtvawkadsspvkagvetttpskqsnnkyaas | 24 |
| amino acid | sylsltpeqwkshksyscqvthegstvektvaptecs | |
| 6E11 VH | caggtgcagctggtgcagtctggggcagaggtgaagaagcctgggtcctcagtgaaggtctcctgcaag | 25 |
| DNA | gcctctggcggcaccttccgcaactatgcgatcaactgggtgcggcaggccccgagacaaggacttga | |
| | gtggatgggaagcctcgtccctatgtttacctcaccaacgtacgcacagaagtttcggggcagagtca | |
| | cgataagcgcggacgagtccacggccacatcctccatggaactgaccagcctgacatctgaagacacgg | |
| | ccgtctacttctgttctgcagatggctacaagggcggcctcttttacggtatgaatgtctggggccaagg | |
| | gaccacggtcaccgtctcttca | |
| 6E11 VH | qvqlvqsgaevkkpgssvkvsckasggtfrnyainwvrqaprqglewmgslvpmftsptyaqkf | 26 |
| amino acid | rgrvtisadestatssmeltsltsedtavyfcsadgykgglfygmnvwgqgttvtvss | |
| 6E11 VL κ | gacatcgtgttgacccagtctccagaatccctggctgtgtctctgggcgagagggccaccatcaactgca | 27 |
| DNA | agtctagccagagtgttttatacagctccaacagtaagaactacttagcttggttccagcagaaacctg | |
| | gacagcctcctaaacttctcatttattgggcatctacccgggaatccggggtccctgatcgcttcagtggc | |
| | agcgggtctgggacagatttcactctcaccatcaccagcctgcaggctgaagatgtggcagtttattactgt | |
| | caccaatatcatactctacctcgaacgttcggccacgggaccaaggttgagatcaaa | |
| 6E11 VL κ | divltqspeslavslgeratinckssqsvlyssnsknylawfqqkpgqppklliywastresgvpdrfs | 28 |
| amino acid | gsgsgtdftltitslqaedvavyychqyhtlprtfghgtkveik | |
| 6E11 CH | gcctccaccaagggcccatcggtcttccccctggcaccctcctccaagagcacctctgggggcacagcg | 29 |
| DNA | gccctgggctgcctggtcaaggactacttccccgaaccggtgacggtgtcgtggaactcaggcgccctg | |
| | accagcggcgtgcacaccttcccggctgtcctacagtcctcaggactctactccctcagcagcgtggtg | |
| | accgtgccctccagcagcttgggcacccagacctacatctgcaacgtgaatcacaagcccagcaaca | |
| | ccaaggtggacaagaaagttcccaaatcttgtgacaaaactcacacatgcccaccgtgcccagcacc | |
| | tgaactcctggggggaccgtcagtcttcctcttccccccaaaacccaaggacaccctcatgatctcccg | |
| | gacccctgaggtcacatgcgtggtggtggacgtgagccacgaagaccctgaggtcaagttcaactggt | |
| | acgtggacggcgtggaggtgcataatgccaagacaaagccgcgggaggagcagtacaacagcac | |
| | gtaccgggtggtcagcgtcctcaccgtcctgcaccaggactggctgaatggcaaggagtacaagtgc | |
| | aaggtctccaacaaagccctcccagcccccatcgagaaaaccatctccaaagccaaagggcagccc | |
| | cgagaaccacaggtgtacaccctgcccccatcccgggatgagctgaccaagaaccaggtcagcctg | |
| | acctgcctggtcaaaggcttctatcccagcgacatcgccgtggagtgggagagcaatgggcagccgg | |
| | agaacaactacaagaccacgcctcccgtgctggactccgacggctccttcttcctctacagcaagctca | |
| | ccgtggacaagagcaggtggcagcaggggaacgtcttctcatgctccgtgatgcatgaggctctgcac | |
| | aaccactacacgcagaagagcctctccctgtctccgggtaaa | |
| 6E11 CH | astkgpsvfplapsskstsggtaalgclvkdyfpepvtvswnsgaltsgvhtfpavlqssglyslssvvtvp | 30 |
| amino acid | ssslgtqtyicnvnhkpsntkvdkkvepkscdkthtcppcpapellggpsvflfppkpkdtlmisrtpevt | |
| | cvvvdvshedpevkfnwyvdgvevhnaktkpreeqynstyrvvsvltylhqdwlngkeykckvsnkal | |
| | papiektiskakgqpreppvytlppsrdeltknqvsltclvkgfypsdiavewesngqpennykttppvl | |
| | dsdgsfflyskltvdksrwqqgnvfscsvmhealhnhytqkslslspgk | |
| 6E11 CL κ | cgaactgtggctgcaccatctgtcttcatcttcccgccatctgatgagcagttgaaatctggaactgcctctgt | 31 |
| DNA | tgtgtgcctgctgaataacttctatcccagagaggccaaagtacagtggaaggtggataacgccctccaat | |
| | cgggtaactcccaggagagtgtcacagagcaggacagcaaggacagcacctacagcctcagcagcac | |
| | cctgacgctgagcaaagcagactacgagaaacacaaagtctacgcctgcgaagtcacccatcagggcct | |
| | gagctcgcccgtcacaaagagcttcaacaggggagagtgt | |
| 6E11 CL κ | rtvaapsvfifppsdeqlksgtasvvcllnnfypreakvqwkvdnalqsgnsqesvteqdskdstysls | 32 |
| amino acid | stltlskadyekhkvyacevthqglsspvtksfnrgec | |
| 26B9 VH | cagatactactgcaggagtcgggcccaggactggtgaagcccacggagaccctgtccctcacc | 35 |
| DNA | tgtagtgtctctggtgactccatcagtgatagtagtcactactgggcctggattcgccagccccc | |
| | agggaagggaccagagtggattggcagtgtctattttagttcgatgacccactacaacccgtc | |
| | cctcaaaagtcgcgtcagcatctccgttgacaagcccaagaaccagttctccttaaaagtgacct | |
| | ctgtgactgtcgccgacacggccacatattactgtgcgagacaagcccttgcccgagtcggag | |
| | ccatgaattggttcgacccctggggccagggatctctggtcacagtctcctca | |
| 26B9 VH | qillqesgpglvkptetlsltcsvsgdsisdsshywawirqppgkgpewigsvyfssmthyn | 36 |
| amino | pslksrvsisvdkpknqfslkvtsvtvadtatyycarqalarvgamnwfdpwgqgslvtvs | |
| acid | s | |
| 26B9 VL | gacatcataatgacccagtctccagactccctgcctgtgtctctgggcgagggggtcaccatcaa | 37 |
| κ DNA | ctgcaagtccagccagagcgtctttttcacctccagtaataagagttgtttagcttggtatcag | |
| | cagaagccaggaaagtctcccaaattgctcatttactgggcatcaacccgccaatccggggtcc | |
| | ctgaccgattcagaggcagcgggtctgggacagatttctctctcaccatcaccagtctgcaggct | |
| | gaagatgtggctgtttatttctgtcagcagtgtcagacatcccctcccactttcggcggagggac | |
| | caggttggagatcaaa | |
| 26B9 VL | diimtqspdslpvslgegvtinckssqsvfftssnksclawyqqkpgkspklliywastrqs | 38 |
| κ amino | gvpdrfrgsgsgtdfsltitslqaedvavyfcqqcqtspptfgggtrleik | |
| acid | | |
| 26B9 CH | gcctccaccaagggcccatcggtcttccccctggcaccctcctccaagagcacctctgggggca | 39 |
| DNA | cagcggccctgggctgcctggtcaaggactacttccccgaaccggtgacggtgtcgtggaactc | |
| | aggcgccctgaccagcggcgtgcacaccttcccggctgtcctacagtcctcaggactctactcc | |
| | ctcagcagcgtggtgaccgtgccctccagcagcttgggcacccagacctacatctgcaacgtga | |
| | atcacaagcccagcaacaccaaggtggacaagagagttgagcccaaatcttgtgacaaaactca | |
| | cacatgcccaccgtgcccagcacctgaactcctggggggaccgtcagtcttcctcttccccccaa | |
| | aacccaaggacaccctcatgatctcccggacccctgaggtcacatgcgtggtggtggacgtgag | |
| | ccacgaagaccctgaggtcaagttcaactggtacgtggacggcgtggaggtgcataatgccaa | |
| | gacaaagccgcgggaggagcagtacaacagcacgtaccgtgtggtcagcgtcctcaccgtcct | |
| | gcaccaggactggctgaatggcaaggagtacaagtgcaaggtctccaacaaagccctcccagc | |
| | ccccatcgagaaaaccatctccaaagccaaagggcagccccgagaaccacaggtgtacaccct | |
| | gcccccatcccgggaggagatgaccaagaaccaggtcagcctgacctgcctggtcaaaggctt | |
| | ctatcccagcgacatcgccgtggagtgggagagcaatgggcagccggagaacaactacaaga | |
| | ccacgcctcccgtgctggactccgacggctccttatcctctatagcaagctcaccgtggacaag | |
| | agcaggtggcagcaggggaacgtcttctcatgctccgtgatgcatgaggctctgcacaaccact | |
| | acacgcagaagagcctctccctgtccccgggtaaatga | |
| 26B9 CH | astkgpsvfplapsskstsggtaalgclvkdyfpepvtvswnsgaltsgvhtfpavlqssglysl | 40 |
| amino | ssvvtvpssslgtqtyicnvnhkpsntkvdkrvepkscdkthtcppcpapellggpsvflfpp | |
| acid | kpkdtlmisrtpevtcvvvdvshedpevkfnwyvdgvevhnaktkpreeqynstyrvvsv | |
| | ltvlhqdwlngkeykckvsnkalpapiektiskakgqprepqvytlppsreemtknqvsltcl | |
| | vkgfypsdiavewesngqpennykttppvldsdgsfflyskltvdksrwqqgnvfscsvm | |
| | healhnhytqkslslspgk | |
| 26B9 CL | cgaactgtggctgcaccatctgtcttcatcttcccgccatctgatgagcagttgaaatctggaactg | 41 |
| κ DNA | cctctgttgtgtgcctgctgaataacttctatcccagagaggccaaagtacagtggaaggtggata | |
| | acgccctccaatcgggtaactcccaggagagtgtcacagagcaggacagcaaggacagcacc | |
| | tacagcctcagcagcaccctgacgctgagcaaagcagactacgagaaacacaaagtctacgcc | |
| | tgcgaagtcacccatcagggcctgagctcgcccgtcacaaagagcttcaacaggggagagtgtt | |
| | ag | |
| 26B9 CL | rtvaapsvfifppsdeqlksgtasvvellnnfypreakvqwkvdnalqsgnsqesvteqdsk | 42 |
| κ amino | dstyslsstltlskadyekhkvyacevthqglsspvtksfnrgec | |
| acid | | |
| 31B4 VH | cagatacagctgcaggagtcgggcccaggactggtgaggcccacggagaccctgtccctcact | 43 |
| DNA | tgtagtgtctctggtgactccatcagtcagagtagtcattactgggcctggattcgccagccccc | |
| | agggaagggaccagaatggattggcagtgtctattttagctcgatgacccactacaacccgt | |
| | ccctcacaagtcgcgtcagcatctccattgacaaggccatgaataagttctccttaaaagtgacct | |
| | ctgtgactgtcgccgacacggccacatattactgtgcgagacaggcccttgcccgagtcggag | |
| | ccatgaattggttcgacccctggggccagggatctctggtcacagtctcctca | |
| 31B4 VH | qiqlqesgpglvrptetlsltcsvsgdsisqsshywawirqppgkgpewigsvyfssmthy | 44 |
| amino | npsltsrvsisidkamnkfslkvtsvtvadtatyycarqalarvgamnwfdpwgqgslvtv | |
| acid | ss | |
| 31B4 VL | gacatcataatgacccagtctccagagtccctgcctgtgtctctgggcgagggggtcaccatcaa | 45 |
| κ DNA | ctgcaagtccagccagagcgtctttttcacctccagtaataggagttgtttagcttggtatcag | |
| | cagaagccaggacagtctcccaaattgctcatttactgggcatcaacccgccaatccggggtcc | |
| | ctgaccgattcacaggcagcgggtctgggacagatttctctctcaccatcgccggtctgcaggtt | |
| | gaagatgtggctgtttatttctgtcagcagtgtcacgcatcccctcccactttcggcggcgggac | |
| | caggttggagctcaga | |
| 31B4 VL | diimtqspeslpvslgegvtinckssqsvfftssnrsclawyqqkpgqspklliywastrqs | 46 |
| κ amino | gvpdrftgsgsgtdfsltiaglqvedvavyfcqqchaspptfgggtrlelr | |
| acid | | |
| 31B4 CH | gcctccaccaagggcccatcggtcttccccctggcaccctcctccaagagcacctctgggggca | 47 |
| DNA | cagcggccctgggctgcctggtcaaggactacttccccgaaccggtgacggtgtcgtggaactc | |
| | aggcgccctgaccagcggcgtgcacaccttcccggctgtcctacagtcctcaggactctactcc | |
| | ctcagcagcgtggtgaccgtgccctccagcagcttgggcacccagacctacatctgcaacgtga | |
| | atcacaagcccagcaacaccaaggtggacaagagagttgagcccaaatcttgtgacaaaactca | |
| | cacatgcccaccgtgcccagcacctgaactcctggggggaccgtcagtcttcctcttccccccaa | |
| | aacccaaggacaccctcatgatctcccggacccctgaggtcacatgcgtggtggtggacgtgag | |
| | ccacgaagaccctgaggtcaagttcaactggtacgtggacggcgtggaggtgcataatgccaa | |
| | gacaaagccgcgggaggagcagtacaacagcacgtaccgtgtggtcagcgtcctcaccgtcct | |
| | gcaccaggactggctgaatggcaaggagtacaagtgcaaggtctccaacaaagccctcccagc | |
| | ccccatcgagaaaaccatctccaaagccaaagggcagccccgagaaccacaggtgtacaccct | |
| | gcccccatcccgggaggagatgaccaagaaccaggtcagcctgacctgcctggtcaaaggctt | |
| | ctatcccagcgacatcgccgtggagtgggagagcaatgggcagccggagaacaactacaaga | |
| | ccacgcctcccgtgctggactccgacggctccttcttcctctatagcaagctcaccgtggacaag | |
| | agcaggtggcagcaggggaacgtcttctcatgctccgtgatgcatgaggctctgcacaaccact | |
| | acacgcagaagagcctctccctgtccccgggtaaatga | |
| 31B4 CH | astkgpsvfplapsskstsggtaalgclvkdyfpepvtvswnsgaltsgvhtfpavlqssglysl | 48 |
| amino | ssvvtvpssslgtqtyicnvnhkpsntkvdkrvepkscdkthtcppcpapellggpsvflfpp | |
| acid | kpkdtlmisrtpevtcvvvdvshedpevkfnwyvdgvevhnaktkpreeqynstyrvvsv | |
| | ltvlhqdwlngkeykckvsnkalpapiektiskakgqprepqvytlppsreemtknqvsltcl | |
| | vkgfypsdiavewesngqpennykttppvldsdgsfflyskltvdksrwqqgnyfscsvm | |
| | healhnhytqks1s1spgk | |
| 31B4 CL | cgaactgtggctgcaccatctgtcttcatcttcccgccatctgatgagcagttgaaatctggaactg | 49 |
| κ DNA | cctctgttgtgtgcctgctgaataacttctatcccagagaggccaaagtacagtggaaggtggata | |
| | acgccctccaatcgggtaactcccaggagagtgtcacagagcaggacagcaaggacagcacc | |
| | tacagcctcagcagcaccctgacgctgagcaaagcagactacgagaaacacaaagtctacgcc | |
| | tgcgaagtcacccatcagggcctgagctcgcccgtcacaaagagcttcaacaggggagagt | |
| | gttag | |
| 31B4 CL | rtvaapsvfifppsdeqlksgtasvvcllnnfypreakvqwkydnalqsgnsqesvteqdsk | 50 |
| κ amino | dstyslsstltlskadyekhkvyacevthqglsspvtksfnrgec | |
| acid | | |
| 8H1 VH | caggtgcagctggtgcagtctggggctgaggtgaagaagcctggggcctcagtgaaggtctcc | 51 |
| DNA | tgcaaggcttctggacagaccttcaccagtgatgatatcaactgggtgcgacaggcccctgga | |
| | caggggctagagtggatgggatggaggaaccctaacactcaggacacgggctatgcacag | |
| | aagttccacggcagactcaccttgaccagcaacagttccataagtacatcctatctggagttgag | |
| | cggcctgagatctgaggacacggccgtgtattactgtgcgagagcggggacttcgaccttgac | |
| | cggccactacttcgctttgggggtctggggccaggggaccacggtcatcgtctcctca | |
| 8H1 VH | qvqlvqsgaevkkpgasvkvsckasgqtftsddinwvrqapgqglewmgwrnpntqdt | 52 |
| amino | gyaqkfhgrltltsnssistsylelsglrsedtavyycaragtstltghyfalgvwgqgttvivss | |
| acid | | |
| 8H1 VL κ | gacatccagctgacccagtctccatcctccctgtctgcatctgtaggagacagagtcaccatcact | 53 |
| DNA | tgtcaggcgactcaggatattagcaaatatttaaattggtatcagcagaaaccagggaaagtc | |
| | cctaaactcctgatctacgaaacatccaatttggaagtaggggtcccatcaaggttcagtggaa | |
| | gtgggtctgggacacattttactctcaccatcagcagcctgcaggctgaagattttgcaacatatta | |
| | ctgtcaacagtatgagaatttcccgttcactttcggcggagggaccaaggtggagatcaaa | |
| 8H1 VL κ | diqltqspsslsasvgdrvtitcqatqdiskylnwyqqkpgkvpklliyetsnlevgvpsrfsg | 54 |
| amino | sgsgthftltisslqaedfatyycqqyenfpftfgggtkveik | |
| acid | | |
| 8H1 CH | gcctccaccaagggcccatcggtcttccccctggcaccctcctccaagagcacctctgggggca | 55 |
| DNA | cagcggccctgggctgcctggtcaaggactacttccccgaaccggtgacggtgtcgtggaactc | |
| | aggcgccctgaccagcggcgtgcacaccttcccggctgtcctacagtcctcaggactctactcc | |
| | ctcagcagcgtggtgaccgtgccctccagcagcttgggcacccagacctacatctgcaacgtga | |
| | atcacaagcccagcaacaccaaggtggacaagagagttgagcccaaatcttgtgacaaaactca | |
| | cacatgcccaccgtgcccagcacctgaactcctggggggaccgtcagtcttcctcttccccccaa | |
| | aacccaaggacaccctcatgatctcccggacccctgaggtcacatgcgtggtggtggacgtgag | |
| | ccacgaagaccctgaggtcaagttcaactggtacgtggacggcgtggaggtgcataatgccaa | |
| | gacaaagccgcgggaggagcagtacaacagcacgtaccgtgtggtcagcgtcctcaccgtcct | |
| | gcaccaggactggctgaatggcaaggagtacaagtgcaaggtctccaacaaagccctcccagc | |
| | ccccatcgagaaaaccatctccaaagccaaagggcagccccgagaaccacaggtgtacaccct | |
| | gcccccatcccgggaggagatgaccaagaaccaggtcagcctgacctgcctggtcaaaggctt | |
| | ctatcccagcgacatcgccgtggagtgggagagcaatgggcagccggagaacaactacaaga | |
| | ccacgcctcccgtgctggactccgacggctccttatcctctatagcaagctcaccgtggacaag | |
| | agcaggtggcagcaggggaacgtcttctcatgctccgtgatgcatgaggctctgcacaaccact | |
| | acacgcagaagagcctctccctgtccccgggtaaa | |
| 8H1 CH | astkgpsvfplapsskstsggtaalgclvkdyfpepvtvswnsgaltsgvhtfpavlqssglysl | 56 |
| amino | ssvvtvpssslgtqtyicnvnhkpsntkvdkrvepkscdkthtcppcpapellggpsvflfpp | |
| acid | kpkdtlmisrtpevtcvvvdvshedpevkfnwyvdgvevhnaktkpreeqynstyrvvsv | |
| | ltvlhqdwlngkeykckvsnkalpapiektiskakgqprepqvytlppsreemtknqvsltcl | |
| | vkgfypsdiavewesngqpennykttppvldsdgsfflyskltvdksrwqqgnvfscsvm | |
| | healhnhytqkslslspgk | |
| 8H1 CL κ | cgaactgtggctgcaccatctgtcttcatcttcccgccatctgatgagcagttgaaatctggaactg | 57 |
| DNA | cctctgttgtgtgcctgctgaataacttctatcccagagaggccaaagtacagtggaaggtggata | |
| | acgccctccaatcgggtaactcccaggagagtgtcacagagcaggacagcaaggacagcacc | |
| | tacagcctcagcagcaccctgacgctgagcaaagcagactacgagaaacacaaagtctacgcc | |
| | tgcgaagtcacccatcagggcctgagctcgcccgtcacaaagagcttcaacaggggagagtgtt | |
| | ag | |
| 8H1 CL κ | rtvaapsvfifppsdeqlksgtasvvellnnfypreakvqwkvdnalqsgnsqesvteqdsk | 58 |
| amino | dstyslsstltlskadyekhkvyacevthqglsspvtksfnrgec | |
| acid | | |
| 19D11 | gaggtgcagctgttggagtctggggctgaggtgaagaggcctgggtcgtcggtgagggtctcc | 59 |
| VH DNA | tgcagggcttctggagacaccttcagcagttaccctatcagttgggtgcgacaggcccctggac | |
| | aaggccttgagtggatgggaaggatcctccctgcccttggtgtcacaaactacgctcagaac | |
| | ttccggggcagaatcacgattaccgcggacaagtcgcccctcacagcctacttggaactgagta | |
| | gcctcagatttgaggacacggccgtgtattactgtgcgagtcccagtgcggacataattccttcg | |
| | attttggggacgaccctctttgccttctggggccagggaagcctggtcaccgtctcctca | |
| 19D11 | evqllesgaevkrpgssvrvscrasgdtfssypiswvrqapgqglewmgrilpalgvtnya | 60 |
| VH | qnfrgrititadkspltaylelsslrfedtavyycaspsadiipsilgttlfafwgqgslvtvss | |
| amino | | |
| acid | | |
| 19D11 | gaaattgtgttgacgcagtctccaggcaccctgtctctgtctccgggggaaggggccaccctct | 61 |
| VL κ | cctgcagggccagtcagaatgttagcagacactacttaacctggtaccagcagaaacctggc | |
| DNA | cagtctccccggctcctcatctatggtggctccagcagggccactggcgtcccagacaggttca | |
| | gtggcggtgggtctgggacagacttcactctcaccatcagcaggctggagcctgaagactttgc | |
| | agtgttttactgccagagctatcatagcccacctcctgtgtacactttcggccaggggaccaag | |
| | gtggagatcaaa | |
| 19D11 | eivltqspgtlslspgegatlscrasqnvsrhyltwyqqkpgqsprlliyggssratgvpdrfs | 62 |
| VL κ | gggsgtdftltisrlepedfavfycqsyhspppvytfgqgtkveik | |
| amino | | |
| acid | | |
| 19D11 | gcctccaccaagggcccatcggtcttccccctggcaccctcctccaagagcacctctgggggc | 63 |
| CH DNA | acagcggccctgggctgcctggtcaaggactacttccccgaaccggtgacggtgtcgtggaact | |
| | caggcgccctgaccagcggcgtgcacaccttcccggctgtcctacagtcctcaggactctactc | |
| | cctcagcagcgtggtgaccgtgccctccagcagcttgggcacccagacctacatctgcaacgtg | |
| | aatcacaagcccagcaacaccaaggtggacaagagagttgagcccaaatcttgtgacaaaactc | |
| | acacatgcccaccgtgcccagcacctgaactcctggggggaccgtcagtcttcctcttcccccca | |
| | aaacccaaggacaccctcatgatctcccggacccctgaggtcacatgcgtggtggtggacgtga | |
| | gccacgaagaccctgaggtcaagttcaactggtacgtggacggcgtggaggtgcataatgcca | |
| | agacaaagccgcgggaggagcagtacaacagcacgtaccgtgtggtcagcgtcctcaccgtc | |
| | ctgcaccaggactggctgaatggcaaggagtacaagtgcaaggtctccaacaaagccctccca | |
| | gcccccatcgagaaaaccatctccaaagccaaagggcagccccgagaaccacaggtgtacac | |
| | cctgcccccatcccgggaggagatgaccaagaaccaggtcagcctgacctgcctggtcaaagg | |
| | cttctatcccagcgacatcgccgtggagtgggagagcaatgggcagccggagaacaactacaa | |
| | gaccacgcctcccgtgctggactccgacggctccttcttcctctatagcaagctcaccgtggaca | |
| | agagcaggtggcagcaggggaacgtcttctcatgctccgtgatgcatgaggctctgcacaacca | |
| | ctacacgcagaanancctctccctgtccccgggtaaatga | |
| 19D11 | astkgpsvfplapsskstsggtaalgclvkdyfpepvtvswnsgaltsgvhdpavlqssglysl | 64 |
| CH amino | ssvvtvpssslgtqtyicnvnhkpsntkvdkrvepkscdkthtcppcpapellggpsvflfpp | |
| acid | kpkdtlmisrtpevtcvvvdvshedpevkfnwyvdgvevhnaktkpreeqynstyrvvsv | |
| | ltvlhqdwlngkeykckvsnkalpapiektiskakgqprepqvytlppsreemtknqvsltcl | |
| | vkgfypsdiavewesngqpennykttppvldsdgsfflyskltvdksrwqqgnvfscsvm | |
| | healhnhytqxxlslspgk | |
| 19D11 | cgaactgtggctgcaccatctgtcttcatcttcccgccatctgatgagcagttgaaatctggaactg | 65 |
| CL κ | cctctgttgtgtgcctgctgaataacttctatcccagagaggccaaagtacagtggaaggtggata | |
| DNA | acgccctccaatcgggtaactcccaggagagtgtcacagagcaggacagcaaggacagcacc | |
| | tacagcctcagcagcaccctgacgctgagcaaagcagactacgagaaacacaaagtctacgcc | |
| | tgcgaagtcacccatcagggcctgagctcgcccgtcacaaagagcttcaacaggggagagtgtt | |
| | ag | |
| 19D11 | rtvaapsvfifppsdeqlksgtasvvellnnfypreakvqwkydnalqsgnsqesyteqdsk | 66 |
| CL κ | dstyslsstltlskadyekhkvyacevthqglsspvtksfnrgec | |
| amino | | |
| acid | | |

Underlined, bold nucleotides or amino acids indicate the CDR coding regions in the variable chain sequence. Underlined, italic nucleotides or amino acids indicate sequences, which have not been sequenced but obtained from database. In the constant chains, such 15 regions are aligned with and tuned in accordance with the pertinent human germ line variable region sequences in the database; see, e.g., Vbase (http://vbase.mrc-cpe.cam.ac.uk) hosted by the MRC Centre for Protein Engineering (Cambridge, UK).

The person skilled in the art will readily appreciate that the variable domain of the antibody having the above-described variable domain can be used for the construction of other polypeptides or antibodies of desired specificity and biological function. Thus, the present invention also encompasses polypeptides and antibodies comprising at least one CDR of the above-described variable domain and which advantageously have substantially the same or similar binding properties as any one of the anti-IL-20 antibodies described in the appended examples. The person skilled in the art will readily appreciate that using the variable domains or CDRs described herein antibodies can be constructed according to methods known in the art, e.g., as described in European patent applications EP 0 451 216 A1 and EP 0 549 581 A1.

Furthermore, the person skilled in the art knows that binding affinity may be enhanced by making amino acid substitutions within the CDRs or within the hypervariable loops (Chothia and Lesk, J. Mol. Biol. 196 (1987), 901-917) which partially overlap with the CDRs as defined by Kabat. Thus, the present invention also relates to antibodies wherein one or more of the mentioned CDRs comprise one or more, preferably not more than two amino acid substitutions. Preferably, the antibody of the invention comprises in one or both of its immunoglobulin chains two or all three CDRs of the variable regions as set forth for V_{H} regions in SEQ ID NOs: 2, 10, 18 and 26, and for V_{L} regions in SEQ ID NOs: 4, 12, 20 and 28 or as indicated in FIG. 1. Preferably, the antibody or IL-20 binding fragment or biotechnological derivative thereof comprising any one of the mentioned CDRs with one or more amino acid alterations, e.g. substitutions displays the binding characteristics of any one of the antibodies illustrated in the Examples. In a particularly preferred embodiment such antibody or IL-20 binding fragment or biotechnological derivative thereof recognizes an IL-20 derived peptide consisting of the amino acid sequence 101-PDHYTLRKISSLANSFLT-118 (SEQ ID NO: 69) or 102-DHYTLRKIS SLANSF-116 (SEQ ID NO: 70) and/or 101-PDHYTLRKISSLANSFL-117 (SEQ ID No: 72), wherein only P101, 1109, S110 and/or L117 may be substituted by another amino acid, preferably alanine, while the other amino acid positions except T118 are invariant; see also Example 10 and FIGS. 13D and E. Hence, in this embodiment the antibody does also not or does not substantially recognize a peptide consisting of the amino acid sequence 97-NYQTPDHYTLRKISSLAN-114 (SEQ ID NO: 71) in view of the missing S115 and F116. In addition, as illustrated in Example 10 and FIG. 13C such antibody or IL-20 binding fragment or biotechnological derivative thereof preferably does not bind a corresponding 18mer peptide derived from mouse IL-20, preferably while it is capable of binding full length human and mouse IL-20. In this context, it is understood that determination of the mentioned binding characteristics of the antibody or IL-20 binding fragment or biotechnological derivative thereof of the present invention is preferably performed as described in the appended Examples, e.g. on a microarray as described in Example 10 and shown in FIG. 10.

The polynucleotide of the invention encoding the above described antibody may be, e.g., DNA, cDNA, RNA or synthetically produced DNA or RNA or a recombinantly produced chimeric nucleic acid molecule comprising any of those polynucleotides either alone or in combination. In a preferred embodiment a vector comprising the above polynucleotide is provided, optionally in combination with said polynucleotide which encodes the variable region of the other immunoglobulin chain of said antibody. Such vectors may comprise further genes such as marker genes which allow for the selection of said vector in a suitable host cell and under suitable conditions.

Preferably, the polynucleotide of the invention is operatively linked to expression control sequences allowing expression in prokaryotic or eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and optionally poly-A signals ensuring termination of transcription and stabilization of the transcript. Additional regulatory elements may include transcriptional as well as translational enhancers, and/or naturally associated or heterologous promoter regions.

In this respect, the person skilled in the art will readily appreciate that the polynucleotides encoding at least the variable domain of the light and/or heavy chain may encode the variable domains of both immunoglobulin chains or one chain only.

Likewise, said polynucleotides may be under the control of the same promoter or may be separately controlled for expression. Possible regulatory elements permitting expression in prokaryotic host cells comprise, e.g., the PL, lac, trp or tac promoter in E. coli, and examples for regulatory elements permitting expression in eukaryotic host cells are the AOX1 or GAL1 promoter in yeast or the CMV-, SV40-, RSV-promoter, CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the polynucleotide of the invention and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including a C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3 (Invitrogen), or pSPORT1 (GIBCO BRL).

Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the immunoglobulin light chains, heavy chains, light/heavy chain dimers or intact antibodies, binding fragments or other immunoglobulin forms may follow; see, Beychok, Cells of Immunoglobulin Synthesis, Academic Press, N.Y., (1979).

Furthermore, the present invention relates to vectors, particularly plasmids, cosmids, viruses and bacteriophages used conventionally in genetic engineering that comprise a polynucleotide encoding the antigen or preferably a variable domain of an immunoglobulin chain of an antibody of the invention; optionally in combination with a polynucleotide of the invention that encodes the variable domain of the other immunoglobulin chain of the antibody of the invention. Preferably, said vector is an expression vector and/or a gene transfer or targeting vector.

Expression vectors derived from viruses such as retroviruses, vaccinia virus, adeno-associated virus, herpes viruses, or bovine papilloma virus, may be used for delivery of the polynucleotides or vector of the invention into targeted cell population. Methods which are well known to those skilled in the art can be used to construct recombinant viral vectors; see, for example, the techniques described in Sambrook Molecular Cloning A Laboratory Manual, Cold Spring Harbor Laboratory (1989) N.Y. and Ausubel Current Protocols in Molecular Biology, Green Publishing Associates and Wiley Interscience, N.Y. (1994). Alternatively, the polynucleotides and vectors of the invention can be reconstituted into liposomes for delivery to target cells. The vectors containing the polynucleotides of the invention (e.g., the heavy and/or light variable domain(s) of the immunoglobulin chains encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for the transformation of other cellular hosts; see Sambrook, supra.

In respect to the above, the present invention furthermore relates to a host cell comprising said polynucleotide or vector. Said host cell may be a prokaryotic or eukaryotic cell. The polynucleotide or vector of the invention which is present in the host cell may either be integrated into the genome of the host cell or it may be maintained extrachromosomally. The host cell can be any prokaryotic or eukaryotic cell, such as a bacterial, insect, fungal, plant, animal or human cell; suitable host cells and methods for production of the antibodies of the present invention are described in more detail in the section “Host cells” below.

Using the above-mentioned host cells it is possible to produce and prepare an antibody of the present invention for, e.g., a pharmaceutical use or as a target for therapeutic intervention.

Therefore, in one embodiment, it is also an object of the present invention to provide a method for preparing an anti-IL-20 antibody or IL-20 binding fragment thereof, said method comprising
(a) culturing the cell as defined hereinabove; and
(b) isolating said antibody or IL-20 binding fragment thereof from the culture.

Accordingly, the present invention relates to a recombinant, preferably human-derived anti-IL-20 antibody and IL-20 binding fragment thereof, immunoglobulin chain(s) thereof encoded by the polynucleotide of the present invention or obtainable by the above-mentioned method for preparing an anti-IL-20 antibody or immunoglobulin chain(s) thereof.

Means and methods for the recombinant production of antibodies and mimics thereof as well as methods of screening for competing binding molecules, which may or may not be antibodies, are known in the art. However, as described herein, in particular with respect to therapeutic applications in human the antibody of the present invention is a human antibody in the sense that application of said antibody is substantially free of an immune response directed against such antibody otherwise observed for chimeric and even humanized antibodies.

The anti-IL-20 antibody, IL-20 binding fragment, synthetic or biotechnological variant thereof may be directly used as a therapeutic agent. However, in one embodiment the anti-IL-20 antibody, IL-20 binding fragment, synthetic or biotechnological variant thereof which is provided by the present invention, is detectably labeled or attached to a drug, preferably wherein the detectable label is selected, for example from the group consisting of an enzyme, a radioisotope, a fluorophore, a peptide and a heavy metal. An antibody or otherwise immunoglobulin component comprising and conjugated to, respectively, a therapeutic agent or a detectable label is also called an “immunoconjugate”.

Thus, in a further embodiment the present invention relates to an immunoconjugate comprising the human-derived monoclonal anti-human IL-20 antibody of the present invention or mentioned derivatives thereof, which preferably comprises a functional moiety such as a radionuclide, enzyme, substrate, cofactor, fluorescent marker, chemiluminescent marker, peptide tag, heavy metal, magnetic particle, drug, or a toxin. In one embodiment, the immunoconjugate of the present invention is pegylated, for example in order to improve half-life and bioavailability of the antibody in the human body. The preparation of immunoconjugates and appropriate detectable labels, drugs and toxins are known in the art and described, e.g., in international application WO2005/052000.

Labeled antibodies or antigen-binding fragments and immunoconjugates of the present invention may be used to detect specific targets in vivo or in vitro including “immunochemistry/immunolabelling” like assays in vitro. In-vivo they may be used in a manner similar to nuclear medicine imaging techniques to detect tissues, cells, or other material expressing the antigen of interest. Labels, their use in diagnostics and their coupling to the binding molecules of the present invention are described in more detail in section “labels and diagnostics” further below.

IL-20 specific antibodies identified in the present invention may be involved in severely impairing the immune system of the affected individual, which is associated with, e.g., symptoms observed in APECED patients. Therefore, it is a further aspect of the present invention, to extinguish or at least relieve the pathological reactions of subjects suffering from autoimmune disorders by providing means and measures to minimize the number of autoantibodies and/or their effects in a diseased human patient or animal. Thus, in one embodiment the present invention also relates to a peptide or peptide-based compound comprising an epitope specifically recognized by an autoantibody of the present invention. A similar effect as by application of competitive antigens, sequestering and preventing thereby the binding of the autoantibodies to their respective targets may be obtained by anti-idiotypic antibodies, as described in detail further below. Therefore, in one embodiment the present invention also provides an anti-idiotypic antibody of an autoantibody of the present invention.

As already indicated above, the present invention also relates to the anti-idiotypic antibody or the peptide or peptide-based compound of the present invention for use in the treatment of a disorder as defined above, i.e. a disorder associated with a disrupted or deregulated genesis of self-tolerance. These isolated antibodies or fragments thereof of the present invention can be used as immunogenes to generate a panel of monoclonal anti-idiotypes. For suitable methods for the generation of anti-idiotypic antibodies see Raychadhuri et al. J. Immunol. 137 (1986), 1743 and for T cells see Ertl et al. J. Exp. Med. 159 (1985), 1776. The anti-idiotypic antibodies will be characterized with respect to the expression of internal image and non-internal image idiotypes using standard assays routinely practiced in the art as described in detail by Raychaudhuri et al. J. Immunol. 137 (1986), 1743. If an anti-idiotypic antibody structurally mimics the antigen of the antibody it is binding to or bound by, it is called the “internal image” of the antigen.

Methods of providing molecules which mimic an idiotype of an autoimmune disease-associated auto-antibody (autoantibodies) are described in the art; see, e.g., international application WO03/099868, the disclosure content of which incorporated herein by reference. For example, such method may comprise the following steps: (a) providing autoantibodies in accordance with the method of the present invention; (b) binding the autoantibodies to a solid phase to form an affinity matrix; (c) contacting pooled plasma or B cells comprising immunoglobulins with the affinity matrix followed by removal of unbound plasma components; (d) eluting bound immunoglobulins, being anti-Idiotypic antibodies (anti-Id) to autoantibodies, from the matrix; (e) providing a molecular library comprising a plurality of molecule members; and (e) contacting the anti-Id with the molecular library and isolating those bound molecules which are bound by the anti-Id, the bound molecules being molecules which mimic an idiotype of autoantibodies. A method of isolating idiotypic autoantibodies in disclosed in international application WO2010/136196, the disclosure content of which incorporated herein by reference, which describes immunoglobulin preparations containing natural polyclonal IgG-reactive antibodies (Abs) isolated from normal human serum (NHS), for the treatment of autoimmune diseases and immune system disorders. The IgG-reactive Abs potently neutralize disease-associated or pathogenic autoantibodies present in sera of patients suffering from autoimmune diseases, by binding to their antigenic determinants located either within or near (e.g. overlapping with) the antigen combining sites.

The present invention also relates to compositions comprising any one of the aforementioned anti-IL-20 antibodies, IL-20 binding fragments, synthetic or biotechnological variants thereof, the polynucleotides, the vectors, the cells, the peptides or peptide-based compounds of the present invention and/or a cocktail of anti-IL-20 antibodies or IL-20 binding fragments thereof which in combination display the features of an anti-IL-20 antibody or IL-20 binding fragment thereof of the present invention. In addition or alternatively in one embodiment the composition or the kit of the present invention comprises the anti-idiotypic antibody of the present invention. In one embodiment the composition is a pharmaceutical composition and further comprises a pharmaceutically acceptable carrier. Pharmaceutically acceptable carriers, administration routes and dosage regimen can be taken from corresponding literature known to the person skilled in the art and are described as well in more detail in sections “Pharmaceutical carriers” and “Dosage regimen” further below.

In addition, the present invention relates to a process for the manufacture of a composition comprising the anti-IL-20 monoclonal antibody or a IL-20 binding fragment or biotechnological derivative thereof, which manufacture comprises the step of preparation of the antibody, IL-20 binding fragment or biotechnological derivative thereof by expression in a recombinant host organism of transforming DNA encoding the antibody, a IL-20 binding fragment or biotechnological derivative thereof. In one embodiment, the composition is a pharmaceutical composition, wherein the step of preparation of the antibody, IL-20 binding fragment or biotechnological derivative thereof is followed, optionally after one or more steps in between by admixing the antibody, IL-20 binding fragment or biotechnological derivative thereof with a pharmaceutically acceptable carrier in the manufacture of a pharmaceutical composition. For example, before formulating in the pharmaceutical composition, the antibody or IL-20 binding fragment thereof may be purified from the cell culture to pharmaceutical grade and/or derivatized, for example pegylated or conjugated to a diagnostic label or drug so as obtain the pharmaceutical composition.

Besides biochemical and cell based in vitro assays therapeutic utility of the antibodies of the present invention can be validated in appropriate animal models as described in detail in the Examples section further below.

In one embodiment the pharmaceutical composition further comprises an additional agent useful for treating an inflammation or an autoimmune disorder, preferably wherein said agent is selected from the group consisting of Non-Steroidal Anti-Inflammatory Drugs (NSAIDs), Corticosteroids, Anti-Histamines and combinations thereof. In addition or alternatively, in a further embodiment the pharmaceutical composition further comprises an additional agent useful for treating an inflammation related disease, selected from the group consisting of immunosuppressive and anti-inflammatory or “anti-rheumatic” drugs.

In another embodiment, the composition is a diagnostic composition or kit and further comprises reagents conventionally used in immuno- or nucleic acid based diagnostic methods.

Furthermore, the present invention provides the aforementioned anti-IL-20 antibody, IL-20 binding fragment, synthetic or biotechnological variant thereof, or the composition as defined hereinabove for use in a method of:
(a) treating or preventing the progression of an immune mediated or autoimmune disease or condition;
(b) amelioration of symptoms associated with an immune mediated or autoimmune disease or condition; and/or
(c) diagnosing or screening a subject for the presence or for determining a subject's risk for developing an immune mediated or autoimmune disease or condition;
   wherein the disease or condition is associated with the expression of IL-20, elevated and/or detrimental IL-20 activity in a patient.

As indicated above, due to their binding specificity, the IL-20 binding molecules of the present invention such as antibodies and fragments thereof may preferably be used in the above-defined method of treatment, amelioration, diagnosing and/or screening of an immune mediated or autoimmune disorder or condition associated with and/or caused by expression of IL-20, elevated and/or detrimental activity of IL-20.

For example, expression, elevated and/or detrimental IL-20 activity has been observed in rheumatoid arthritis (RA) synovial tissue biopsies, psoriatic skin lesions and ankylosing spondylitis (AS), wherein the level of IL-20 expression correlated positively with the severity of inflammation (Kragstrup et al. Cytokine 41 (2008), 16-23, Blumberg et al. Cell 104 (2001), 9-19, Toyhama et al. Eur. J. Immunol. 39 (2009), 2779-88, supra). Besides rheumatoid arthritis (RA), psoriasis and ankylosing spondylitis (AS), IL-20 was found functionally associated with several other disorders, e.g., atherosclerosis (Chen et al. Arterioscler. Thromb. Vasc. Biol. 26 (2006), 2090-2095, supra), acute renal failure (Li et al. Genes Immun. 9 (2008), 395-404, supra), ulcerative colitis (Fonseca-Camarillo et al. J. Clin. Immunol. 33 (2013), 640-8, supra), ischemic stroke (Chen and Chang J. Immunol. 182 (2009), 5003-12, supra) as well as osteopenia and osteoporosis (Hsu et al. J. Exp. Med. 208 (2011), 1849-1861, supra).

Also, increased transcription of IL-20 has been observed in non-small cell lung (NSCL) cancer (Baird et al. Eur. J. Cancer 47 (2011), 1908-18, supra) and in muscle invasive bladder cancer (Lee et al. PLoS One 7 (2012), e40267, supra). Expression of IL-20 and its receptor subunits was higher in clinical oral tumor tissue as well as breast cancer tissue than in non-tumorous tissue (Hsu et al. Mol. Cancer Res. 10 (2012) 1403-9, Hsu et al. J. Immunol. 188 (2012) 1981-91, all supra).

Therefore, in one embodiment the anti-IL-20 antibody or IL-20 binding fragment, synthetic or biotechnological variant thereof or the composition as defined hereinabove for use in the above-mentioned method is provided, wherein said disease is an autoimmune disease, preferably selected from the group consisting of rheumatoid arthritis (RA), ankylosing spondylitis and other forms of spondyloarthritis including but not limited to psoriatic arthritis, psoriasis, vascular inflammation and atherosclerosis, atopic dermatitis and cancer including non-small cell lung (NSCL) cancer, muscle invasive bladder cancer, oral tumors and breast cancer.

Due to the multitude of molecules suitable in treatment of, e.g., disorders associated with inflammation presented herein, the present invention also relates to methods of treatment, diagnosing and/or prognosticate the probable course and outcome of such disorders, preferably wherein the immune mediated or autoimmune disease or condition is associated with the expression, elevated and/or detrimental activity of IL-20 and to the use of the molecules of the present invention. In one embodiment a method for treating of such a disorder is provided, which method comprises administering to a subject in need thereof a therapeutically effective amount of the aforementioned anti-IL-20 antibody, IL-20 binding fragment, synthetic or biotechnological variant thereof, the cocktail of antibodies which in combination display the features of an antibody of the present invention, the anti-idiotypic antibody or the peptide or peptide-based compound.

Furthermore, in one embodiment the present invention relates to a method of treating an immune mediated or autoimmune disease or condition associated with the expression, elevated and/or detrimental activity of IL-20 comprising administering to a subject a therapeutically effective amount of a ligand binding molecule comprising:
(a) at least one CDR of the anti-IL-20 antibody or IL-20 binding fragment thereof of the present invention; or
(b) at least one anti-idiotypic antibody and/or peptide or peptide-based compound as defined hereinabove.

Treatment methods based on the use of only one monoclonal antibody specific for an epitope of a particular antigen, which is related or causing a disease may suffer from several shortcomings. For example, difficulties and probably inefficiency of treatment may stem from the multiplicity of the pathogenic mechanisms causing a specific disorder requiring targeting of several antigens simultaneously. Furthermore, the inherent diversity of the patient population has to be taken into account concerning, e.g., polymorphism, heterogeneity of glycosylation or slight denaturation of a given antigen, either in different or in one patient which may lead to a decreased binding efficiency of the monoclonal antibody used at least. Some of these shortcomings may be circumvented by, e.g., pretreatment screenings to determine whether the antigen is immunologically relevant to the patients intended to be treated and whether there are any epitope changes in the particular patients. However, such screenings are often omitted either due to treatment urgency or to cost restraints. Therefore, the present invention further relates to methods based on the application of more than one type of a binding molecule at once to a patient, i.e. to the application of a cocktail of binding molecules. These binding molecules may specifically bind to IL-20 at different epitopes, each of the binding molecules applied may bind specifically IL-20 or several binding molecules are used binding to several epitopes of IL-20. In case the binding molecules of the present invention are directed (bind specifically) towards IL-20 as antigen, their binding specificity is directed towards distinct epitopes of said antigen. The use of such cocktails is in particular envisaged for the treatment of patients suffering from autoimmune disorders such as APS1, who in view of the presence of autoantibodies against about 3000 endogenous antigens are often not amenable to monotherapy with one particular antibody. In such cases, combination therapy with two or more monoclonal antibodies and/or peptides and peptide-based compounds of the present invention with the same or different antigen specificity are expected to achieve at least some relief of the symptoms.

Therefore, in one embodiment a further method of treating a disorder is provided comprising administering to a subject a therapeutically effective amount of a cocktail consisting essentially of at least two, three, four, five and more components selected from the groups consisting of:
(a) the anti-IL-20 antibody, IL-20 binding fragment, synthetic or biotechnological variant thereof as defined hereinabove; and/or
(b) an anti-idiotypic antibody of the present invention, and/or from a peptide or peptide-based compound of the present invention, which peptide or peptide-based compound comprises an epitope specifically recognized by an antibody or antigen-binding fragment thereof of the present invention.

The present invention naturally extents also to diagnostic and prognostic methods directed towards diagnosing immune mediated or autoimmune conditions and disorders associated with expression, elevated and/or detrimental activity of IL-20, and/or prognosis of the development of the disease, i.e. its progression, response to treatment or recovery. Therefore, in one embodiment the present invention relates to a method of diagnosing an immune mediated or autoimmune disease or condition in a subject associated with the expression, elevated and/or detrimental activity of IL-20 comprising contacting a biological sample of the subject with an anti-IL-20 antibody or IL-20 binding fragment thereof of the present invention, and detecting the presence of IL-20. Furthermore, in one embodiment the present invention relates to a method of detecting or determining IL-20 in an isolated biological sample comprising admixing the sample with an anti-IL-20 antibody of the present invention, allowing the antibody to form a complex with IL-20 present in the mixture, and detecting the complex present in the mixture.

As already mentioned above, in one embodiment the present invention relates to a kit for the diagnosis of an immune mediated or autoimmune disease or condition associated with the expression of IL-20, said kit comprising the aforementioned anti-IL-20 antibody, IL-20 binding fragment, synthetic or biotechnological variant thereof, the anti-idiotypic antibody or the peptide or peptide-based compound, the polynucleotide, the vector or the cell, optionally with reagents and/or instructions for use.

Associated with the kits of the present invention, e.g., within a container comprising the kit can be a notice in the form prescribed by a governmental agency regulating the manufacture, use or sale of pharmaceuticals or biological products, which notice reflects approval by the agency of manufacture, use or sale for human administration. In addition or alternatively the kit comprises reagents and/or instructions for use in appropriate diagnostic assays. The compositions, i.e. kits of the present invention are of course particularly suitable for the diagnosis, prevention and treatment of a disorder or condition which is accompanied with the expression of IL-20, in particular applicable for the treatment of diseases as mentioned above. In a particularly preferred embodiment the disorder is associated with expression of IL-20.

In another embodiment the present invention relates to a diagnostic composition comprising any one of the above described IL-20-binding molecules, antibodies, antigen-binding fragments, peptides or peptide-based compounds, polynucleotides, vectors or cells of the invention and optionally suitable means for detection such as reagents conventionally used in immune- or nucleic acid based diagnostic methods. The antibodies of the invention are, for example, suited for use in immunoassays in which they can be utilized in liquid phase or bound to a solid phase carrier. Examples of immunoassays which can utilize the antibody of the invention are competitive and non-competitive immunoassays in either a direct or indirect format. Examples of such immunoassays are the radioimmunoassay (RIA), the sandwich (immunometric assay), flow cytometry and the Western blot assay. The antigens and antibodies of the invention can be bound to many different carriers and used to isolate cells specifically bound thereto. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention. There are many different labels and methods of labeling known to those of ordinary skill in the art. Examples of the types of labels which can be used in the present invention include enzymes, radioisotopes, colloidal metals, fluorescent compounds, chemiluminescent compounds, and bioluminescent compounds; see also the embodiments discussed hereinabove.

In this context, the present invention also relates to means specifically designed for this purpose. For example, a protein- or antibody-based array may be used, which is for example loaded with either antigens derived from IL-20 and containing the disease-associated antigen in order to detect autoantibodies which may be present in patients suffering from an autoimmune disease. Design of microarray immunoassays is summarized in Kusnezow et al. Mol. Cell Proteomics 5 (2006), 1681-1696. Accordingly, the present invention also relates to microarrays loaded with binding molecules or antigens identified in accordance with the present invention.

The present invention also makes use of and relates to embodiments of IL-20 antagonists, e.g. anti-IL-20 antibodies described in the prior art, which are applicable and useful for the human-derived monoclonal anti-human IL-20 antibody or an IL-20 binding fragment, synthetic or biotechnological variant thereof of the present invention. For example, international application WO2005/052000 describes inter alia rat anti-human IL-20 as well as anti-IL-20RA and anti-IL-20RB monoclonal antibodies including means and methods for their characterization in-vitro and in-vivo as well as uses thereof, which can also be applied in accordance with the human-derived monoclonal anti-human IL-20 antibody of the present invention and mentioned derivatives thereof.

Thus, in a further embodiment the present invention relates to an in-vitro method for reducing or inhibiting IL-20-induced proliferation or differentiation of hematopoietic cells and hematopoietic cell progenitors comprising culturing bone marrow or peripheral blood cells with a composition comprising a human-derived monoclonal anti-human IL-20 antibody of the present invention and mentioned derivatives thereof, immunoconjugate or pharmaceutical composition as disclosed herein, wherein said reduction or inhibition is measured as a proliferation or differentiation of the hematopoietic cells in the bone marrow or peripheral blood cells as compared to bone marrow or peripheral blood cells cultured in the absence of soluble cytokine. The hematopoietic cells and hematopoietic progenitor cells may be lymphoid cells, preferably the lymphoid cells are macrophages or T cells.

Hence, in principle the human-derived monoclonal anti-human IL-20 antibody of the present invention and mentioned derivatives thereof, immunoconjugate and pharmaceutical composition as disclosed herein can be applied in accordance with any of the therapeutic uses of IL-20 antagonists and anti-IL-20 antibodies in particular as described in international application WO2005/052000.

### Definitions and Embodiments

Unless otherwise stated, a term and an embodiment as used herein is given the definition as provided and used in international applications WO2013/098419 and WO2013/098420 Supplementary, a common term as used herein is given the definition as provided in the Oxford Dictionary of Biochemistry and Molecular Biology, Oxford University Press, 1997, revised 2000 and reprinted 2003, ISBN 0 19 850673 2.

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

The term “neutralizing” and “neutralizing antibody”, respectively, is used as common in the art in that an antibody is meant that reduces or abolishes at least some biological activity of an antigen or of a living microorganism. For example, an anti-IL-20 antibody of the present invention is a neutralizing antibody, if, in adequate amounts, it abolishes or reduces the activity of IL-20 for example in an assay as described in the Examples. Neutralization is commonly defined by 50% inhibitory concentrations (IC 50) and can be statistically assessed based on the area under the neutralization titration curves (AUC). IC 50 values of exemplary anti-IL-20 antibodies of the present invention are described and shown herein, e.g., exemplary antibody 20A10 has a human IL-20 IC 50 value of 329.7 ng/ml in the KZ136-NLuc reporter assay and an IC 50 value of 4.03 ng/ml in the chemiluminescent cell-binding assay (FIG. 5F, left column and FIG. 8D, respectively).

### Peptides and Polypeptides:

The term “peptide” is understood to include the terms “polypeptide” and “protein” (which, at times, may be used interchangeably herein) and any amino acid sequence such as those of the heavy and light chain variable region as well as constant region of the present invention within its meaning. Similarly, fragments of proteins and polypeptides are also contemplated and may be referred to herein as “peptides”. Nevertheless, the term “peptide” preferably denotes an amino acid polymer including at least 5 contiguous amino acids, preferably at least 10 contiguous amino acids, more preferably at least 15 contiguous amino acids, still more preferably at least 20 contiguous amino acids, and particularly preferred at least 25 contiguous amino acids. In addition, the peptide in accordance with present invention typically has no more than 100 contiguous amino acids, preferably less than 80 contiguous amino acids and more preferably less than 50 contiguous amino acids.

As used herein, the term “polypeptide” is intended to encompass a singular “polypeptide” as well as plural “polypeptides” such as antibodies of the present invention, and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “dipeptides,” “tripeptides, “oligopeptides,” “protein,” “amino acid chain,” or any other term used to refer to a chain or chains of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Nevertheless, the term “polypeptide” preferably denotes an amino acid polymer including at least 100 amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides that have been separated, fractionated, or partially or substantially purified by any suitable technique.

“Recombinant peptides, polypeptides or proteins” refer to peptides, polypeptides or proteins produced by recombinant DNA techniques, i.e. produced from cells, microbial or mammalian, transformed by an exogenous recombinant DNA expression construct encoding the fusion protein including the desired peptide. Proteins or peptides expressed in most bacterial cultures will typically be free of glycan. Proteins or polypeptides expressed in yeast may have a glycosylation pattern different from that expressed in mammalian cells.

Also included as polypeptides of the present invention are fragments, derivatives, analogs and variants of the foregoing polypeptides and any combination thereof. The terms “fragment,” “variant,” “derivative” and “analog” include peptides and polypeptides having an amino acid sequence sufficiently similar to the amino acid sequence of the natural peptide. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that comprise a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100%, identical are defined herein as sufficiently similar. Preferably, variants will be sufficiently similar to the amino acid sequence of the preferred peptides of the present invention, in particular to antibodies or antibody fragments, or to synthetic peptide or peptide-based compound comprising epitopes recognized by the antibodies of the present invention or fragments, variants, derivatives or analogs of either of them. Such variants generally retain the functional activity of the peptides of the present invention, i.e. are bound by the antibodies of the present invention. Variants include peptides that differ in amino acid sequence from the native and wt peptide, respectively, by way of one or more amino acid deletion(s), addition(s), and/or substitution(s). These may be naturally occurring variants as well as artificially designed ones.

The terms “fragment,” “variant,” “derivative” and “analog” when referring to antibodies or antibody polypeptides of the present invention include any polypeptides that retain at least some of the antigen-binding properties of the corresponding native binding molecule, antibody, or polypeptide. Fragments of polypeptides of the present invention include proteolytic fragments, as well as deletion fragments, in addition to specific antibody fragments discussed elsewhere herein. Variants of antibodies and antibody polypeptides of the present invention include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of binding molecules of the present invention, e.g., antibodies and antibody polypeptides of the present invention, are polypeptides which have been altered so as to exhibit additional features not found on the native polypeptide. Examples include fusion proteins. Variant polypeptides may also be referred to herein as “polypeptide analogs”. As used herein a “derivative” of a binding molecule or fragment thereof, an antibody, or an antibody polypeptide refers to a subject polypeptide having one or more residues chemically derivatized by reaction of a functional side group. Also included as “derivatives” are peptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

### Anti-Idiotypic Antibodies:

The term “anti-idiotypic antibodies” when referring to antibodies or other binding molecules includes molecules which bind to the unique antigenic peptide sequence located on an antibody's variable region near or at the antigen binding site, inhibiting by this a specific immune response otherwise caused by the given auto-antibody. In an analogous manner synthetic peptide or peptide-based compound comprising an epitope specifically recognized by an antibody of the present invention may be used.

Anti-idiotypic antibodies may be obtained in a similar fashion as other antibodies. The particular anti-idiotypic antibody is detected by any sort of cross-linking, either by agglutination (in turbidimetric or nephelometric assays), precipitation (radial immunodiffusion), or sandwich immunoassays such as ELISAs. U.S. patent application No. 20020142356 provides a method for obtaining anti-idiotypic monoclonal antibody populations directed to an antibody that is specific for a high-concentration, high-molecular-weight target antigen wherein said anti-idiotypic antibody populations have a wide range of binding affinities for the selected antibody specific to said target antigen and wherein a subset of said anti-idiotypic antibody populations can be selected having the required affinity for a particular application.

U.S. patent application No. 20020142356 describes a competitive immunoassay of an antigen using an antibody as coat and an anti-idiotypic antibody as detection or vice-versa. Other references disclosing use of an anti-idiotypic antibody as a surrogate antigen include Losman et al. Cancer Research 55 (1995) (23 suppl. S):S5978-S5982; Becker et al. J. Immunol. Methods 192 (1996), 73-85; Baral et al. Int. J. Cancer 92 (2001), 88-95; and Kohen et al. Food and Agriculture Immunology 12 (2000), 193-201. Use of anti-idiotypic antibodies in treatment of diseases or against parasites is known in the art; see, e.g., in Sacks et al. J. Exp. Medicine 155 (1982), 1108-1119.

Determination of Similarity and/or Identity of Molecules:

“Similarity” between two peptides is determined by comparing the amino acid sequence of one peptide to the sequence of a second peptide. An amino acid of one peptide is similar to the corresponding amino acid of a second peptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos EMBO J. 8 (1989), 779-785. For example, amino acids belonging to one of the following groups represent conservative changes or substitutions: -Ala, Pro, Gly, Gln, Asn, Ser, Thr; -Cys, Ser, Tyr, Thr; -Val, Ile, Leu, Met, Ala, Phe; -Lys, Arg, His; -Phe, Tyr, Trp, His; and -Asp, Glu.

The determination of percent identity or similarity between two sequences is preferably accomplished using the mathematical algorithm of Karlin and Altschul (1993) Proc. Natl. Acad. Sci USA 90: 5873-5877. Such an algorithm is incorporated into the BLASTn and BLASTp programs of Altschul et al. (1990) J. Mol. Biol. 215: 403-410 available at NCBI (http://www.ncbi.nlm.nih.gov/blast/Blast.cge).

The determination of percent identity or similarity is performed with the standard parameters of the BLASTn and BLASTp programs.

BLAST polynucleotide searches are performed with the BLASTn program. For the general parameters, the “Max Target Sequences” box may be set to 100, the “Short queries” box may be ticked, the “Expect threshold” box may be set to 10 and the “Word Size” box may be set to 28. For the scoring parameters the “Match/mismatch Scores” may be set to 1,−2 and the “Gap Costs” box may be set to linear. For the Filters and Masking parameters, the “Low complexity regions” box may not be ticked, the “Species-specific repeats” box may not be ticked, the “Mask for lookup table only” box may be ticked, and the “Mask lower case letters” box may not be ticked.

BLAST protein searches are performed with the BLASTp program. For the general parameters, the “Max Target Sequences” box may be set to 100, the “Short queries” box may be ticked, the “Expect threshold” box may be set to 10 and the “Word Size” box may be set to “3”. For the scoring parameters the “Matrix” box may be set to “BLOSUM62”, the “Gap Costs” Box may be set to “Existence: 11 Extension: 1”, the “Compositional adjustments” box may be set to “Conditional compositional score matrix adjustment”. For the Filters and Masking parameters the “Low complexity regions” box may not be ticked, the “Mask for lookup table only” box may not be ticked and the “Mask lower case letters” box may not be ticked.

### Polynucleotides:

The term “polynucleotide” is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid that consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or not fused to a nucleic acid encoding a binding molecule, an antibody, or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” or “operably linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide of the present invention is RNA, for example, in the form of messenger RNA (mRNA), small hairpin RNA (shRNA), small interfering RNA (siRNA) or any other RNA product.

Polynucleotide and nucleic acid coding regions of the present invention may be associated with additional coding regions which encode secretory or signal peptides, which direct the secretion of a polypeptide encoded by a polynucleotide of the present invention. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the complete or “full-length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase. However, intracellular production of the polypeptides, in particular of the immunoglobulins and fragments thereof of the present invention is also possible.

### Expression:

The term “expression” as used herein refers to a process by which a gene produces a biochemical, for example, an RNA or polypeptide. The process includes any manifestation of the functional presence of the gene within the cell including, without limitation, gene knockdown as well as both transient expression and stable expression. It includes without limitation transcription of the gene into messenger RNA (mRNA), transfer RNA (tRNA), small hairpin RNA (shRNA), small interfering RNA (siRNA) or any other RNA product, and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., small interfering RNA (siRNA), a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

To express the peptide, polypeptide or fusion protein (hereinafter referred to as “product”) in a host cell, a procedure such as the following can be used. A restriction fragment containing a DNA sequence that encodes said product may be cloned into an appropriate recombinant plasmid containing an origin of replication that functions in the host cell and an appropriate selectable marker. The plasmid may include a promoter for inducible expression of the product (e.g., pTrc (Amann et al Gene 69 (1988), 301 315) and pET1 Id (Studier et al. Gene Expression Technology: Methods in Enzymology 185, Academic Press, San Diego, Calif. (1990), 60 89). The recombinant plasmid may be introduced into the host cell by, for example, electroporation and cells containing the recombinant plasmid may be identified by selection for the marker on the plasmid. Expression of the product may be induced and detected in the host cell using an assay specific for the product.

In some embodiments, the DNA that encodes the product/peptide may be optimized for expression in the host cell. For example, the DNA may include codons for one or more amino acids that are predominant in the host cell relative to other codons for the same amino acid.

Alternatively, the expression of the product may be performed by in vitro synthesis of the protein in cell-free extracts which are also particularly suited for the incorporation of modified or unnatural amino acids for functional studies; see also infra. The use of in vitro translation systems can have advantages over in vivo gene expression when the over-expressed product is toxic to the host cell, when the product is insoluble or forms inclusion bodies, or when the protein undergoes rapid proteolytic degradation by intracellular proteases. The most frequently used cell-free translation systems consist of extracts from rabbit reticulocytes, wheat germ and Escherichia coli. All are prepared as crude extracts containing all the macromolecular components (70S or 80S ribosomes, tRNAs, aminoacyl-tRNA synthetases, initiation, elongation and termination factors, etc.) required for translation of exogenous RNA. To ensure efficient translation, each extract must be supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors known in the art (Mg²⁺, K⁺, etc.). Appropriate transcription/translation systems are commercially available, for example from Promega Corporation, Roche Diagnostics, and Ambion, i.e. Applied Biosystems (Anderson et al. Meth. Enzymol. 101 (1983), 635-644; Arduengo et al. The Role of Cell-Free Rabbit Reticulocyte Expression Systems in Functional Proteomics in Kudlicki, Katzen and Bennett eds., Cell-Free Expression Vol. 2007. Austin, Tx: Landes Bioscience, pp. 1-18; Chen and Zubay Meth. Enzymol. 101 (1983), 674-90; Ezure et al. Biotechnol. Prog. 22 (2006), 1570-1577).

### Host Cells:

In respect of the present invention, host cell can be any prokaryotic or eukaryotic cell, such as a bacterial, insect, fungal, plant, animal or human cell. Preferred fungal cells are, for example, those of the genus Saccharomyces, in particular those of the species S. cerevisiae. The term “prokaryotic” is meant to include all bacteria which can be transformed or transfected with a DNA or RNA molecules for the expression of an antibody of the invention or the corresponding immunoglobulin chains. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells, most preferably HEK 293, NSO, CSO and CHO cells. Depending upon the host employed in a recombinant production procedure, the antibodies or immunoglobulin chains encoded by the polynucleotide of the present invention may be glycosylated or may be non-glycosylated. Antibodies of the invention or the corresponding immunoglobulin chains may also include an initial methionine amino acid residue. A polynucleotide of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the antibody of the invention or the corresponding immunoglobulin chains in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells. Suitable source cells for the DNA sequences and host cells for immunoglobulin expression and secretion can be obtained from a number of sources, such as the American Type Culture Collection (“Catalogue of Cell Lines and Hybridomas,” Fifth edition (1985) Rockville, Md., U.S.A., which is incorporated herein by reference). Furthermore, transgenic animals, preferably mammals, comprising cells of the invention may be used for the large scale production of the antibody of the invention.

The transformed hosts can be grown in fermentors and cultured according to techniques known in the art to achieve optimal cell growth. Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis and the like; see, Scopes, “Protein Purification”, Springer Verlag, N.Y. (1982). The antibody or its corresponding immunoglobulin chain(s) of the invention can then be isolated from the growth medium, cellular lysates, or cellular membrane fractions. The isolation and purification of the, e.g., recombinantly expressed antibodies or immunoglobulin chains of the invention may be by any conventional means such as, for example, preparative chromatographic separations and immunological separations such as those involving the use of monoclonal or polyclonal antibodies directed, e.g., against the constant region of the antibody of the invention. It will be apparent to those skilled in the art that the antibodies of the invention can be further coupled to other moieties for, e.g., drug targeting and imaging applications. Such coupling may be conducted chemically after expression of the antibody or antigen to site of attachment or the coupling product may be engineered into the antibody or antigen of the invention at the DNA level. The DNAs are then expressed in a suitable host system, and the expressed proteins are collected and renatured, if necessary.

Substantially pure immunoglobulins of at least about 90 to 95% homogeneity are preferred, and 98 to 99% or more homogeneity most preferred, for pharmaceutical uses. Once purified, partially or to homogeneity as desired, the antibodies may then be used therapeutically (including extracorporally) or in developing and performing assay procedures.

The present invention also involves a method for producing cells capable of expressing an antibody of the invention or its corresponding immunoglobulin chain(s) comprising genetically engineering cells with the polynucleotide or with the vector of the invention. The cells obtainable by the method of the invention can be used, for example, to test the interaction of the antibody of the invention with its antigen.

### ELISA-Assays:

Enzyme-linked immunosorbent assays (ELISAs) for various antigens include those based on colorimetry, chemiluminescence, and fluorometry. ELISAs have been successfully applied in the determination of low amounts of drugs and other antigenic components in plasma and urine samples, involve no extraction steps, and are simple to carry out. ELISAs for the detection of antibodies to protein antigens often use direct binding of short synthetic peptides to the plastic surface of a microtitre plate. The peptides are, in general, very pure due to their synthetic nature and efficient purification methods using high-performance liquid chromatography. A drawback of short peptides is that they usually represent linear, but not conformational or discontinuous epitopes. To present conformational epitopes, either long peptides or the complete native protein is used. Direct binding of the protein antigens to the hydrophobic polystyrene support of the plate can result in partial or total denaturation of the bound protein and loss of conformational epitopes. Coating the plate with an antibody, which mediates the immobilization (capture ELISA) of the antigens, can avoid this effect.

However, frequently, overexpressed recombinant proteins are insoluble and require purification under denaturing conditions and renaturation, when antibodies to conformational epitopes are to be analyzed. See, for example, U.S. patent application No. 20030044870 for a generic ELISA using recombinant fusion proteins as coat proteins.

### Binding Molecules:

A “binding molecule” as used in the context of the present invention relates primarily to antibodies, and fragments thereof, but may also refer to other non-antibody molecules that bind to the “molecules of interest” of the present invention, wherein the molecules of interest are proteins of the class of glycoproteins known as cytokines, in particular IL-20. The molecules of interest of the present invention are defined in further detail within the description of the particular embodiments of the present invention above and below. The binding molecules of the present invention include but are not limited to hormones, receptors, ligands, major histocompatibility complex (MHC) molecules, chaperones such as heat shock proteins (HSPs) as well as cell-cell adhesion molecules such as members of the cadherin, intergrin, C-type lectin and immunoglobulin (Ig) superfamilies. Thus, for the sake of clarity only and without restricting the scope of the present invention most of the following embodiments are discussed with respect to antibodies and antibody-like molecules which represent the preferred binding molecules for the development of therapeutic and diagnostic agents.

### Antibodies:

The terms “antibody” and “immunoglobulin” are used interchangeably herein. An antibody or immunoglobulin is a molecule binding to a molecule of interest of the present invention as defined hereinabove and below, which comprises at least the variable domain of a heavy chain, and normally comprises at least the variable domains of a heavy chain and a light chain. Basic immunoglobulin structures in vertebrate systems are relatively well understood; see, e.g., Harlow and Lane, Antibodies: A Laboratory Manual, (Cold Spring Harbor Laboratory Press, 2nd ed. 1988). The terms “binds” and “recognizes” are used interchangeably in respect of the binding affinity of the binding molecules of the present invention, e.g., antibodies.

Any antibody or immunoglobulin fragment which contains sufficient structure to specifically bind to the molecules of interest, as defined hereinabove and below, is denoted herein interchangeably as a “binding molecule”, “binding fragment” or an “immunospecific fragment.”

Antibodies or antigen-binding fragments, immunospecific fragments, variants, or derivatives thereof of the invention include, but are not limited to, polyclonal, monoclonal, multispecific, human, humanized, primatized, murinized or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a V_{L} or V_{H} domain, fragments produced by a Fab expression library, and anti-idiotypic (anti-Id) antibodies (including, e.g., anti-Id antibodies to antibodies disclosed herein). ScFv molecules are known in the art and are described, e.g., in U.S. Pat. No. 5,892,019. In this respect, antigen-binding fragment of the antibody can be as well domain antibodies (dAb) also known as single domain antibodies (sdAB) or Nanobodies™ (Ablynx, Gent, Belgium), see, e.g., De Haard et al. J. Bacteriol. 187 (2005), 4531-4541; Holt et al. Trends Biotechnol. 21 (2003), 484-490. As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgE, IgM, IgD, IgA, and IgY, respectively. The immunoglobulin subclasses (isotypes) e.g., IgG1, IgG2, IgG3, IgG4, IgA1, etc. are well characterized and are known to confer functional specialization. Immunoglobulin or antibody molecules of the invention can be of any type (e.g., IgG, IgE, IgM, IgD, IgA, and IgY), class (e.g., IgG1, IgG2, IgG3, IgG4, IgA1, IgA2, etc.) or subclass of immunoglobulin molecule. Modified versions of each of these classes and isotypes are readily discernable to the skilled artisan in view of the instant disclosure and, accordingly, are within the scope of the instant invention. Although all immunoglobulin classes are clearly within the scope of the present invention, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

As evident from the classification of the exemplary anti-IL-20 antibodies of the present invention enlisted in Table 1 above, the exemplary antibodies of the present invention are of the IgG4 or IgG1 class, possibly implicating regulatory T-cell responses and/or epithelia in their initiation in these AIRE-deficiency states. These findings are confirmed by the classification of corresponding autoantibodies found in the AIRE-deficient mice described by Kärner et al. in Clin. Exp. Immunol. (2012); doi: 10.1111/cei.12024, the disclosure content of which is incorporated herein by reference. Accordingly, in a preferred embodiment of the present invention, the antibodies of the present invention are of the IgG type, even more preferred IgG4 or IgG1.

### IgG Structure:

Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CL domains actually comprise the carboxy-terminus of the heavy and light chain, respectively.

As indicated above, the variable region allows the antibody to selectively recognize and specifically bind epitopes on antigens. That is, the V_{L} domain and V_{H} domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the V_{H} and V_{L} chains. Any antibody or immunoglobulin fragment which contains sufficient structure to specifically bind to a molecule of interest of the present invention is denoted herein interchangeably as a “binding fragment” or an “immunospecific fragment.”

In naturally occurring antibodies, an antibody comprises six hypervariable regions, sometimes called “complementarity determining regions” or “CDRs” present in each antigen-binding domain, which are short, non-contiguous sequences of amino acids that are specifically positioned to form the antigen-binding domain as the antibody assumes its three dimensional configuration in an aqueous environment. The “CDRs” are flanked by four relatively conserved “framework” regions or “FRs” which show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined; see, “Sequences of Proteins of Immunological Interest,” Kabat et al. U.S. Department of Health and Human Services, (1983); and Chothia and Lesk J. Mol. Biol. 196 (1987), 901-917, which are incorporated herein by reference in their entireties.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al. U.S. Dept. of Health and Human Services, “Sequences of Proteins of Immunological Interest” (1983) and by Chothia and Lesk J. Mol. Biol. 196 (1987), 901-917, which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 2 as a comparison. The exact residue numbers which encompass a particular CDR will vary depending on the sequence and size of the CDR. Those skilled in the art can routinely determine which residues comprise a particular hypervariable region or CDR of the human IgG subtype of antibody given the variable region amino acid sequence of the antibody.

TABLE 2
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 2 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. U.S. Dept. of Health and Human Services, “Sequence of Proteins of Immunological Interest” (1983). Unless otherwise specified, references to the numbering of specific amino acid residue positions in an antibody or antigen-binding fragment, variant, or derivative thereof of the present invention are according to the Kabat numbering system, which however is theoretical and may not equally apply to every antibody of the present invention. For example, depending on the position of the first CDR the following CDRs might be shifted in either direction.

In one embodiment, the antibody of the present invention is not IgM or a derivative thereof with a pentavalent structure. Particular, in specific applications of the present invention, especially therapeutic use, IgMs are less useful than IgG and other bivalent antibodies or corresponding binding molecules since IgMs due to their pentavalent structure and lack of affinity maturation often show unspecific cross-reactivities and very low affinity.

In a particularly preferred embodiment, the antibody of the present invention is not a polyclonal antibody, i.e. it substantially consists of one particular antibody species rather than being a mixture obtained from a plasma immunoglobulin sample.

### Antibody Fragments, Animalization:

Antibody fragments, including single-chain antibodies, may comprise the variable region(s) alone or in combination with the entirety or a portion of the following: hinge region, CH1, CH2, and CH3 domains. Also included in the invention are fragments binding to a molecule of interest of the present invention, said fragments comprising any combination of variable region(s) with a hinge region, CH1, CH2, and CH3 domains. Antibodies or immunospecific fragments thereof of the present invention equivalent to the monoclonal antibodies isolated in accordance with the method of the present invention, in particular to the human monoclonal antibodies may be from any animal origin including birds and mammals. Preferably, the antibodies are human, murine, donkey, rabbit, goat, guinea pig, camel, llama, horse, or chicken antibodies. In another embodiment, the variable region may be chondrichthoid in origin (e.g., from sharks).

In a particularly preferred embodiment of the present invention, the antibodies are naturally occurring human monoclonal antibodies or binding fragments, derivatives and variants thereof cloned from human subjects, which bind specifically to IL-20 of the present invention, as defined in detail above and below, e.g., in Table 1, the Figures, in particular FIGS. 3 to 10 and in the Examples, e.g., in Examples 2 and 5.

Optionally, the framework region of the human antibody is aligned and adopted in accordance with the pertinent human germ line variable region sequences in the database; see, e.g., Vbase (http://vbase.mrc-cpe.cam.ac.uk/) hosted by the MRC Centre for Protein Engineering (Cambridge, UK). For example, amino acids considered to potentially deviate from the true germ line sequence could be due to the PCR primer sequences incorporated during the cloning process. Compared to artificially generated human-like antibodies such as single chain antibody fragments (scFvs) from a phage displayed antibody library or xenogeneic mice the human monoclonal antibody of the present invention is characterized by (i) being obtained using the human immune response rather than that of animal surrogates, i.e. the antibody has been generated in response to natural IL-20 in its relevant conformation in the human body, (ii) having protected the individual from or at least significantly minimized the presence of symptoms of a disease, e.g., SLE, and (iii) since the antibody is of human origin the risks of cross-reactivity against self-antigens is minimized. Thus, in accordance with the present invention the terms “human monoclonal antibody”, “human monoclonal autoantibody”, “human antibody” and the like are used to denote an IL-20 binding molecule which is of human origin, i.e. which has been isolated from a human cell such as a B cell or hybridoma thereof or the cDNA of which has been directly cloned from mRNA of a human cell, for example a human memory B cell. A human antibody is still considered as “human” even if amino acid substitutions are made in the antibody, e.g., to improve its binding characteristics.

Antibodies derived from human immunoglobulin libraries or from animals transgenic for one or more human immunoglobulins and that do not express endogenous immunoglobulins, as described infra and, for example, in U.S. Pat. No. 5,939,598 by Kucherlapati et al. are denoted human-like antibodies in order to distinguish them from truly human antibodies of the present invention.

For example, the paring of heavy and light chains of human-like antibodies such as synthetic and semi-synthetic antibodies typically isolated from phage display do not necessarily reflect the original paring as it occurred in the original human B cell. Accordingly Fab and scFv fragments obtained from recombinant expression libraries as commonly used in the prior art can be considered as being artificial with all possible associated effects on immunogenicity and stability.

In contrast, the present invention provides isolated affinity-matured antibodies from selected human subjects, which are characterized by their therapeutic utility.

### Grafted Antibodies (Equivalents)

The invention also relates to grafted antibodies (interchangeably referred to as equivalents) containing CDRs derived from the antibodies of the present invention, such as IL-20 antibodies, respectively. Such grafted CDRs include animalized antibodies, in which CDRs from the antibodies of the present invention have been grafted or in which a CDR containing one or more amino acid substitutions is grafted. The CDRs can be grafted directly into a human framework or an antibody framework from animal origin as indicated above. If desired, framework changes can also be incorporated by generating framework libraries. The optimization of CDRs and/or framework sequences can be performed independently and sequentially combined or can be performed simultaneously, as described in more detail below.

To generate grafted antibodies donor CDRs of the antibodies of the present invention are grafted onto an antibody acceptor variable region framework. Methods for grafting antibodies and generating CDR variants to optimize activity have been described previously (see, e.g., international patent applications WO98/33919; WO00/78815; WO01/27160). The procedure can be performed to achieve grafting of donor CDRs and affinity reacquisition in a simultaneous process. The methods similarly can be used, either alone or in combination with CDR grafting, to modify or optimize the binding affinity of a variable region. The methods for conferring donor CDR binding affinity onto an acceptor variable region are applicable to both heavy and light chain variable regions and as such can be used to simultaneously graft and optimize the binding affinity of an antibody variable region.

The donor CDRs can be altered to contain a plurality of different amino acid residue changes at all or selected positions within the donor CDRs. For example, random or biased incorporation of the twenty naturally occurring amino acid residues, or preselected subsets, can be introduced into the donor CDRs to produce a diverse population of CDR species. Inclusion of CDR variant species into the diverse population of variable regions allows for the generation of variant species that exhibit optimized binding affinity for a predetermined antigen. A range of possible changes can be made in the donor CDR positions. Some or all of the possible changes that can be selected for can be introduced into the population of grafted donor CDRs. A single position in a CDR can be selected to introduce changes or a variety of positions having altered amino acids can be combined and screened for activity.

One approach is to change all amino acid positions along a CDR by replacement at each position with, for example, all twenty naturally occurring amino acids. The replacement of each position can occur in the context of other donor CDR amino acid positions so that a significant portion of the CDR maintains the authentic donor CDR sequence, and therefore, the binding affinity of the donor CDR. For example, an acceptor variable region framework, either a native or altered framework, can be grafted with a population of CDRs containing single position replacements at each position within the CDRs. Similarly, an acceptor variable region framework can be targeted for grafting with a population of CDRs containing more than one position changed to incorporate all twenty amino acid residues, or a subset of amino acids. One or more amino acid positions within a CDR, or within a group of CDRs to be grafted, can be altered and grafted into an acceptor variable region framework to generate a population of grafted antibodies. It is understood that a CDR having one or more altered positions can be combined with one or more other CDRs having one or more altered positions, if desired.

A population of CDR variant species having one or more altered positions can be combined with any or all of the CDRs which constitute the binding pocket of a variable region. Therefore, an acceptor variable region framework can be targeted for the simultaneous incorporation of donor CDR variant populations at one, two or all three recipient CDR locations in a heavy or light chain. The choice of which CDR or the number of CDRs to target with amino acid position changes will depend on, for example, if a full CDR grafting into an acceptor is desired or whether the method is being performed for optimization of binding affinity.

Another approach for selecting donor CDR amino acids to change for conferring donor CDR binding affinity onto an antibody acceptor variable region framework is to select known or readily identifiable CDR positions that are highly variable. For example, the variable region CDR3 is generally highly variable. This region therefore can be selectively targeted for amino acid position changes during grafting procedures to ensure binding affinity reacquisition or augmentation, either alone or together with relevant acceptor variable framework changes.

### Murinized Antibodies:

An example of antibodies generated by grafting, as described above, are murinized antibodies. As used herein, the term “murinized antibody” or “murinized immunoglobulin” refers to an antibody comprising one or more CDRs from a human antibody of the present invention; and a human framework region that contains amino acid substitutions and/or deletions and/or insertions that are based on a mouse antibody sequence. The human immunoglobulin providing the CDRs is called the “parent” or “acceptor” and the mouse antibody providing the framework changes is called the “donor”. Constant regions need not be present, but if they are, they are usually substantially identical to mouse antibody constant regions, i.e. at least about 85-90%, preferably about 95% or more identical. Hence, in some embodiments, a full-length murinized human heavy or light chain immunoglobulin contains a mouse constant region, human CDRs, and a substantially human framework that has a number of “murinizing” amino acid substitutions. Typically, a “murinized antibody” is an antibody comprising a murinized variable light chain and/or a murinized variable heavy chain. For example, a murinized antibody would not encompass a typical chimeric antibody, e.g., because the entire variable region of a chimeric antibody is non-mouse. A modified antibody that has been “murinized” by the process of “murinization” binds to the same antigen as the parent antibody that provides the CDRs and is usually less immunogenic in mice, as compared to the parent antibody.

### Antibody Fragments:

As used herein, the term “heavy chain portion” includes amino acid sequences derived from an immunoglobulin heavy chain. A polypeptide comprising a heavy chain portion comprises at least one of: a CH1 domain, a hinge (e.g., upper, middle, and/or lower hinge region) domain, a CH2 domain, a CH3 domain, or a variant or fragment thereof. For example, a binding polypeptide for use in the invention may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the invention comprises a polypeptide chain comprising a CH3 domain. Further, a binding polypeptide for use in the invention may lack at least a portion of a CH2 domain (e.g., all or part of a CH2 domain). As set forth above, it will be understood by one of ordinary skill in the art that these domains (e.g., the heavy chain portions) may be modified such that they vary in amino acid sequence from the naturally occurring immunoglobulin molecule.

In certain antibodies, or antigen-binding fragments, variants, or derivatives thereof disclosed herein, the heavy chain portions of one polypeptide chain of a multimere are identical to those on a second polypeptide chain of the multimere. Alternatively, heavy chain portion-containing monomers of the invention are not identical. For example, each monomer may comprise a different target binding site, forming, for example, a bispecific antibody or diabody.

In another embodiment, the antibodies, or antigen-binding fragments, variants, or derivatives thereof disclosed herein are composed of a single polypeptide chain such as scFvs and are to be expressed intracellularly (intrabodies) for potential in vivo therapeutic and diagnostic applications.

The heavy chain portions of a binding polypeptide for use in the diagnostic and treatment methods disclosed herein may be derived from different immunoglobulin molecules. For example, a heavy chain portion of a polypeptide may comprise a CH1 domain derived from an IgG1 molecule and a hinge region derived from an IgG3 molecule. In another example, a heavy chain portion can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

Thus, as also exemplified in the Examples, in one embodiment the constant region of the antibody of the present invention or part thereof, in particular the CH2 and/or CH3 domain but optionally also the CH1 domain is heterologous to the variable region of the native human monoclonal antibody isolated in accordance with the method of the present invention. In this context, the heterologous constant region(s) are preferably of human origin in case of therapeutic applications of the antibody of the present invention but could also be of for example rodent origin in case of animal studies; see also the Examples.

As used herein, the term “light chain portion” includes amino acid sequences derived from an immunoglobulin light chain. Preferably, the light chain portion comprises at least one of a V_{L} or CL domain.

As previously indicated, the subunit structures and three-dimensional configuration of the constant regions of the various immunoglobulin classes are well known. As used herein, the term “V_{H} domain” includes the amino terminal variable domain of an immunoglobulin heavy chain and the term “CH1 domain” includes the first (most amino terminal) constant region domain of an immunoglobulin heavy chain. The CH1 domain is adjacent to the V_{H} domain and is amino terminal to the hinge region of an immunoglobulin heavy chain molecule.

As used herein the term “CH2 domain” includes the portion of a heavy chain molecule that extends, e.g., from about residue 244 to residue 360 of an antibody using conventional numbering schemes (residues 244 to 360, Kabat numbering system; and residues 231-340, EU numbering system; see Kabat et al, supra). The CH2 domain is unique in that it is not closely paired with another domain. Rather, two N-linked branched carbohydrate chains are interposed between the two CH2 domains of an intact native IgG molecule. It is also well documented that the CH3 domain extends from the CH2 domain to the C-terminal region of the IgG molecule and comprises approximately 108 residues.

As used herein, the term “hinge region” includes the portion of a heavy chain molecule that joins the CH1 domain to the CH2 domain. This hinge region comprises approximately 25 residues and is flexible, thus allowing the two N-terminal antigen-binding regions to move independently. Hinge regions can be subdivided into three distinct domains: upper, middle, and lower hinge domains; see Roux et al. J. Immunol. 161 (1998), 4083.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the terms “linked”, “fused” or “fusion” are used interchangeably. These terms refer to the joining together of two or more elements or components, by whatever means including chemical conjugation or recombinant means. An “in-frame fusion” refers to the joining of two or more polynucleotide open reading frames (ORFs) to form a continuous longer ORF, in a manner that maintains the correct translational reading frame of the original ORFs. Thus, a recombinant fusion protein is a single protein containing two or more segments that correspond to polypeptides encoded by the original ORFs (which segments are not normally so joined in nature). Although the reading frame is thus made continuous throughout the fused segments, the segments may be physically or spatially separated by, for example, an in-frame linker sequence. For example, polynucleotides encoding the CDRs of an immunoglobulin variable region may be fused, in-frame, but be separated by a polynucleotide encoding at least one immunoglobulin framework region or additional CDR regions, as long as the “fused” CDRs are co-translated as part of a continuous polypeptide.

### Epitopes:

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, a peptide or polypeptide epitope recognized by antibodies of the present invention contains a sequence of at least 4, at least 5, at least 6, at least 7, more preferably at least 8, at least 9, at least 10, at least 15, at least 20, at least 25, between about 15 to about 30 or between about 30 to about 50 contiguous or non-contiguous amino acids of a molecule of interest of the present invention, i.e. IL-20. Preferably, the peptide recognized by an antibody of the present invention contains 15 to 18 amino acids. In a preferred embodiment, the peptide contains 18 amino acids consisting of a contiguous sequence from human IL-20 with the sequence PDHYTLRKISSLANSFLT SEQ ID NO: 69. In another preferred embodiment, the peptide epitope containing 15 amino acids consisting of the sequence DHYTLRKISSLANSF SEQ ID NO: 70.

### Binding Characteristics:

By “binding” or “recognizing”, used interchangeably herein, it is generally meant that a binding molecule, e.g., an antibody binds to a predetermined epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D”. Unrelated epitopes are usually part of a nonspecific antigen (e.g., BSA, casein, or any other specified polypeptide), which may be used for the estimation of the binding specificity of a given binding molecule. In this respect, term “specific binding” refers to antibody binding to a predetermined antigen with a K_{D} that is at least twofold less than its K_{D} for binding to a nonspecific antigen. The term “highly specific” binding as used herein means that the relative K_{D} of the antibody for the specific target epitope is at least 10-fold less than the K_{D} for binding that antibody to other ligands.

Where present, the term “immunological binding characteristics,” or other binding characteristics of an antibody with an antigen, in all of its grammatical forms, refers to the specificity, affinity, cross-reactivity, and other binding characteristics of an antibody.

By “preferentially binding”, it is meant that the binding molecule, e.g., antibody specifically binds to an epitope more readily than it would bind to a related, similar, homologous, or analogous epitope. Thus, an antibody which “preferentially binds” to a given epitope would more likely bind to that epitope than to a related epitope, even though such an antibody may cross-react with the related epitope.

By way of non-limiting example, a binding molecule, e.g., an antibody may be considered to bind a first epitope preferentially if it binds said first epitope with dissociation constant (K_{D}) that is less than the antibody's K_{D} for the second epitope. In another non-limiting example, an antibody may be considered to bind a first antigen preferentially if it binds the first epitope with an affinity that is at least one order of magnitude less than the antibody's K_{D} for the second epitope. In another non-limiting example, an antibody may be considered to bind a first epitope preferentially if it binds the first epitope with an affinity that is at least two orders of magnitude less than the antibody's K_{D} for the second epitope.

In another non-limiting example, a binding molecule, e.g., an antibody may be considered to bind a first epitope preferentially if it binds the first epitope with an off rate (k(off)) that is less than the antibody's k(off) for the second epitope. In another non-limiting example, an antibody may be considered to bind a first epitope preferentially if it binds the first epitope with an affinity that is at least one order of magnitude less than the antibody's k(off) for the second epitope. In another non-limiting example, an antibody may be considered to bind a first epitope preferentially if it binds the first epitope with an affinity that is at least two orders of magnitude less than the antibody's k(off) for the second epitope.

A binding molecule, e.g., an antibody or antigen-binding fragment, variant, or derivative disclosed herein may be said to bind a molecule of interest of the present invention, a fragment or variant thereof with an off rate (k(off)) of less than or equal to 5×10⁻² sec⁻¹, 10⁻² sec⁻¹, 5×10⁻³ sec⁻¹ or 10⁻³ sec⁻¹. More preferably, an antibody of the invention may be said to bind a molecule of interest of the present invention or a fragment or variant thereof with an off rate (k(off)) less than or equal to 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein may be said to bind a molecule of interest of the present invention or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. More preferably, an antibody of the invention may be said to bind a molecule of interest of the present invention or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody is said to competitively inhibit binding of a reference antibody to a given epitope if it preferentially binds to that epitope to the extent that it blocks, to some degree, binding of the reference antibody to the epitope. Competitive inhibition may be determined by any method known in the art, for example, competition ELISA assays. An antibody may be said to competitively inhibit binding of the reference antibody to a given epitope by at least 90%, at least 80%, at least 70%, at least 60%, or at least 50%.

As used herein, the term “affinity” refers to a measure of the strength of the binding of an individual epitope with the CDR of a binding molecule, e.g., an immunoglobulin molecule; see, e.g., Harlow et al. Antibodies: A Laboratory Manual, Cold Spring Harbor Laboratory Press, 2nd ed. (1988) at pages 27-28. As used herein, the term “avidity” refers to the overall stability of the complex between a population of immunoglobulins and an antigen, that is, the functional combining strength of an immunoglobulin mixture with the antigen; see, e.g., Harlow at pages 29-34. Avidity is related to both the affinity of individual immunoglobulin molecules in the population with specific epitopes, and also the valency of the immunoglobulins and the antigen. For example, the interaction between a bivalent monoclonal antibody and an antigen with a highly repeating epitope structure, such as a polymer, would be one of high avidity. The affinity or avidity of an antibody for an antigen can be determined experimentally using any suitable method; see, for example, Berzofsky et al. “Antibody-Antigen Interactions” In Fundamental Immunology, Paul, W. E., Ed., Raven Press New York, N Y (1984), Kuby Janis Immunology, W. H. Freeman and Company New York, N Y (1992), and methods described therein. General techniques for measuring the affinity of an antibody for an antigen include ELISA, RIA, and surface plasmon resonance. The measured affinity of a particular antibody-antigen interaction can vary if measured under different conditions, e.g., salt concentration, pH. Thus, measurements of affinity and other antigen-binding parameters, e.g., K_{D}, IC₅₀, are preferably made with standardized solutions of antibody and antigen, and a standardized buffer.

Binding molecules, e.g., antibodies or antigen-binding fragments, variants or derivatives thereof of the invention may also be described or specified in terms of their cross-reactivity. As used herein, the term “cross-reactivity” refers to the ability of an antibody, specific for one antigen, to react with a second antigen; a measure of relatedness between two different antigenic substances. Thus, an antibody is cross reactive if it binds to an epitope other than the one that induced its formation. The cross reactive epitope generally contains many of the same complementary structural features as the inducing epitope, and in some cases, may actually fit better than the original.

For example, certain antibodies have some degree of cross-reactivity, in that they bind related, but non-identical epitopes, e.g., epitopes with at least 95%, at least 90%, at least 85%, at least 80%, at least 75%, at least 70%, at least 65%, at least 60%, at least 55%, and at least 50% identity (as calculated using methods known in the art and described herein) to a reference epitope. An antibody may be said to have little or no cross-reactivity if it does not bind epitopes with less than 95%, less than 90%, less than 85%, less than 80%, less than 75%, less than 70%, less than 65%, less than 60%, less than 55%, and less than 50% identity (as calculated using methods known in the art and described herein) to a reference epitope. An antibody may be deemed “highly specific” for a certain epitope, if it does not bind any other analog, ortholog, or homolog of that epitope.

Binding molecules, e.g., antibodies or antigen-binding fragments, variants or derivatives thereof of the invention may also be described or specified in terms of their binding affinity to a molecule of interest of the present invention. Preferred binding affinities include those with a dissociation constant or K_{D} less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. Typically, the antibody binds with a dissociation constant (K_{D}) of 10⁻⁷ M or less to its predetermined antigen. Preferably, the antibody binds its cognate antigen with a dissociation constant (K_{D}) of 10⁻⁹ M or less and still more preferably with a dissociation constant (K_{D}) of 10⁻¹¹ M or less.

### Modifications of Antibodies:

The immunoglobulin or its encoding cDNAs may be further modified. Thus, in a further embodiment the method of the present invention comprises any one of the step(s) of producing a chimeric antibody, humanized antibody, single-chain antibody, Fab-fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to the same epitope as that of any one of the antibodies provided by the present invention (Schier Human Antibodies Hybridomas 7 (1996), 97-105; Malmborg J. Immunol. Methods 183 (1995), 7-13). The production of chimeric antibodies is described, for example, in international application WO89/09622. Methods for the production of humanized antibodies are described in, e.g., European application EP 239 400 A1 and international application WO90/07861. Further sources of antibodies to be utilized in accordance with the present invention are so-called xenogeneic antibodies. The general principle for the production of xenogeneic antibodies such as human antibodies in mice is described in, e.g., international applications WO91/10741, WO94/02602, WO96/34096 and WO 96/33735. As discussed above, the antibody of the invention may exist in a variety of forms besides complete antibodies; including, for example, Fv, Fab and F(ab)₂, as well as in single chains; see e.g. international application WO88/09344.

The antibodies of the present invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Modifications of the antibody of the invention include chemical and/or enzymatic derivatizations at one or more constituent amino acids, including side chain modifications, backbone modifications, and N- and C-terminal modifications including acetylation, hydroxylation, methylation, amidation, and the attachment of carbohydrate or lipid moieties, cofactors, and the like. Likewise, the present invention encompasses the production of chimeric proteins which comprise the described antibody or some fragment thereof at the amino terminus fused to a heterologous molecule such as a label or a drug. Antigen binding molecules generated this way may be used for drug localization to cells expressing the appropriate surface structures of the diseased cell and tissue, respectively. This targeting and binding to cells could be useful for the delivery of therapeutically or diagnostically active agents and gene therapy/gene delivery. Molecules/particles with an antibody of the invention would bind specifically to cells/tissues expressing the particular antigen of interest, and therefore could have diagnostic and therapeutic use.

### Samples:

As used herein, the term “sample” or “biological sample” refers to any biological material obtained from a subject or patient. In one aspect, a sample can comprise blood, cerebrospinal fluid (“CSF”), or urine. In other aspects, a sample can comprise whole blood, plasma, mononuclear cells enriched from peripheral blood (PBMC) such as lymphocytes (i.e. T-cells, NK-cells or B-cells), monocytes, macrophages, dendritic cells and basophils; and cultured cells (e.g., B-cells from a subject). A sample can also include a biopsy or tissue sample including tumor tissue. In still other aspects, a sample can comprise whole cells and/or a lysate of the cells. In one embodiment a sample comprises peripheral blood mononuclear cells (PBMC). Samples can be collected by methods known in the art.

### Diseases and Disorders:

Unless stated otherwise, the terms “disorder” and “disease” are used interchangeably herein. The term “autoimmune disorder” as used herein is a disease or disorder arising from and directed against an individual's own tissues or organs or a co-segregate or manifestation thereof or resulting condition therefrom. Autoimmune diseases are primarily caused by dysregulation of adaptive immune responses and autoantibodies or autoreactive T cells against self-structures are formed. Nearly all autoimmune diseases have an inflammatory component, too. Autoinflammatory diseases are primarily inflammatory, and some classic autoinflammatory diseases are caused by genetic defects in innate inflammatory pathways. In autoinflammatory diseases, no autoreactive T cells or autoantibodies are found. In many of these autoimmune and autoinflammatory disorders, a number of clinical and laboratory markers may exist, including, but not limited to, hypergammaglobulinemia, high levels of autoantibodies, antigen-antibody complex deposits in tissues, benefit from corticosteroid or immunosuppressive treatments, and lymphoid cell aggregates in affected tissues. Without being limited to a theory regarding B cell mediated autoimmune disorder, it is believed that B cells demonstrate a pathogenic effect in human autoimmune diseases through a multitude of mechanistic pathways, including autoantibody production, immune complex formation, dendritic and T cell activation, cytokine synthesis, direct chemokine release, and providing a nidus for ectopic neo-lymphogenesis. Each of these pathways may participate to different degrees in the pathology of autoimmune diseases.

As used herein, an “autoimmune disorder” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease that can affect multiple organ systems including but not limited to systemic lupus erythematosus (SLE), rheumatoid arthritis (RA), polymyositis, autoimmune polyendocrinopathy syndrome type 1 (APS1)/autoimmune polyendocrinopathy-candidiasis-ectodermal dystrophy (APECED), etc. Preferred such diseases include but are not limited to multiple sclerosis (MS), inflammatory bowel disease (IBD), various forms of autoimmune rheumatologic disorders including but not limited to rheumatoid arthritis, juvenile rheumatoid arthritis, psoriasis, psoriatic arthritis, ankylosing spondylitis, spondyloarthritis, psoriatic arthritis, Sjogren's syndrome, scleroderma, lupus, including but not limited to SLE and lupus nephritis, polymyositis/dermatomyositis, and psoriatic arthritis), autoimmune dermatologic disorders (including but not limited to psoriasis, pemphigus group diseases, bullous pemphigoid diseases, and cutaneous lupus erythematosus), and autoimmune endocrine disorders (including but not limited to diabetic-related autoimmune diseases such as type 1 or insulin dependent diabetes mellitus (T1DM or IDDM), autoimmune thyroid disease (including but not limited to Graves' disease and thyroiditis)) and diseases affecting the generation of autoimmunity including but not limited to autoimmune polyendocrinopathy syndrome type 1 (APS1)/autoimmune polyendocrinopathy-candidiasis-ectodermal dystrophy (APECED) and myasthenia gravis (MG/Thymoma). Preferred diseases include, for example, SLE, RA, spondyloarthritis, psoriatic arthritis, psoriasis, Sjogren's syndrome, Graves' disease, thyroiditis, glomerulonephritis and APS1. Still more preferred are RA and SLE, and most preferred SLE.

Further medical uses of IL-20 antagonists such as anti-IL-20 antibodies have been recently described, for example for preventing or treating rheumatoid arthritis and osteoporosis in international application WO2010/042634; for reducing liver fibrosis in international application WO2014/025767; for the treatment of an allergic airway disorder (e.g., asthma or bronchial airway obstruction) international application WO2014/025775 and for promoting bone fracture healing in a subject having a bone fracture in international application WO2014/036384.

### Labels and Diagnostics:

Labeling agents can be coupled either directly or indirectly to the antibodies or antigens of the invention, for example for preparing immuoconjugates. One example of indirect coupling is by use of a spacer moiety. Furthermore, the antibodies of the present invention can comprise a further domain, said domain being linked by covalent or non-covalent bonds. The linkage can be based on genetic fusion according to the methods known in the art and described above or can be performed by, e.g., chemical cross-linking as described in, e.g., international application WO94/04686. The additional domain present in the fusion protein comprising the antibody of the invention may preferably be linked by a flexible linker, advantageously a polypeptide linker, wherein said polypeptide linker comprises plural, hydrophilic, peptide-bonded amino acids of a length sufficient to span the distance between the C-terminal end of said further domain and the N-terminal end of the antibody of the invention or vice versa. The therapeutically or diagnostically active agent can be coupled to the antibody of the invention or an antigen-binding fragment thereof by various means. This includes, for example, single-chain fusion proteins comprising the variable regions of the antibody of the invention coupled by covalent methods, such as peptide linkages, to the therapeutically or diagnostically active agent. Further examples include molecules which comprise at least an antigen-binding fragment coupled to additional molecules covalently or non-covalently including those in the following non-limiting illustrative list. Traunecker, Int. J. Cancer Surp. SuDP 7 (1992), 51-52, describe the bispecific reagent janusin in which the Fv region directed to CD3 is coupled to soluble CD4 or to other ligands such as OVCA and IL-7. Similarly, the variable regions of the antibody of the invention can be constructed into Fv molecules and coupled to alternative ligands such as those illustrated in the cited article. Higgins J. Infect. Disease 166 (1992), 198-202, described a hetero-conjugate antibody composed of OKT3 cross-linked to an antibody directed to a specific sequence in the V3 region of GP120. Such hetero-conjugate antibodies can also be constructed using at least the variable regions contained in the antibody of the invention. Additional examples of specific antibodies include those described by Fanger, Cancer Treat. Res. 68 (1993), 181-194 and by Fanger, Crit. Rev. Immunol. 12 (1992), 101-124. Conjugates that are immunotoxins including conventional antibodies have been widely described in the art. The toxins may be coupled to the antibodies by conventional coupling techniques or immunotoxins containing protein toxin portions can be produced as fusion proteins. The antibodies of the present invention can be used in a corresponding way to obtain such immunotoxins. Illustrative of such immunotoxins are those described by Byers Seminars Cell. Biol. 2 (1991), 59-70 and by Fanger, Immunol. Today 12 (1991), 51-54.

The above described fusion protein may further comprise a cleavable linker or cleavage site for proteinases. These spacer moieties, in turn, can be either insoluble or soluble (Diener et al. Science 231 (1986), 148) and can be selected to enable drug release from the antigen at the target site. Examples of therapeutic agents which can be coupled to the antibodies and antigens of the present invention for immunotherapy are chemokines, homing molecules, drugs, radioisotopes, lectins, and toxins. The drugs which can be conjugated to the antibodies and antigens of the present invention depend on the disease context in which the conjugated molecules are intended to be used. For example, antibodies specific for targets useful in the treatment of tumor diseases can be conjugated to compounds which are classically referred to as anti-neoplastic drugs such as mitomycin C, daunorubicin, and vinblastine. In using radioisotopically conjugated antibodies or antigens of the invention for, e.g., tumor immunotherapy, certain isotopes may be more preferable than others depending on such factors as leukocyte distribution as well as stability and emission. Depending on the autoimmune response, some emitters may be preferable to others. In general, a and B particle emitting radioisotopes are preferred in immunotherapy. Preferred are short range, high energy a emitters such as ²¹²Bi. Examples of radioisotopes which can be bound to the antibodies or antigens of the invention for therapeutic purposes are ¹²⁵I, ¹³¹I, ⁹⁰Y, ⁶⁷Cu, ²¹²Bi, ²¹²At, ²¹¹Pb, ⁴⁷Sc, ¹⁰⁹Pd and ¹⁸⁸Re. Other therapeutic agents which can be coupled to the antibody or antigen of the invention, as well as ex vivo and in vivo therapeutic protocols, are known, or can be easily ascertained, by those of ordinary skill in the art. Non-limiting examples of suitable radionuclides for labeling are ¹⁹⁸Au, ²¹²Bi, ¹¹C, ¹⁴C, ⁵⁷Co, ⁶⁷Cu, ¹⁸F, ⁶⁷Ga, ⁶⁸Ga, ³H, ¹⁹⁷Hg, ¹⁶⁶Ho, ¹¹¹In, ^{113m}In, ¹²³I, ¹²⁵I, ¹²⁷I, ¹³¹I, ¹¹¹In, ¹⁷⁷Lu, ¹⁵O, ¹³N, ³²P, ³³P, ²⁰³Pb, ¹⁸⁶Re, ¹⁸⁸Re, ¹⁰⁵Rh, ⁹⁷Ru, ³⁵S, ¹⁵³Sm and ^{99m}Tc. Other molecules suitable for labeling are a fluorescent or luminescent dye, a magnetic particle, a metal, and a molecule which may be detected through a secondary enzymatic or binding step such as an enzyme or peptide tag. Commercial fluorescent probes suitable for use as labels in the present invention are listed in the Handbook of Fluorescent Probes and Research Products, 8th Edition, the disclosure contents of which are incorporated herein by reference. Magnetic particles suitable for use in magnetic particle-based assays (MPAs) may be selected from paramagnetic, diamagnetic, ferromagnetic and superpara-magnetic materials.

General methods in molecular and cellular biochemistry useful for diagnostic purposes can be found in such standard textbooks as Molecular Cloning: A Laboratory Manual, 3rd Ed. (Sambrook et al. Harbor Laboratory Press 2001); Short Protocols in Molecular Biology, 4th Ed. (Ausubel et al. eds., John Wiley & Sons 1999); Protein Methods (Bollag et al. John Wiley & Sons 1996). Reagents, detection means and kits for diagnostic purposes are available from commercial vendors such as Pharmacia Diagnostics, Amersham, BioRad, Stratagene, Invitrogen, and Sigma-Aldrich as well as from the sources given in any one of the references cited herein, in particular patent literature.

### Treatment and Drugs:

As used herein, the terms “treat” or “treatment” refer to both therapeutic treatment and prophylactic or preventative measures, wherein the object is to prevent or slow down (lessen) an undesired physiological change or disorder, such as the development of an autoimmune and/or autoinflammatory disease. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the manifestation of the condition or disorder is to be prevented.

If not stated otherwise the term “drug,” “medicine,” or “medicament” are used interchangeably herein and shall include but are not limited to all (A) articles, medicines and preparations for internal or external use, and any substance or mixture of substances intended to be used for diagnosis, cure, mitigation, treatment, or prevention of disease of either man or other animals; and (B) articles, medicines and preparations (other than food) intended to affect the structure or any function of the body of man or other animals; and (C) articles intended for use as a component of any article specified in clause (A) and (B). The term “drug,” “medicine,” or “medicament” shall include the complete formula of the preparation intended for use in either man or other animals containing one or more “agents,” “compounds”, “substances” or “(chemical) compositions” as and in some other context also other pharmaceutically inactive excipients as fillers, disintegrants, lubricants, glidants, binders or ensuring easy transport, disintegration, disaggregation, dissolution and biological availability of the “drug,” “medicine,” or “medicament” at an intended target location within the body of man or other animals, e.g., at the skin, in the stomach or the intestine. The terms “agent,” “compound” or “substance” are used interchangeably herein and shall include, in a more particular context, but are not limited to all pharmacologically active agents, i.e. agents that induce a desired biological or pharmacological effect or are investigated or tested for the capability of inducing such a possible pharmacological effect by the methods of the present invention.

Examples of “anti-rheumatic drugs” and immunosuppressive drugs include chloroquine, hydroxycloroquine, myocrisin, auranofin, sulfasalazine, methotrexate, leflunomide, etanercept, infliximab (plus oral and subcutaneous methotrexate), adalimumab etc., azathioprine, D-penicilamine, gold salts (oral), gold salts (intramuscular), minocycline, cyclosporine including cyclosporine A and topical cyclosporine, tacrolimus, mycophenolate mofetil, cyclophosphamide, staphylococcal protein A (Goodyear and Silverman J. Exp. Med., 197 (2003), 125-39), including salts and derivatives thereof, etc.

Examples of “non-steroidal anti-inflammatory drugs” or “NSAIDs” include aspirin, acetylsalicylic acid, ibuprofen and ibuprofen retard, fenoprofen, piroxicam, flurbiprofen, naproxen, ketoprofen, naproxen, tenoxicam, benorylate, diclofenac, naproxen, nabumetone, indomethacin, ketoprofen, mefenamic acid, diclofenac, fenbufen, azapropazone, acemetacin, tiaprofenic acid, indomethacin, sulindac, tolmetin, phenylbutazone, diclofenac and diclofenac retard, cyclooxygenase (COX)-2 inhibitors such as GR 253035, MK966, celecoxib (CELEBREX®; 4-(5-(4-methylphenyl)-3-(trifluoromethyl)-1H-pyrazol-1-yl), benzenesulfon-amide and valdecoxib (BEXTRA®), and meloxicam (MOBIC®), including salts and derivatives thereof, etc. Preferably, they are aspirin, naproxen, ibuprofen, indomethacin, or tolmetin. Such NSAIDs are optionally used with an analgesic such as codenine, tramadol, and/or dihydrocodinine or narcotic such as morphine.

By “subject” or “individual” or “animal” or “patient” or “mammal,” is meant any subject, particularly a mammalian subject, e.g., a human patient, for whom diagnosis, prognosis, prevention, or therapy is desired.

### Pharmaceutical Carriers:

Pharmaceutically acceptable carriers and administration routes can be taken from corresponding literature known to the person skilled in the art. The pharmaceutical compositions of the present invention can be formulated according to methods well known in the art; see for example Remington: The Science and Practice of Pharmacy (2000) by the University of Sciences in Philadelphia, ISBN 0-683-306472, Vaccine Protocols. 2nd Edition by Robinson et al., Humana Press, Totowa, N.J., USA, 2003; Banga Therapeutic Peptides and Proteins: Formulation, Processing, and Delivery Systems. 2nd Edition by Taylor and Francis. (2006), ISBN: 0-8493-1630-8. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well-known conventional methods. These pharmaceutical compositions can be administered to the subject at a suitable dose. Administration of the suitable compositions may be effected by different ways. Examples include administering a composition containing a pharmaceutically acceptable carrier via oral, intranasal, rectal, topical, intraperitoneal, intravenous, intramuscular, subcutaneous, subdermal, transdermal, intrathecal, and intracranial methods. Aerosol formulations such as nasal spray formulations include purified aqueous or other solutions of the active agent with preservative agents and isotonic agents. Such formulations are preferably adjusted to a pH and isotonic state compatible with the nasal mucous membranes. Pharmaceutical compositions for oral administration, such as single domain antibody molecules (e.g., “Nanobodies™”) etc are also envisaged in the present invention. Such oral formulations may be in tablet, capsule, powder, liquid or semi-solid form. A tablet may comprise a solid carrier, such as gelatin or an adjuvant. Formulations for rectal or vaginal administration may be presented as a suppository with a suitable carrier; see also O'Hagan et al. Nature Reviews, Drug Discovery 2 (2003), 727-735. Further guidance regarding formulations that are suitable for various types of administration can be found in Remington's Pharmaceutical Sciences, Mace Publishing Company, Philadelphia, Pa., 17th ed. (1985) and corresponding updates. For a brief review of methods for drug delivery see Langer Science 249 (1990), 1527-1533.

### Dosage Regimen:

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as anti-tumor agents and cytotoxic drugs, depending on the intended use of the pharmaceutical composition.

In addition, co-administration or sequential administration of other agents may be desirable. A therapeutically effective dose or amount refers to that amount of the active ingredient sufficient to ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Preferably, the therapeutic agent in the composition is present in an amount sufficient for preventing inflammation or suppression of the immune response.

These and other embodiments are disclosed and encompassed by the description and examples of the present invention. Further literature concerning any one of the materials, methods, uses and compounds to be employed in accordance with the present invention may be retrieved from public libraries and databases, using for example electronic devices. For example the public database “PubMed” may be utilized, which is hosted by the National Center for Biotechnology Information and/or the National Library of Medicine at the National Institutes of Health. Further databases and web addresses, such as those of the European Bioinformatics Institute (EBI), which is part of the European Molecular Biology Laboratory (EMBL) are known to the person skilled in the art and can also be obtained using internet search engines. An overview of patent information in biotechnology and a survey of relevant sources of patent information useful for retrospective searching and for current awareness is given in Berks TIBTECH 12 (1994), 352-364.

The above disclosure generally describes the present invention. Several documents are cited throughout the text of this specification. Full bibliographic citations may be found at the end of the specification immediately preceding the claims. The contents of all cited references (including literature references, issued patents, published patent applications as cited throughout this application, including the disclosure in the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only.

### Examples

The Examples 1 to 10 which follow and corresponding FIGS. 1 to 18 further illustrate the invention, but should not be construed to limit the scope of the invention in any way. Detailed descriptions of conventional methods, such as those employed herein can be found in the cited literature; see also “The Merck Manual of Diagnosis and Therapy” Seventeenth Ed. ed. by Beers and Berkow (Merck & Co., Inc., 2003). The practice of the present invention will employ, unless otherwise indicated, conventional techniques of cell biology, cell culture, molecular biology, transgenic biology, microbiology, recombinant DNA, and immunology, which are within the skill of the art.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds.); Immobilized Cells And Enzymes (IRL Press, 1986); Perbal, A Practical Guide To Molecular Cloning (1984); the treatise, Methods In Enzymology (Academic Press, Inc., N.Y.); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, Promega and Clontech. General techniques in cell culture and media collection are outlined in Large Scale Mammalian Cell Culture (Hu et al. Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al. Bioprocess Technol. 19 (1990), 251.

### Example 1: Detection of Human Cytokine Specific Antibodies in the Serum of Patients by LIPS Assay, Cloning and Recombinant Expression of Candidate Anti-IL-20 Antibodies

LIPS assay was used for differential analysis of IL-20 antibodies in the sera of the patients suffering from the genetic condition APECED (Autoimmune polyendocrinopathy candidiasis epidermal dysplasia, also called Autoimmune polyendocrinopathy type 1 (APS1). IL-20 was fused with Gaussia luciferase at the N-terminus and expressed by transient transfection of HEK293 cells as described in Example 10 on page 158 and Example 15 on pages 165-167 in applicant's international application WO2013/098419, the disclosure content of which is incorporated herein by reference, using primers indicated in Table 3, infra.

This protocol describes the experimental procedures for the detection of antibodies by Luciferase Immunoprecipitation System (LIPS) technique.

### Materials:

MultiScreen HTS Filter Plates, Millipore #MSBVN1B50
Recombinant Protein G Agarose Beads, Exalpha #X1197
Buffer A (50 mM Tris, pH 7.5, 100 mM NaCl, 5 mM MgCl₂, 1% Triton X-100)
Luciferase Assay System, Promega #E1501
Gaussia Luciferase Assay Reagent, Targeting Systems GAR-2B
Luc-antigen (store at −80° C., concentration (LU/ml))
PBS

### Hardware:

Rotary shaker
MultiScreenHTS Vacuum Manifold, Millipore #MSVMHTS00
1450 MicroBeta TriLux Liquid Scintillation Counter & Luminometer, Perkin Elmer
Victor X5 Multilabel Plate Reader, Perkin Elmer
Multichannel pipette: Biohit 30-300 μl
Eppendorf Centrifuge 5415D

### Methods:

Resuspend Protein G Agarose carefully, take the necessary amount (per one plate approximately 200 μl of suspension that makes 100 μl drained medium). Centrifuge 300 g 5 min, check the drained volume and wash once with buffer A (3× volume), 300 g 5 min. Resuspend in Buffer A regarding that 25 μl of suspension would contain 1 μl of drained Protein G Agarose. (It would be useful to aliquot Protein G Agarose to tubes in amounts that are usually needed.)
Add 25 μl/well 1:100 dilution of patient sera (diluted in Buffer A).
Add 25 μl/well Protein G Agarose suspension.
Incubate the plate on a rotary shaker for 1 hour at room temperature.
Dilute the Luc-antigen so that 10⁶ (5*10⁵) light units (LU) would be added in 50 μl of Buffer A. Add 50 μl/well Luc-antigen dilution.
Incubate the plate on a rotary shaker for 1 hour at room temperature.
Wash the plate on the vacuum manifold. Each well is washed 5 times with 200 μl of Buffer A, followed by two times with 200 μl of PBS. Blot the plate dry using a stack of paper towels or filter paper making sure to remove moisture on the top and bottom of the plate.
Using Gaussia-constructs, add Gaussia substrate 10 μl/well and shake for 2 sec. Read the luminescence immediately during 5 sec according to instructions in the instrument manual. Using Firefly-constructs, add Luciferase substrate 20 μl/well and shake for 2 sec. Read the luminescence immediately during 5 sec according to instructions in the instrument manual.

TABLE 3
IL-20 with Gaussia luciferase at its N-terminus. Full-length IL-20 without signal
sequence (25 -176 aa) was cloned into fusion with Gaussia luciferase gene at the IL-20 N-
terminus using pGaussia1 (Molecular Pathology, University of Tartu, Estonia) mammalian
expression vector (into EcoRI and NotI sites). After the transfection into HEK 293 cells, cell
culture supernatant was used as antigen in luciferase-based immunoprecipitation assay.

| | | Primer | |
|---|---|---|---|
| Gene | AA | name | Sequence and SEQ ID NO: |
| IL-20 | 25-176 | IL20-F | TTTGGATCCTA*CTGAAGACACTCAATTTGGGAAGCTG |
| | | | (IL20 specific 26 bp, flanked with EcoRI site), SEQ ID NO: 33 |
| | | IL20-R | TTTGCGGCCGC*CTATTCTGTCTCCTCCATCCATTGCA |
| | | | (IL20 specific 26 bp, flanked with NotI site), SEQ ID NO: 34 |

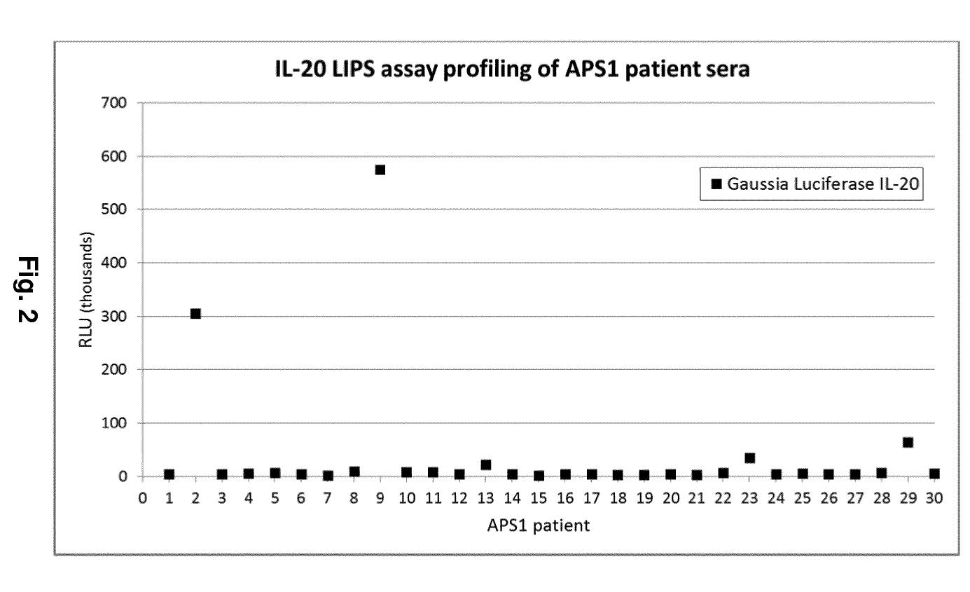

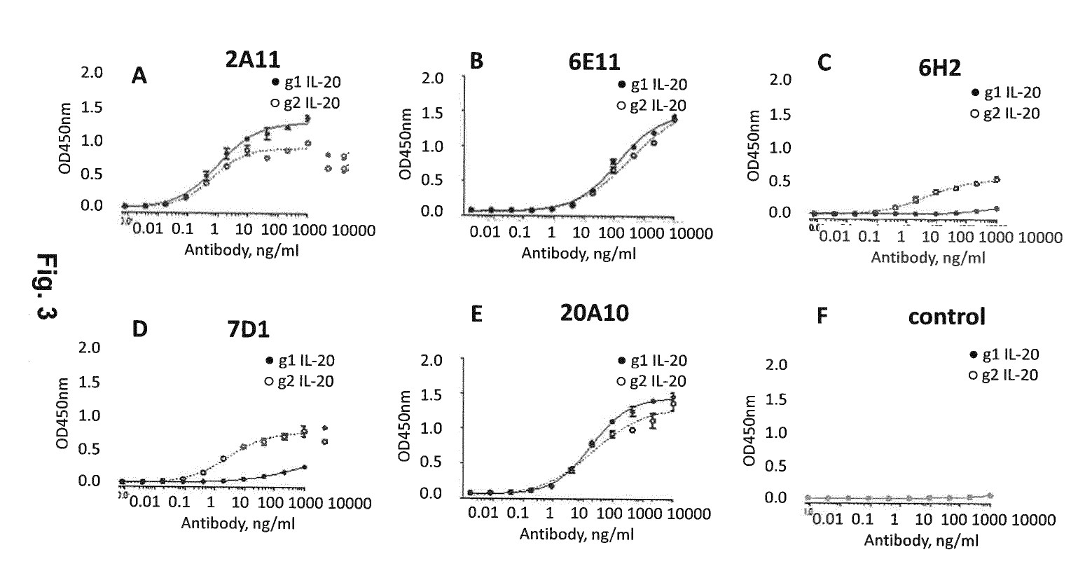

Altogether sera from 30 patients, presented by codes from APS1-1 to APS1-30 were used in the assays. As shown in FIG. 2 sera from patient APS1-9 revealed the highest titers of anti-IL-20 antibodies. Accordingly, patient APS1-9 was used as the source of memory B cells for cloning of candidate anti-IL-20 antibodies. Memory B cell isolation, culture and antibody isolation were carried out as described in the international applications WO 2013/098419 and WO 2013/098420 with the difference that specificity of the antibodies isolated and analyzed was directed towards IL-20 as defined hereinabove and below instead of IL-17 and IL-22, which were specifically used in the mentioned international applications; see Examples sections therein, in particular Examples 1 and 2 on pages 117 to 120 and Example 17 on pages 168-171 of WO 2013/098419 and Examples 1 to 4 on pages 27 to 31 of WO 2013/098420, the disclosure content of which is incorporated herein by reference.

### Example 2: EC 50 ELISA Determination of Candidate Anti-IL-20 Antibodies

The molecular cloning of human antibodies of the present invention and subsequent antibody production and purification were performed as described in the international application WO 2013/098419, see the Examples section of the application and in particular Examples 1 to 3 on pages 117-120 therein, the disclosure content of which is incorporated herein by reference. EC 50 binding of exemplary anti-IL-20 antibodies of the present invention to fusion protein of human IL-20 with Gaussia luciferase (g1 IL-20 and g2 IL-20), were determined by GLuc-sandwich ELISA. Plates were coated with anti-Gluc Ab (1 μg/ml), Gluc antigens were bound and serial dilutions of MABs were bound for 2 h. The plates were subsequently washed and binding of MABs was detected with anti-human HRP-conjugated secondary antibody (Jackson ImmunoResearch, Europe Ltd., Cambridgeshire, UK). Concentrations of MAB resulting in half of maximal binding to respective antigens (EC 50, ng/ml) were calculated using Prism 4 GraphPad software on sigmoidal dose-response curves (variable slope, 4 parameters) obtained by plotting the log of the concentration versus OD 450 nm measurements for the results see FIG. 3 and Table 4 below.

TABLE 4
Summary of EC 50 values of binding of MABs to g1 IL-20 and g2
IL-20. MABs 2A11, 6E11, 6H2, 7D1 and 20A10 did bind to g1 IL-20
and g2 IL-20. MAB 7D1 bound to g1 IL-20 only at very high
concentrations indicating that EC 50 binding is higher than 5 μg/ml.

| | EC₅₀ (ng/ml) | | | | |
|---|---|---|---|---|---|
| | 2A11 | 6E11 | 6H2 | 7D1 | 20A10 |
| g1 IL-20 | 9.8 | 128 | 8548 | 5868 | 18.6 |
| g2 IL-20 | 6.5 | 347 | 42.2 | 27.1 | 21.0 |

### Example 3: Human-Derived Anti-IL-20 Monoclonal Antibodies Neutralize Recombinant IL-20 and IL-20 Receptor Mediated STAT3 Activation

The neutralizing assays are carried out with cell lines that respond to the studied cytokine. The ligand binding to receptor in general activates a corresponding signaling pathway, translocation of transcription factors to the nucleus and upregulates responder gene transcription, translation and if applicable product secretion. The cytokine concentration used is selected from the beginning of the linear part of the dose-response curve to maximize the sensitivity of the assay. To test the neutralizing capacity of antibodies the optimal concentration of the target cytokine is preincubated with serial dilutions of serum, supernatant or purified antibody samples. The results are expressed as titer or concentration of antibody that show the value half-way between the positive and negative controls.

In the first set of experiments human-derived anti-IL-20 monoclonal antibodies were subjected to a phospho-STAT3 assay for assessing their neutralizing activity towards recombinant IL-20-mediated STAT3 activation in HEK 293T MSR cells transiently expressing Type I and Type II IL-20 receptors (FIG. 4).

### Phospho-STAT3 Assay

20,000 HEK 293T MSR cells (Cat. No. R79507, Invitrogen, Carlsbad, Calif., USA) were seeded into 96-well tissue culture plates (Corning Inc., Corning, N.Y., USA). The following day, cells were co-transfected with either IL20RA-Myc-DDK and IL20RB-Myc-DDK (Type I IL-20 receptor; Cat. No. RC212546 and RC213197, OriGene, Rockville, Md., USA) or IL22RA (Cat. No. SC322566, OriGene) and IL20RB-Myc-DDK expression plasmids (Type II IL-20 receptor). The day following transfection, recombinant IL-20 was mixed with anti-IL-20 mAbs or control IgG and pre-incubated for one hour at 37° C. After pre-incubation, the mixtures were used to stimulate HEK 293T MSR cells transiently expressing IL-20 receptors for 40 minutes at 37° C. Following stimulation, cells were lysed with CelLytic™ M lysis buffer supplemented with protease and phosphatase inhibitors (Cat. No. C2978, P5726, P0044, P8340, SIGMA-ALDRICH, St. Louis, Mo., USA) and the collected lysates were cleared at 13,000 RPM, 4° C. in a tabletop centrifuge. Lysates were subjected to reducing SDS-PAGE and blotted onto nitrocellulose membranes. Membranes were blocked with a buffer containing 0.25% bovine gelatin, 150 mM NaCl, 5 mM EDTA, 50 mM Tris/HCl pH 7.5, 0.05% Triton X-100 for one hour at room temperature, followed by incubation with rabbit monoclonal antibodies against phosphorylated STAT3 (Tyr705, diluted 1:2000 in blocking buffer, Cat. No. 9145, Cell Signaling Technology, Danvers, Mass., USA) or alpha-tubulin (diluted 1:2500, Cat. No. 2125, Cell Signaling Technology) at 4° C. over night. On the next day, blots were washed three times with blocking buffer followed by incubation with horseradish peroxidase-linked secondary antibodies against rabbit IgG (diluted 1:20,000 in blocking buffer, Cat. No. 111-035-144, Jackson ImmunoResearch, West Grove, Pa., USA). After three additional washing steps, an ECL substrate was added (Cat. No. 34087, Thermo Fisher Scientific, Rockford, Ill., USA) and reactive bands were visualized via autoradiography. Bound antibodies were removed by incubation in Restore Western Blot Stripping Buffer (Cat. No. 21059, Thermo Fisher Scientific) and a rabbit monoclonal anti-STAT3 serum was used to visualize total STAT3 levels (diluted 1:1000, Cat. No. 12640, Cell Signaling Technology). Expression of IL-20 receptor subunits was visualized with a mouse monoclonal anti-DDK serum (diluted 1:1000, Cat. No. TA50011, OriGene) and with rabbit polyclonal sera against IL-20RA and IL-22RA (diluted 1:500 and 1:1000, respectively, Cat. No. 06-1073 and 06-1077, Millipore, Billerica, Mass., USA).

TABLE 5
List of recombinant proteins used in the neutralization
assays (Interleukins).

| Protein | Provider | Cat. No. |
|---|---|---|
| rhIL-20 | R&D Systems | 1102-IL/CF |
| rmIL-20 | R&D Systems | 1204-ML/CF |
| g2 IL-22 | fusion protein of human IL-22 with Gaussia | |
| | luciferase | |
| g1 IL-20 | fusion protein of human IL-20 with Gaussia | |
| | luciferase | |

As a control, rhIL-20 and rmIL-20 induce the dose-dependent phosphorylation of STAT3 in HEK 293T MSR cells transiently expressing Type I or Type II IL-20 receptors. Detection of total and phosphorylated STAT3 and IL-20RB-Myc-DDK levels (FIG. 4A).

As shown in FIG. 4B antibody 20A10 and 7D1 as well as 2A11 are the most potent neutralizing antibodies. However, this is partly in contrast what might have been expected from the results obtained in Example 2 and shown in FIG. 3 where for example antibody 6E11 displayed a higher binding affinity than antibody 7D1 and antibody 2A11 performed substantially better than antibody 7D1.

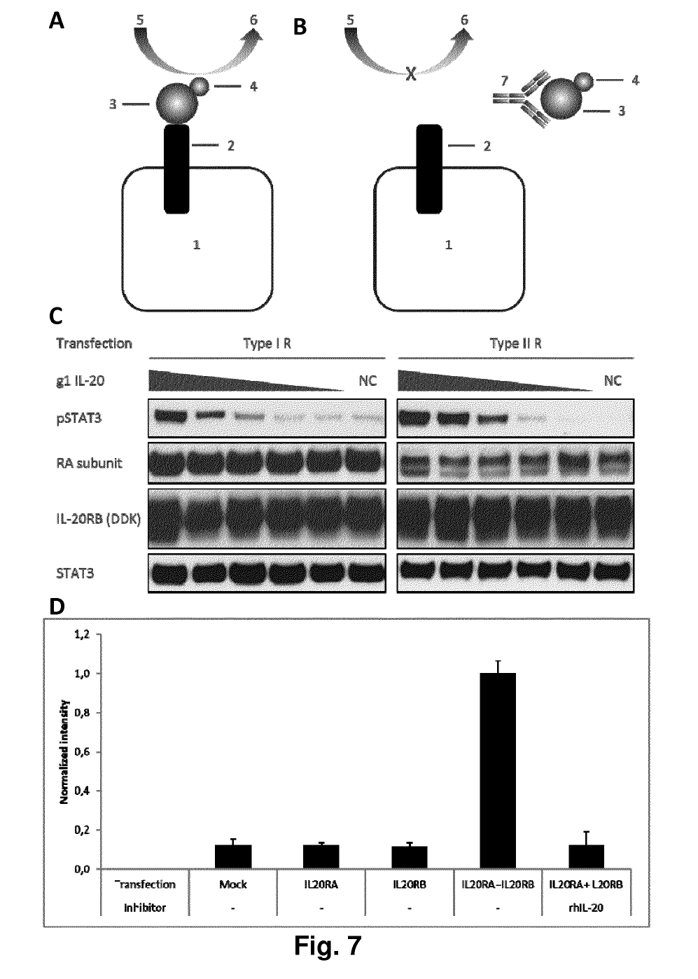

Next, a titration of 20A10 against rmIL-20 in the pSTAT3 Western blot assay was performed. HEK 293T MSR cells transiently expressing Type I IL-20 receptor were either left untreated or stimulated with 25 ng/ml rmIL-20 in the absence of antibodies (−) or in the presence (+) of exemplary antibody 20A10 as indicated (FIG. 6A). Cell lysates were subjected to SDS-PAGE and pSTAT3 levels were visualized in Western blots. Total STAT3 levels serve as loading control. By plotting a dose-response curve and examining the titration of IL-20 antibody 20A10 on reversing IL-20 activity an IC 50 of 1,867 ng/ml was calculated which supports the high neutralization activity of MAb 20A10 against rmIL-20 binding to Type I IL-20 receptors (FIG. 6B).

### Example 4: Human-Derived Anti-IL-20 Monoclonal Antibodies Neutralize rhIL-20 and IL-20 Receptor Mediated Induction of KZ136-NLuc Reporter Gene

In view of the inconsistency of the results of the EC 50 ELISA determination of candidate anti-IL-20 antibodies obtained in Example 2 and shown in FIG. 3 and the results of the phospho-STAT3 assay obtained in Example 3 and shown in FIG. 4B, supra, and in order to validate the efficacy of candidate anti-IL-20 antibodies 7D1, 20A10 and 2A11 in neutralizing IL-20-mediated induction of IL-20 receptors a KZ136-NLuc reporter neutralization assay was established.

### KZ136-NLuc Reporter Assay

### Generation of the KZ136-NLuc Reporter Construct

The KZ136 reporter construct as initially described by Poulsen and colleagues (Poulsen et al., Signal Transduction via the Mitogen-activated Protein Kinase Pathway Induced by Binding of Coagulation Factor VIIa to Tissue Factor, J B C, 1998) encodes an inducible firefly luciferase, preceded by a fragment of the c-fos promoter (nucleotides 649-747, GenBank™ accession number K00650). Immediately upstream, the construct features four STAT binding elements of the c-fos, p21WAF1,β-casein, and the Fcg RI genes, together with a serum response element. The original KZ136 construct was primarily used for the generation of stable cell lines. The KZ136-NLuc reporter described herein encodes the considerably brighter Nano luciferase and is suited for transient co-transfection experiments. A construct containing the KZ136 promoter sequence flanked by NheI and XhoI restriction sites was generated via gene synthesis. The KZ136 promoter sequence was excised using NheI and XhoI double restriction, followed by ligation into the Nano luciferase-encoding pNL2.1 target vector (Cat. No. N1061, Promega) which was digested with the same restriction enzymes (see scheme of the construct in FIG. 5A).

### KZ136-NLuc Reporter Neutralization Assay

10,000 HEK 293T MSR cells were seeded in white half area 96-well tissue culture plates (Cat. No. 3688, Corning Inc.). The following day, cells were co-transfected with 20 ng of KZ136-Nano Luciferase reporter and 80 ng IL-20 receptor constructs using Fugene HD (Cat. No. E2311, Promega, Madison, Wis., USA). The day following transfection, cells were stimulated for 24 hours with medium containing mixtures of 120 ng/ml recombinant human (rh) IL-20 (FIG. 5C, D) or 200 ng/ml recombinant mouse (rm) IL-20 (FIG. 5E) with or without anti-IL-20 mAbs or control human IgG (huIgG) that had been pre-incubated for one hour at 37° C. After 24 hours of stimulation, the luciferase assay was developed according to the manufacturer's instructions (Cat. No. N1130, Promega).

As a control rhIL-20 and rmIL-20 induce KZ136-NLuc reporter constructs in HEK 293T MSR cells transiently transfected with Type I IL-20 receptors and KZ136-NLuc (FIG. 5B). IC 50 analysis of exemplary human-derived IL-20 mAbs 20A10, 7D1 and 2A11 by KZ136-NLuc reporter assay using rhIL-20 (FIG. 5C, D) or rmIL-20 (FIG. 5E). The results are summarized in FIG. 5F, demonstrating the IL-20 neutralizing capacity of 20A10. As already seen in the phospho-STAT3-assay (FIG. 4B), antibody 20A10 showed the highest neutralization capacity compared to antibodies 7D1 and 2A11 with a higher efficacy towards rhIL-20 binding to Type I IL-20 receptors (FIG. 5F, left column).

### Example 5: Human-Derived Anti-IL-20 Monoclonal Antibodies Neutralize Binding of IL-20 to IL-20 Receptors in a Chemiluminescent Cellular Binding Assay

Since the LIPS assay has not been predictive for the antagonizing, i.e. neutralizing activity of the candidate antibodies, a new assay for the assessment of the binding of a ligand of interest to cells expressing the relevant receptor(s) has been developed in accordance with the present invention for use in determining whether candidate antibodies directed against the ligand prevent its ability to bind to the respective receptor(s) on the cells.

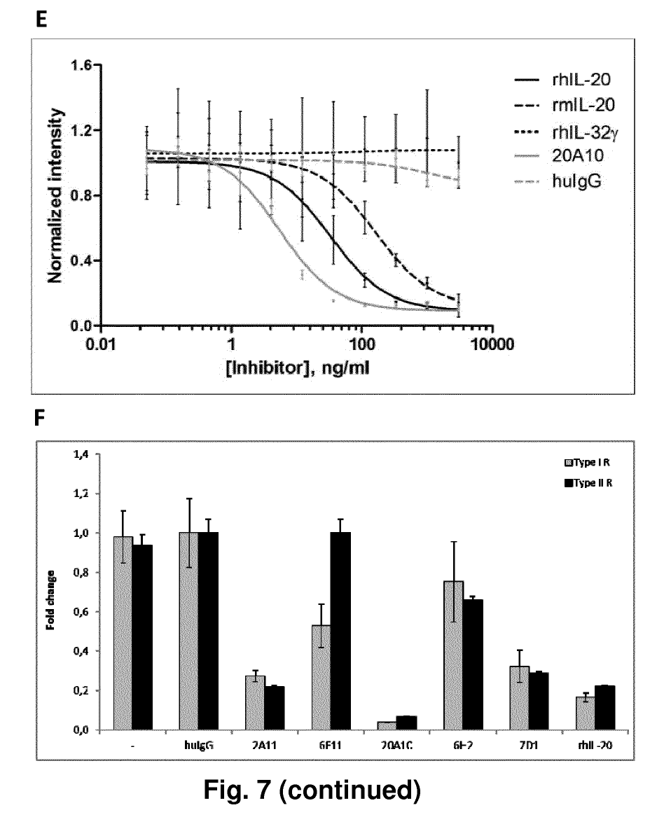

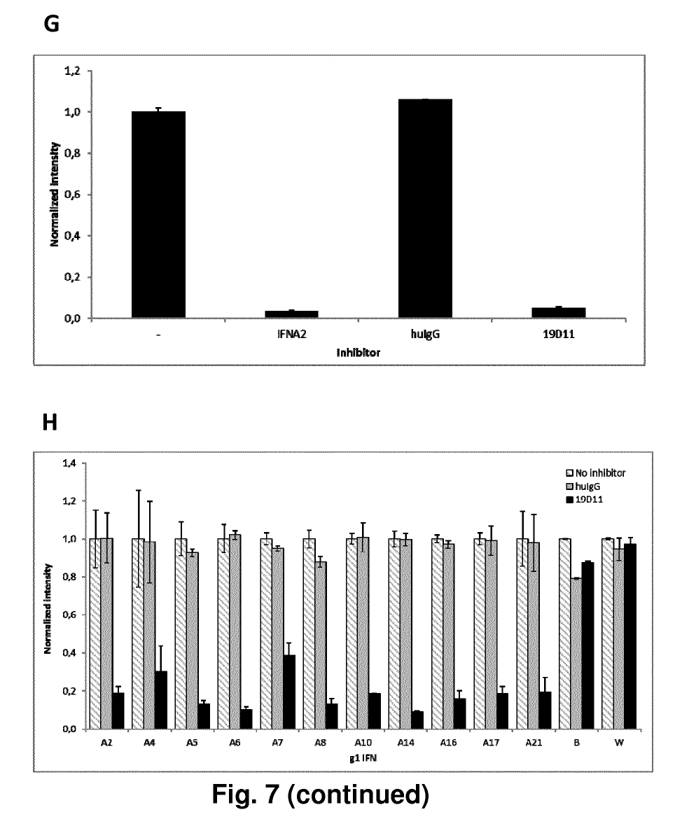

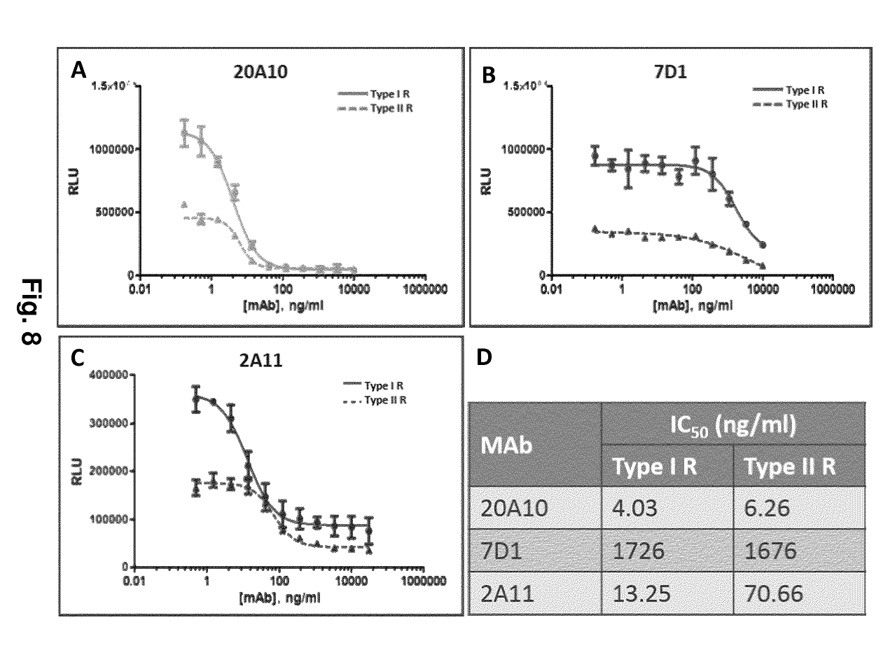

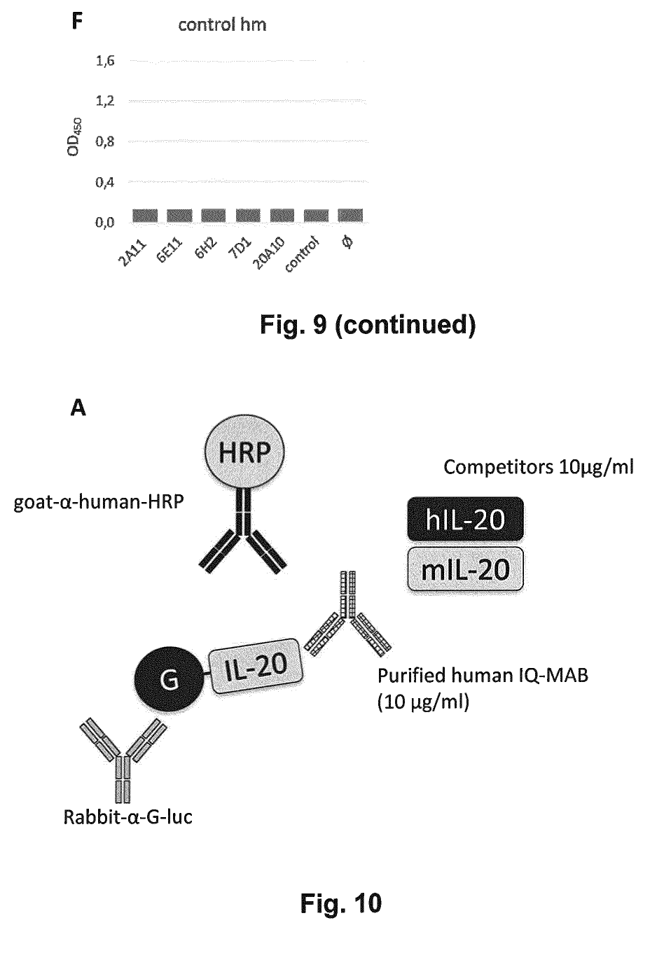

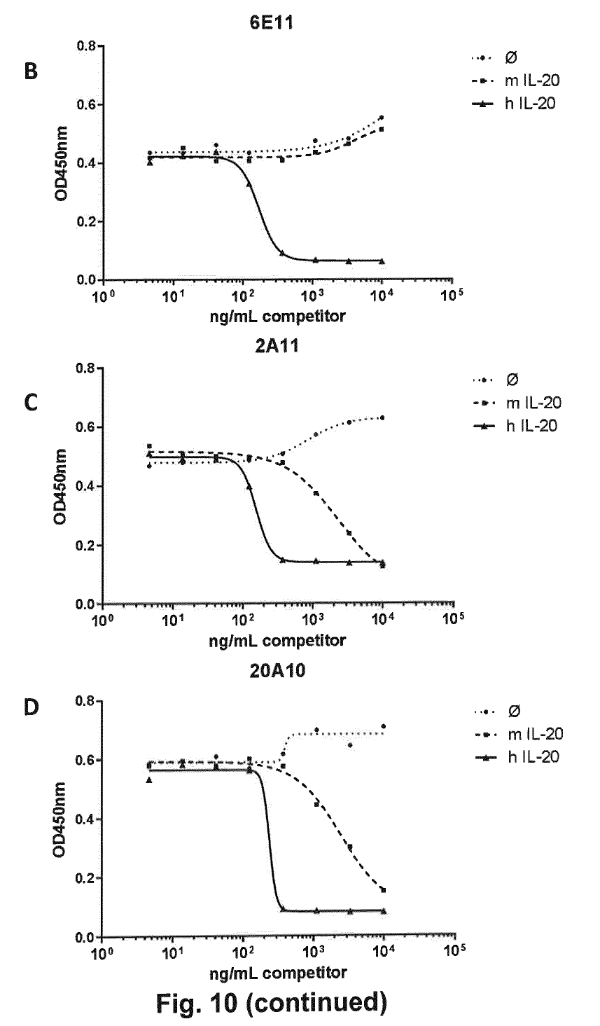

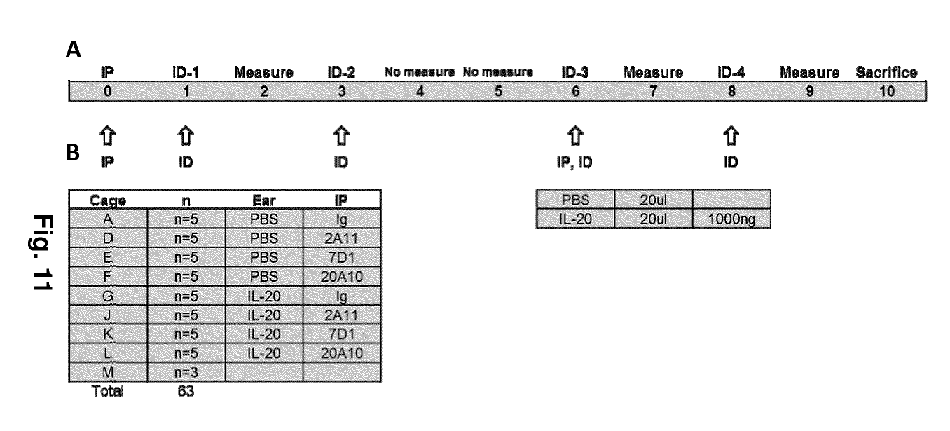

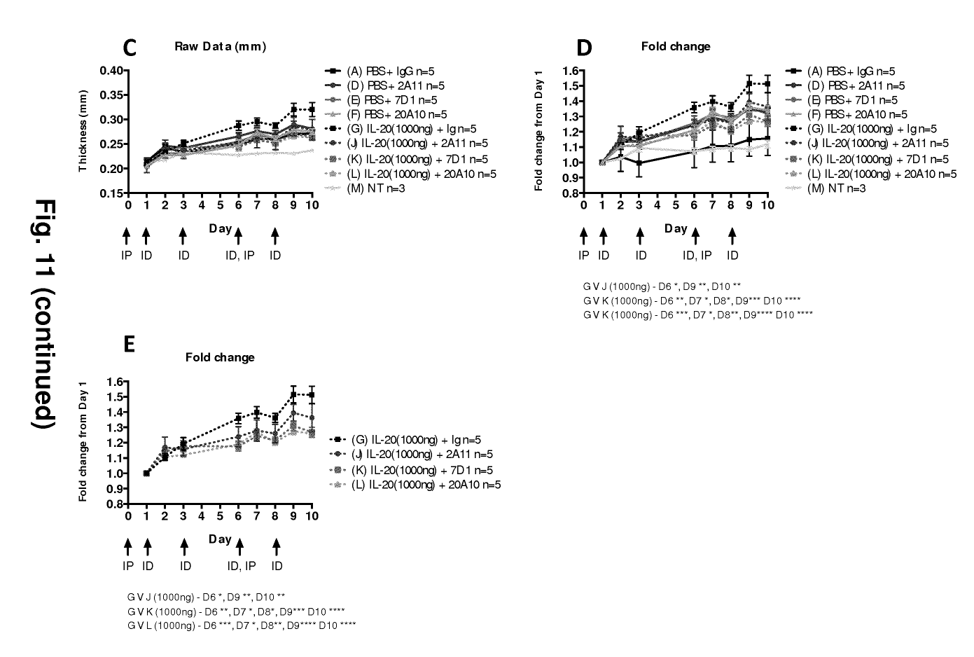

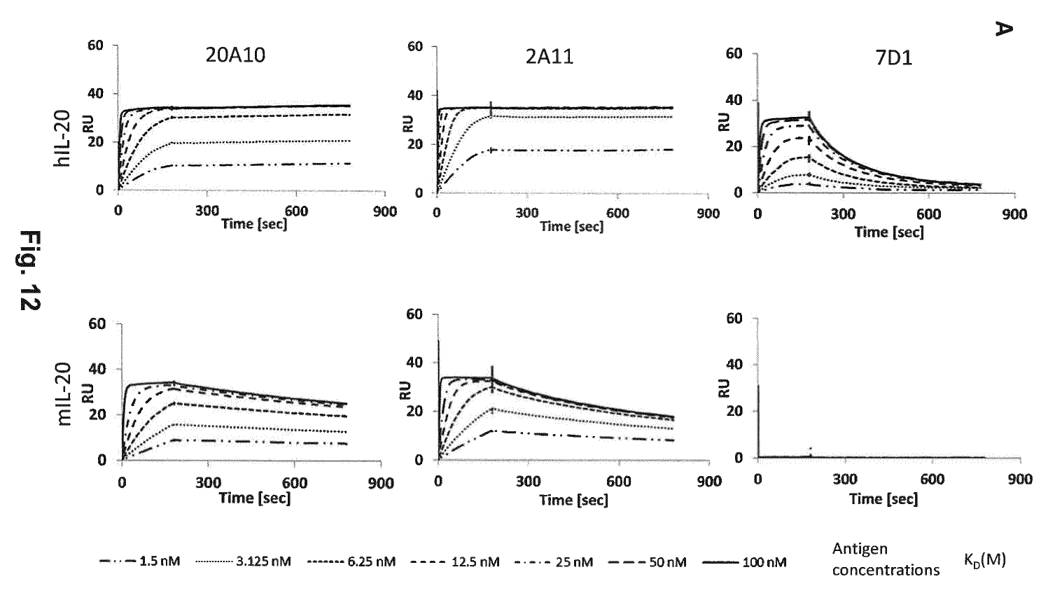

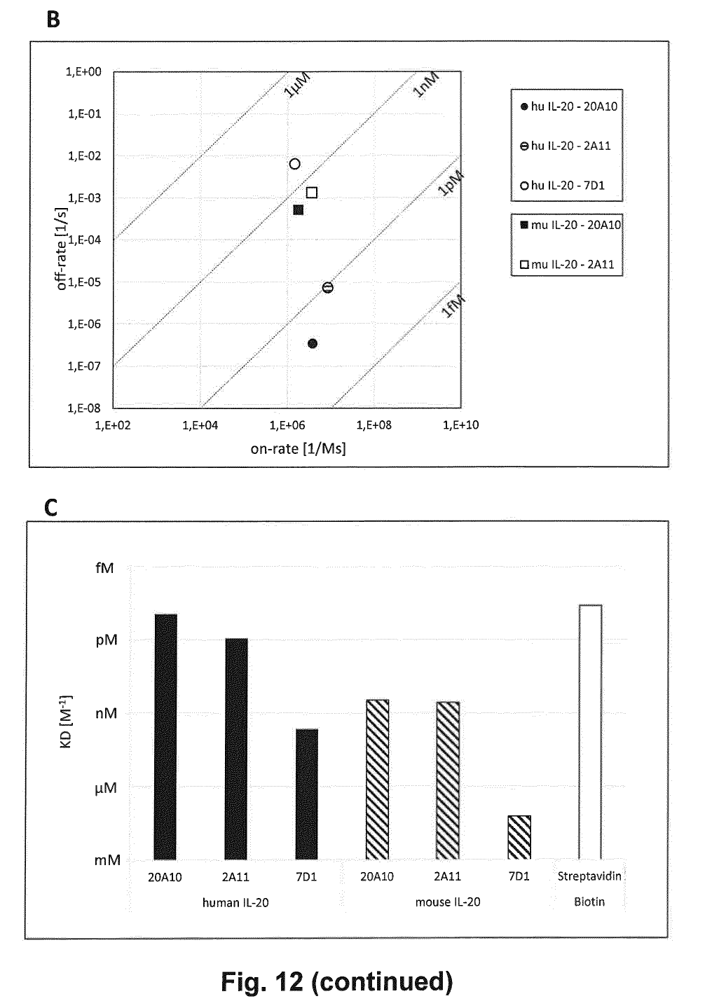

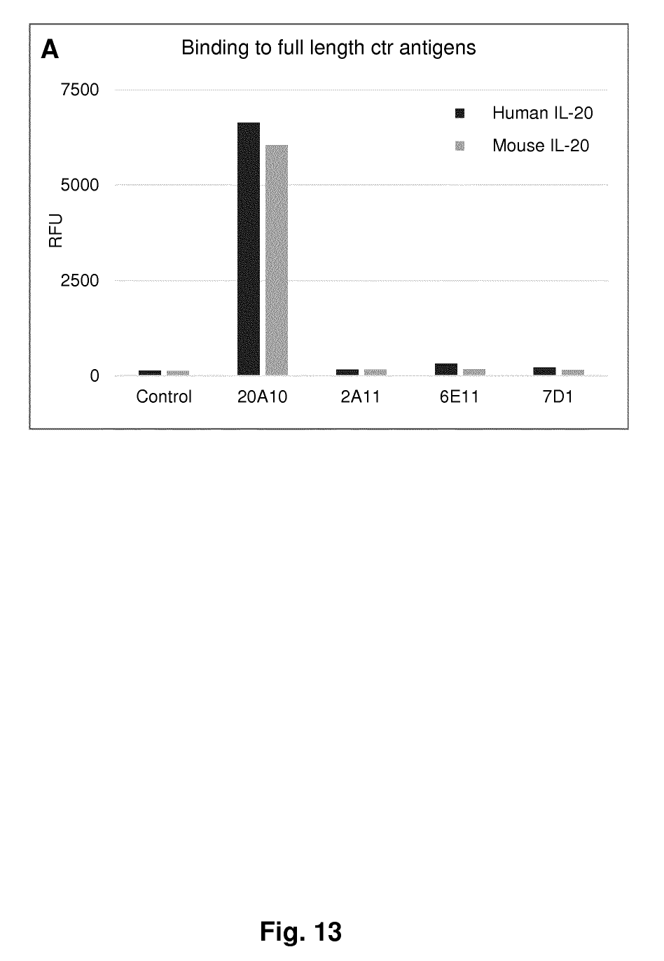

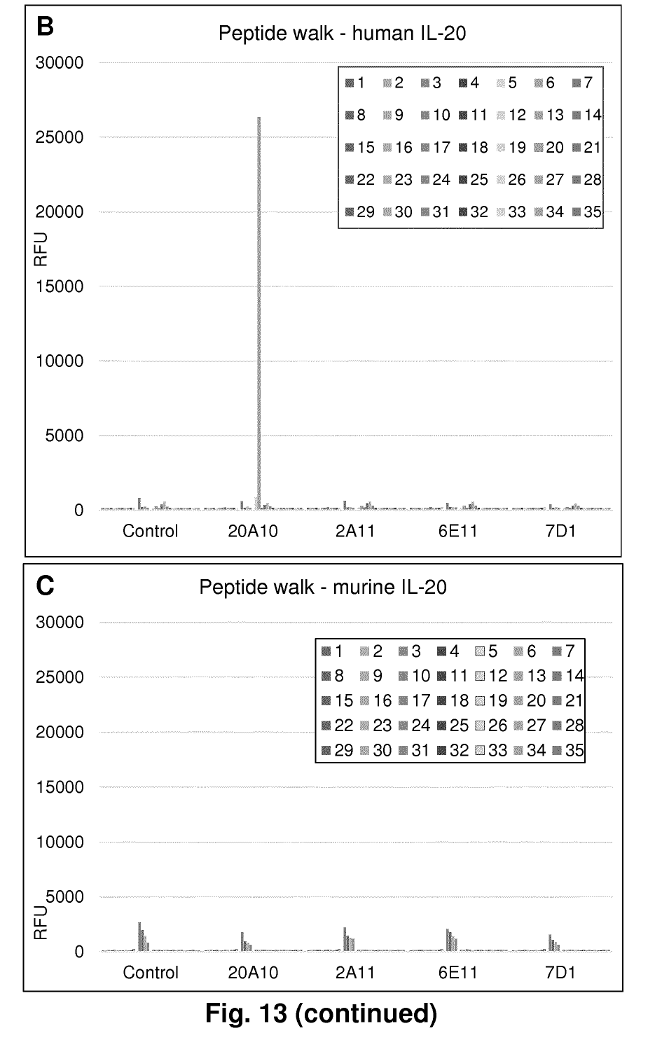

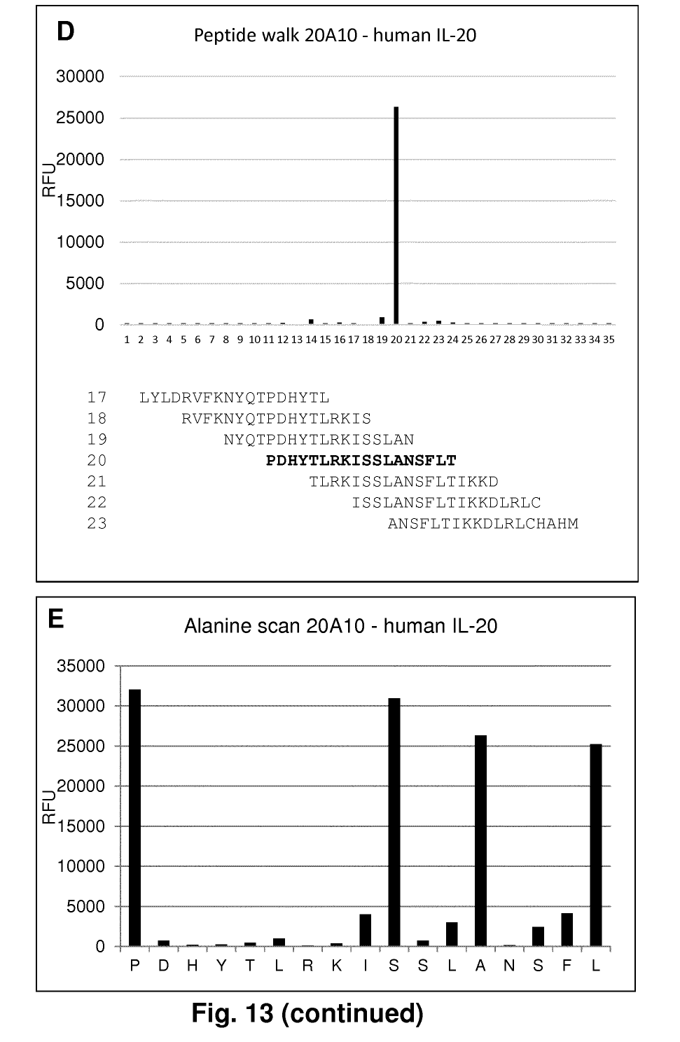

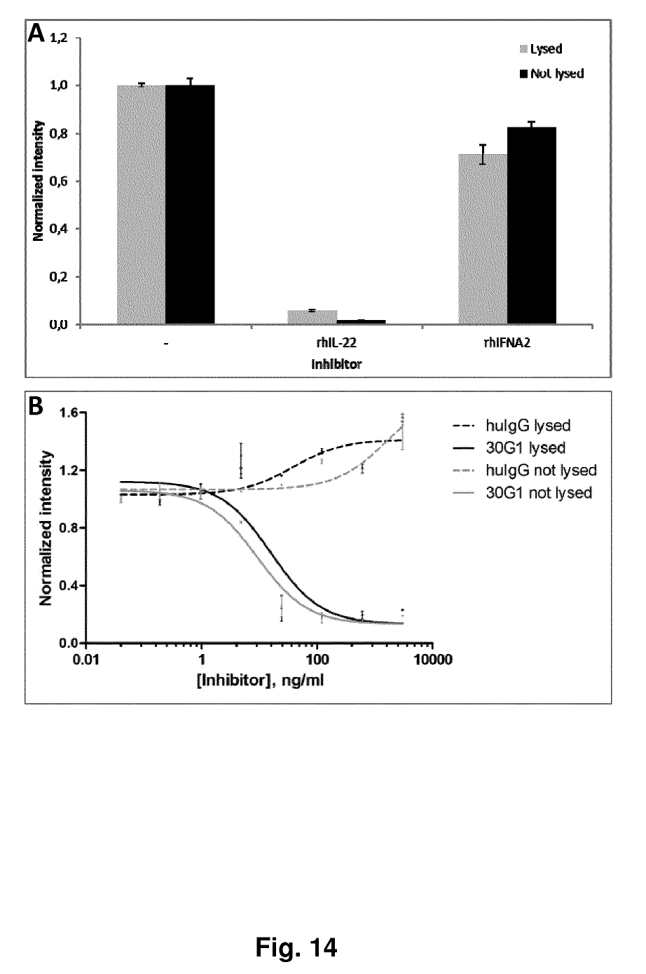

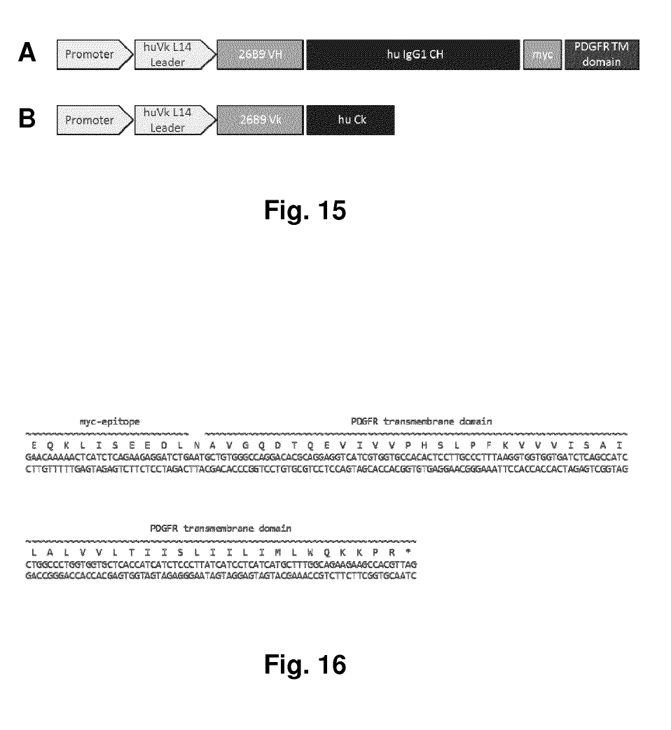

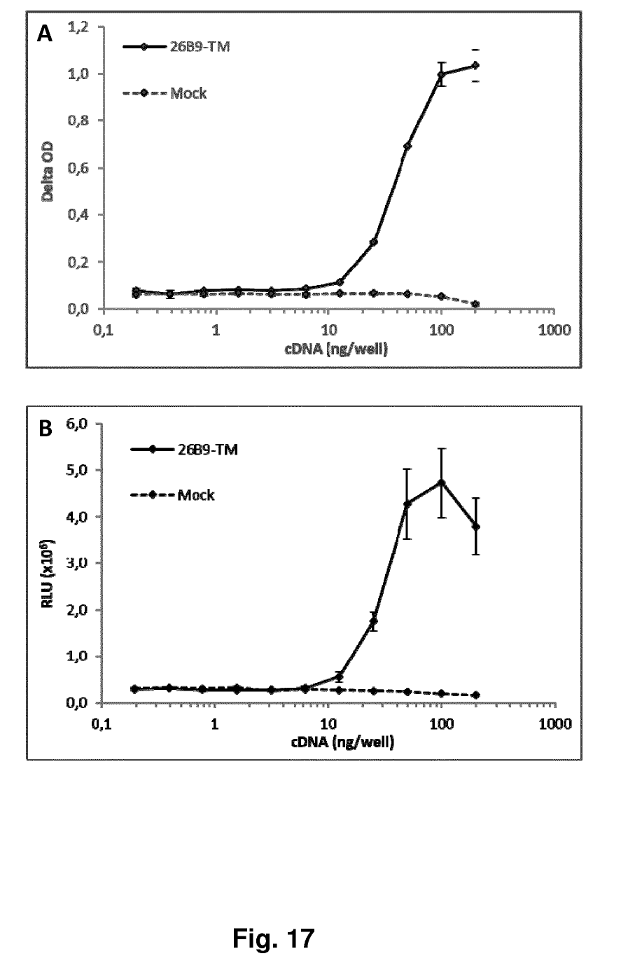

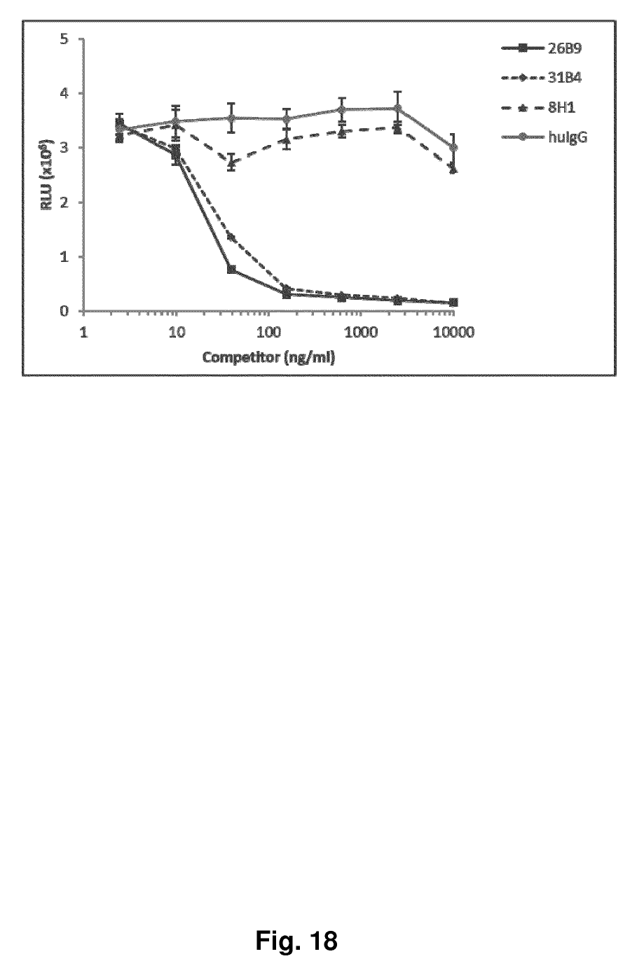

As illustrated in FIG. 7A, in accordance with the novel method of the present invention the ligand of interest is labeled and provided as a fusion protein comprising a reporter, i.e. luciferase fusion protein such as Gaussia luciferase. Cellular binding of the ligand is measured by cell-associated reporter, i.e. luciferase activity. This assay permits the assessment whether antibodies directed against the ligand prevent its ability to bind to the respective receptor on the cells (FIGS. 7G and 7H).

TABLE 6
List of recombinant proteins used in the neutralization
assays (all proteins).

| Protein | Provider | Cat. No. |
|---|---|---|
| rhIL-20 | R&D Systems | 1102-IL/CF |
| rmIL-20 | R&D Systems | 1204-ML/CF |
| rhIL-22 | ImmunoTools | 11340227 |
| rhIFNA2b | ImmunoTools | 11343516 |
| rhIL-32γ | R&D Systems | 4690-IL/CF |
| g1 IFNA2, A4, A5, | fusion proteins of human IFNs with | |
| A6, A7, A8, A10, | Gaussia luciferase | |
| A14, A16, A17, | | |
| A21, B, W | | |
| g2 IL-22 | fusion protein of human IL-22 with | |
| | Gaussia luciferase | |
| g1 IL-20 | fusion protein of human IL-20 with | |
| | Gaussia luciferase | |
| g1 IFNW | fusion protein of human IFNW with | |
| | Gaussia luciferase | |

First, human-derived anti-IFN-alpha monoclonal antibodies have been isolated and proved in the Luminescent Cell Binding Assay (LCBA) to be suitable in receptor binding and neutralizing fusion proteins of human IFNs with Gaussia luciferase. For a detailed description of exemplary anti-IFN-alpha antibodies and IFNA subtypes and molecules see applicant's co-pending international application WO2015/001013, the disclosure content which is incorporated herein by reference, in particular Examples 1 to 9, Table 1 and FIG. 1 disclosing the nucleotide and amino acid sequences of the variable and constant regions (VH, VL, CH, CL) regions of IgG1, kappa, IFNA, e.g. IFNA2, -4 and -14 recognizing antibodies 5D1, 13B11, 19D11, 25C3, and 31B4 as well of antibodies 26B9 and 8H1 which besides IFNA subtypes effectively recognize IFNW (interferon omega) as well as their functional characterization including sources of IFNA subtype molecules. For primer sequences used for cloning the Gaussia-luciferase-IFN-fusion and Gaussia-luciferase-IL-22-fusion constructs see applicant's international application WO 2013/098419 published on Jul. 4, 2013, the disclosure content which is incorporated herein by reference.

### Interferon(s)-Gaussia Luciferase

30,000 HEK 293T MSR cells were seeded in white half area 96-well tissue culture plates (Cat. No. 3688, Corning Inc.). The following day, supernatants of HEK 293T cells transiently expressing human IFN-Gaussia luciferase fusion proteins were mixed with anti-IFN mAbs, control IgG or excess concentrations of unlabeled recombinant IFNA2 and pre-incubated for one hour at 37° C. After pre-incubation, the mixtures were used to stimulate HEK 293T MSR cells for 40 minutes at 37° C. Upon binding, cells were washed three times with PBS, and the Gaussia luciferase assay was developed using the Gaussia Flash Assay Kit according to the manufacturer's instructions (Cat. No. 16159, Thermo Fisher Scientific).

The results of this experiment shown in FIG. 7G demonstrate that binding of IFNA5-Gaussia luciferase-fusion protein (g1 IFNA5) to HEK 293T MSR cells is inhibited by unlabeled recombinant human (rh) IFNA2 (3 μg/ml) and by exemplary human-derived monoclonal IFN antibody 19D11 (1.7 μg/ml). Furthermore, binding of g1 IFNA2, A4, A5, A6, A7, A8, A10, A14, A16, A11 and A21 fusion proteins to HEK 293T MSR cells is inhibited by exemplary human-derived monoclonal IFN antibody 19D11. In contrast, binding of g1 IFNB and W is unaffected by 19D11 and binding of all g1 IFNs is unaffected by a control human antibody (huIgG) (FIG. 7H). Antibody concentration: 5 μg/ml.

Second, human-derived IL-22 monoclonal antibodies have been isolated and proved in the Luminescent Cell Binding Assay (LCBA) to be suitable in receptor binding and neutralizing fusion proteins of human IL-22 with Gaussia luciferase. For a detailed description of exemplary anti-IL-22 antibodies and molecules see applicant's international application WO 2013/098419, the disclosure content which is incorporated herein by reference.

### IL-22-Gaussia Luciferase

COLO 205 colorectal carcinoma cells were detached with Versene according to the manufacturer's instructions (Cat. No. 15040-066, Invitrogen) and washed twice with PBS. 8×105 cells were transferred into V-bottom 96-well plates (Cat. No. 24957, Thermo Fisher Scientific) which had been blocked for one hour with 200 μl COLO 205 complete culture medium per well (RPMI 1640 GlutaMAX, 10% Foetal Bovine Serum, Cat. No. 61870-044 and 10109155, respectively, Invitrogen). Supernatants of HEK 293T cells transiently expressing human IL-22-Gaussia luciferase fusion proteins were mixed with anti-IL-22 mAbs, control IgG or excess concentrations of unlabeled recombinant IL-22 and pre-incubated for one hour at 37° C. After pre-incubation, the mixtures were used to stimulate COLO 205 cells for one hour at 37° C. in V-bottom 96-well plates. Upon binding, cells were washed three times with PBS, and the Gaussia luciferase assay was developed using the Gaussia Flash Assay Kit according to the manufacturer's instructions (Cat. No. 16159, Thermo Fisher Scientific). Cells were either lysed or not lysed prior to addition of the luciferase substrate.

Human IL-22-Gaussia luciferase fusion proteins bind specifically to COLO 205 cells. Cells were incubated with supernatants of HEK 293T cells expressing IL-22-Gaussia luciferase fusion proteins (g2 IL-22) in the absence of inhibitors (−) or in the presence of competitive inhibitors as indicated. The results of this experiments shown in FIG. 14A demonstrate that binding of g2 IL-22 to COLO 205 cells is inhibited by unlabeled rhIL-22 and not by the unrelated rhIFNA2. COLO 205 cells were incubated with g2 IL-22-containing supernatants of HEK 293T cells in the absence of inhibitors or in the presence of unlabeled rhIL-22 or rhIFNA2 (1 μg/ml each) as indicated. Cells were either lysed or not lysed before light output was recorded. FIG. 14B demonstrates that binding of g2 IL-22 to COLO 205 cells is inhibited in a dose-dependent manner by exemplary human-derived IL-22 monoclonal antibody 30G1. Binding is unaffected by a control human antibody (huIgG).

Third, since the LIPS assay has not been predictive for the antagonizing, i.e. neutralizing activity of the candidate antibodies, a new assay for the assessment of the binding of a ligand of interest to cells expressing the relevant receptor(s) has been developed in accordance with the present invention for use in determining whether candidate antibodies directed against the ligand prevent its ability to bind to the respective receptor(s) on the cells.

For this reason, first the phospho-STAT3 assay described in Example 3 has been applied in order to investigate whether both the receptor(s), i.e. Type I and Type II IL-20 receptors as well as the ligand labeled with a reporter, i.e. IL-20-Gaussia luciferase fusion protein are biologically active in the assays. As shown in FIGS. 7C and 7D, the IL-20-Gaussia luciferase fusion protein (g1 IL-20) is functionally active. Cells were incubated with a serial dilution of g1 IL-20 supernatants or with control medium (NC). Detection of total and phosphorylated STAT3, IL-20RB-Myc-DDK, IL-20RA-Myc-DDK and IL-22RA (RA subunits) in Western blots (FIG. 7A). As a control it is shown that the IL-20 fusion protein specifically binds to HEK 293T MSR cells expressing both subunits of 11-20 receptor Type I (FIG. 7D). Binding is abrogated by unlabeled rhIL-20 (3 μg/ml). Thus, supernatants of HEK 293T containing IL-20-Gaussia luciferase fusion proteins specifically activate STAT3 in HEK 293T MSR cells transiently expressing Type I and Type II IL-20 receptors.

Accordingly, the binding characteristics of the ligand and ligand-binding domain should substantially resemble those in vivo for which reason it is prudent to expect that activators and antagonists identified and obtained with the assay of the present invention are also biologically active in vivo.

As next step, candidate antibodies were subjected to the chemiluminescent cellular binding assay for assessing their neutralizing activity.

### Chemiluminescent Cellular Binding Assay

For the IL-20 chemiluminescent cellular binding assay, 10,000 HEK 293T MSR cells were seeded in white half area 96-well tissue culture plates (Cat. No. 3688, Corning Inc.). The following day, cells were co-transfected with either IL20RA-Myc-DDK and IL20RB-Myc-DDK (Type I IL-20 receptor; Cat. No. RC212546 and RC213197, OriGene, Rockville, Md., USA) or IL22RA (Cat. No. SC322566, OriGene) and IL20RB-Myc-DDK expression plasmids (Type II IL-20 receptor). The day following transfection, supernatants of HEK 293T cells transiently expressing human IL-20-Gaussia luciferase fusion proteins were mixed with anti-IL-20 mAbs, control IgG or excess concentrations of unlabeled recombinant IL-20 and pre-incubated for one hour at 37° C. After pre-incubation, the mixtures were used to stimulate HEK 293T MSR cells transiently expressing IL-20 receptors for 30 minutes at 37° C. Upon binding, cells were washed three times with PBS, and the Gaussia luciferase assay was developed using the Gaussia Flash Assay Kit according to the manufacturer's instructions (Cat. No. 16159, Thermo Fisher Scientific).

As shown in FIGS. 7E and 7F the observed effects in the cellular binding assay closely reflect the levels of neutralizing activity observed for the candidate antibodies in Examples 3 and 4 as well as FIGS. 4 to 6. As a control, binding of g1 IL-20 to HEK 293T MSR cells expressing Type I IL-20 receptors is inhibited in a dose-dependent manner by rhIL-20, rmIL-20 and exemplary human-derived IL-20 monoclonal antibody 20A10 (FIG. 7E). Binding is unaffected by the unrelated rhIL-32γ and a control human IgG (huIgG). Exemplary anti-IL-20 antibodies 2A11, 20A10 and 7D1 neutralize binding of g1 IL-20 to HEK 293T MSR cells transiently expressing Type I and Type II IL-20 receptors. Unlabeled rhIL-20 (1 μg/ml) serves as a positive control. Antibody concentration: 5 μg/ml (FIG. 7F). In line with the results of phospho-STAT3 assay (Example 3) and the KZ136-NLuc reporter neutralization assay (Example 4), anti-IL-20 antibody 20A10 showed the highest inhibitory, i.e. neutralization activity, whereas MAbs 2A11 and 7D1 only showed a slightly weaker neutralization capacity of g1 IL-20 binding to both Type I and Type II IL-20 receptors. In contrast, anti-IL-20 antibody 6E11 revealed modest neutralization capacity of g1 IL-20 binding to Type I but lack of neutralization capacity towards Type II IL-20 receptors. No or much reduced neutralization capacity could be observed in respect to MAb 6H2.

Thus, the novel cellular ligand receptor binding assay of the present invention provides a reliable means for predicting the biological, i.e. neutralizing activity of candidate compounds, in particular anti-ligand antibodies which significantly simplifies identification and obtaining therapeutically useful antibodies which otherwise might have not been further pursued or identified in the first place.

Indeed, further IC 50 (half minimal (50%) inhibitory concentration) analysis of human-derived anti-IL-20 monoclonal antibodies in the chemiluminescent cellular binding assay demonstrated effective neutralization capacity of IL-20 by exemplary IL-20 antibodies 20A10, 7D1 and 2A11 (FIG. 8). To this end, HEK 293T MSR cells transiently expressing Type I or Type II IL-20 receptors were incubated with g1 IL-20 supernatants in the presence of human-derived IL-20 monoclonal antibodies as indicated, FIG. 8 A: 20A10, B: 7D1 and C: 2A11. FIG. 8D summarizes IC 50 values of exemplary antibodies 20A10, 7D1 and 2A11, supporting the data obtained by the phospho-STAT3 assay (Example 3) and the KZ136-NLuc reporter neutralization assay (Example 4) that anti-IL-20 antibody 20A10 showed the highest neutralization activity. MAb 2A11 only showed a slightly weaker neutralization capacity as 20A10. In contrast, MAb 7D1 seems to need the highest IC 50 to inhibit g1 IL-20 binding to Type I or Type II IL-20 receptors.

### Example 6: Cross-Competition with Human-Mouse Chimeric Constructs and Cross-Reactivity of Anti-IL-20 Antibodies 20A10, 2A11, 7D1 and 6E11

In order to determine the number of different binding sites, differential binding of anti-IL-20 MABs to distinct antigen binding sites is examined. For this purpose MABs are expressed either with human (hMAB) or mouse (hmMAB) Fc and cross-competition experiments are carried out by GLuc sandwich ELISA. GLuc-Il20 is captured by a pre-coated rabbit anti-GLuc antibody. Binding of hmMABs in the presence of large excess of human MABs is detected by a HRP-conjugated secondary antibody directed against the Fc portion of the primary antibody (see scheme of the experimental setup in FIG. 9A).

### Method:

96 well microplates (Costar, USA) were coated with rabbit anti-GLuc antibody (NEB, E8023S) diluted 1/250 in PBS, 30 μl/well over night at 4° C. Plates were washed with PBS-T and blocked 1 h at room temperature with PBS containing 2% BSA (Sigma, Buchs, Switzerland). 30 μl GLuc-IL-20 was added at a final concentration of 2×10⁶ LU/well and incubated for 2 h at room temperature. The chimeric antibodies were premixed with the human competitor antibodies at a final concentration of 0.5 μg/ml vs. 3 μg/ml in 30 μl PBS and added to the plates. Upon incubation for 2 h at room temperature the plates were washed with PBS-T and the binding of the human-mouse chimeric antibodies was determined using a horseradish peroxidase conjugated goat anti-mouse IgG Fc-gamma specific antibody (Jackson Immuno Research, 1:500 in 0.5% BSA-PBS) followed by measurement of the HRP activity using a TMB substrate solution (Sigma, Buchs, Switzerland).

As can be seen from FIGS. 9B-F and Table 7 below, chimeric anti-IL-20 antibodies 20A10 (hm), 2A11 (hm) and 6E11 (hm) compete with their cognate human counterpart only, indicating that each of the human-derived anti-IL-20 antibodies 20A10, 2A11 and 6E11 recognize different binding site(s) of IL-20. In contrast, chimeric anti-IL-20 antibody 7D1 (hm) does not only compete with its human counterpart but also with human-derived IL-20 antibody 6H2, indicating a possible partially overlapping epitope of these antibodies.

However, in view of the functional assay in Examples 3-5 and shown in FIGS. 4B, 7F and 8D, antibody 7D1 does seem to effectively neutralize IL-20 activity. Therefore, it is assumed that the epitopes of antibody 7D1 and 6H2 are not identical but overlap or that the antibodies interfere with binding to IL-20 due to steric hindrance.

In view of the experiments performed in accordance with the present invention, demonstrating inter alia the lack of relationship between binding affinity/specificity and neutralizing activity it is clear that the novel cellular ligand-binding assay of the present invention is useful and essential in selecting and providing anti-IL-20 antibodies for therapeutic use. Nevertheless, also IL-20 antibodies of the present invention that do not seem to effectively neutralize IL-20 activity are useful, for example in diagnostic applications.

TABLE 7
Results of the cross-competition experiments of exemplary antibodies of the present
invention. Human MAB (hMAB) were added in large excess to plates coated with the
respective antigens before addition of MABs with mouse Fc (hmMAB).

| | | Human MAB competitor | | | | |
|---|---|---|---|---|---|---|
| g2 IL-20 | | 2A11 | 6E11 | 6H2 | 7D1 | 20A10 |
| hm-MAB | 2A11 | +++++ | — | — | — | — |
| | 6E11 | — | +++++ | — | — | — |
| | 6H2 | — | — | +++++ | +++++ | — |
| | 7D1 | — | — | +++++ | +++++ | — |
| | 20A10 | — | — | — | — | +++++ |

Additionally, the binding of exemplary human-derived anti-IL-20 monoclonal antibodies 6E11, 2A11 and 20A10 of the present invention to mouse and human IL-20 was investigated to determine and compare cross-reactivity against mouse and human IL-20 epitopes by GLuc-Sandwich ELISA (see scheme of the experimental setup in FIG. 10A).

### Method:

96 well microplates (Costar, USA) were coated with rabbit anti-GLuc antibody (NEB, E8023S) diluted 1/250 in PBS, 30 μl/well over night at 4° C. Plates were washed with PBS-T and blocked for 1 h at room temperature with PBS containing 2% BSA (Sigma, Buchs, Switzerland). 30 μl GLuc-IL-20 was added at a final concentration of 2×10⁶ LU/well and incubated for 2 h at room temperature. The competitor antigens recombinant human IL-20 and recombinant mouse IL-20 were titrated to the antibodies to be tested (fixed concentration of 1 μg/ml) in a serial dilution ranging from 10 μg/ml to 4.6 ng/ml. 30 μl per well of the mixtures were incubated in the wells for 1.5 h at room temperature. Plates were washed with PBS-T and the binding of human IgG to the antigen of interest in presence of the competitors was determined using a horseradish peroxidase conjugated goat anti-human IgG Fc-gamma-specific antibody (Jackson ImmunoResearch, Europe Ltd., Cambridgeshire, UK), followed by measurement of the HRP activity using a TMB substrate solution (TMB, Sigma, Buchs, Switzerland).

As shown in FIGS. 10B-D human IL-20 can inhibit binding of all tested anti-IL-20 antibodies to g2 IL-20. Murine IL-20 can inhibit binding of all tested anti-IL-20 antibodies, except 6E11, to g2 IL-20. Therefore, exemplary anti-IL-20 antibodies 2A11 and 20A10 show species cross-reactivity, whereas MAb 6E11 only recognizes hIL-20. However, in view of the functional assay in Examples 3-5 and shown in FIG. 4B, antibody 2A11 is not as effective as MAb 20A10 in neutralizing mIL-20. 2A11 is a strong binder, but apparently recognizes a region of IL-20 that is not involved in the interaction between IL-20 and its receptors. 2A11 and 7D1 weakly neutralize rhIL-20, but show better neutralization of GLuc IL-20. This result further demonstrates inter alia the lack of relationship between binding affinity/specificity and neutralizing activity. It is clear that the novel cellular ligand-binding assay of the present invention is useful and essential in selecting and providing anti-IL-20 antibodies for therapeutic use. Nevertheless, also IL-20 antibodies of the present invention that do not seem to effectively neutralize IL-20 activity are useful, for example in diagnostic applications.

### Example 7: Validation of Subject Antibodies

Antibodies provided by the present invention are tested in concern of their neutralizing activity towards human IL-20 in animal disease models. When performing such experiments it has to be ensured that human IL-20 induces diseased phenotypes in mice and that no cross-reaction occurs between the tested IL-20 antibodies of the present invention and the murine IL-20 homologues. Since no adequate model system for IL-20 was available in the prior art, the present Example describes and provides such a system to test IL-20 neutralizing antibodies that do not cross react with mouse IL-20.

### Ear Inflammation Assay

Ear inflammation phenotype was induced in 8 weeks old C57BL/6J (WT; from Charles River) mice by intradermal injection of human cytokine IL-20 or PBS control into each ear given on alternate days at Day 1, Day 3, Day 6 and Day 8 (20 μl/ear, 1000 ng/ear, 2000 ng/mouse/day) using a 30-gauge needle. Treatment with the exemplary anti-IL-20 2A11, 7D1 and 20A10 antibodies of the present invention were tested on these animals in respect of their neutralizing potential to reduce the induced ear inflammation phenotype. Two IP injections of 2A11, 7D1 and 20A10 or control human IgG [200 μg,] were administered to the animals at day 0 and day 6. The mice were sacrificed at day 10.

To test a potential therapeutic effect of the antibodies of the present invention ear thickness measurements of the animals were taken with a Mitutoyo digital micrometer during the IL-20 administration by daily measurements prior to IL-20 injection. Furthermore, body weight has been monitored during the treatment, however, no significant weight changes have been observed in any of the animal groups due to the treatment applied. In addition, after sacrifice of the animals H&E (hematoxylin and eosin; see Harris, H. F., J. Appl. Microscopy III (1900), 777-781 and Mallory, F. B.: Pathological technique. Philadelphia, Saunders, (1938)) histology stainings of the ears are performed. These experiments show that the induction of ear swelling with intradermal injection of human IL-20 is reduced in the presence of 2A11, 7D1 and 20A10 neutralizing antibody; see FIG. 11. This is significant from Day 6 onwards to varying degrees of statistical significance. Exemplary anti-IL-20 antibodies 2A11, 7D1 and 20A10 are able to neutralize the injected IL-20 in a mouse model. Accordingly, the data presented herein indicates that the anti-IL-20 antibodies of the present invention are effective against IL-20 in cytokine induced ear inflammation experiments, demonstrating the therapeutic value of the IL-20 specific binding molecules of the present invention.

### Example 8: Antibody Affinity Measurements Using Surface Plasmon Resonance (SPR) Technology

For affinity determination of the antibodies of the present invention surface plasmon resonance SPR measurements were performed using a Biacore T200 SPR instrument (GE Healthcare) according to the manufacturer's instructions. CM5 chips were coupled with an anti-huIgG Fc mouse IgG1 mAb (GE Healthcare) and the anti-IL20 antibodies were captured to saturation. As analytes human and mouse IL20 were applied in repeated runs in concentrations from 1.5 to 100 nM. On- and off-rates and the resulting KD were determined by the instruments analysis software using a 1:1 binding fit. (FIG. 12)

### Example 9: LCBA Assay Using Transmembrane Version of a Subject Antibody as the Ligand Bind Domain

Constructs, sequence information and references to Platelet-derived growth factor receptor (PDGFR)-transmembrane (TM) mediated surface expression of subject antibody 26B9 are depicted in FIGS. 15 and 16; see also the Figure legends thereto, supra, for further details.

### Ligand-Binding to Transmembrane Antibodies

30,000 HEK 293T MSR cells were seeded in white half area 96-well tissue culture plates (Cat. No. 3688, Corning Inc.). During seeding, cells were transfected with 100 ng cDNA encoding a transmembrane version of anti-IFN mAb 26B9 (26B9-TM) (SEQ ID: 67, Ho et al., Proc. Natl. Acad. Sci. 103 (2006), 9637-9642; Zhou et al., Mabs 2 (2010), 508-518) using Fugene HD (Cat. No. E2311, Promega, Madison, Wis., USA) (FIG. 17, 18). Surface antibody (26B9-TM) expression was analyzed 48 hours after transfection in a cell-based ELISA (FIG. 17A). Forty-eight hours following transfection, supernatants of HEK 293T cells transiently expressing human IFNW-Gaussia luciferase fusion proteins (g1 IFNW) were used to stimulate the previously transfected HEK 293T MSR cells for 40 minutes at 37° C. Alternatively, the g1 IFNW supernatants were mixed with anti-IFN mAbs or control IgG and preincubated for one hour at 37° C. After preincubation, the mixtures were used to stimulate HEK 293T MSR cells transiently expressing 26B9-TM for 40 minutes at 37° C. Upon binding, cells were washed three times with PBS, and the Gaussia luciferase assay was developed using the Gaussia Flash Assay Kit according to the manufacturer's instructions (Cat. No. 16159, Thermo Fisher Scientific) showing that g1 IFNW was specifically binding to cells expressing 26B9-TM (FIG. 17B). PDGFR-mediated surface expression of monomeric Fv and further references of the PDGFR-domain are described in Ho et al. Proc. Natl. Acad. Sci. 103 (2006), 9637-9642. PDGFR-mediated surface expression of complete IgGs are described in Zhou et al. Mabs 2 (2010), 508-518.

### Cross-Competition Assay of Anti-IFNW Antibodies.

Above experimental setup has been also used to test cross-competition between exemplary antibodies 26B9, 31B4 and 8H1 of the present invention (see FIG. 18). HEK 293T MSR cells were reverse-transfected with cDNA encoding 26B9-TM. Forty-eight hours after transfection, g1 IFNW was mixed and preincubated for one hour with soluble anti-IFNW antibodies 26B9, 31B4, 8H1 or a control IgG (huIgG). Following incubation, the mixtures were added to the transfected cells and binding was analyzed in the chemiluminescent cellular binding assay. As shown in FIG. 18 binding of g1 IFNW to 26B9-TM is competed dose-dependently by soluble 26B9 and by the clonally related 31B4 antibody. In contrast, binding is not affected by a control IgG or by exemplary anti-IFNW antibody 8H1. These results indicate that exemplary antibodies 26B9 and 31B4 share similar epitopes, while 8H1 appears to bind to a distinct epitope.

### Example 10: Antigen Mapping of the Anti-IL-20 Monoclonal Antibodies

Binding regions of MABs to their respective antigens can be mapped by, e.g., PepStar™ analysis. Therefore, overlapping 18mer peptides (14 amino acid overlap) were designed to cover humane and murine IL-20, respectively, including all known variants. The peptides (FIG. 13B-human IL-21 and FIG. 13C—murine 11-20) and full-length antigen (as positive control; FIG. 13A) are spotted on microarray and the peptide microarray is incubated with the primary antibody followed by a fluorescently labelled secondary antibody directed against the Fc portion of the primary antibody. To avoid false negatives caused by steric hindrance, an optimized hydrophilic linker moiety is inserted between the glass surface and the antigen derived peptide sequence.

Furthermore, a set of 17 peptides comprising the sequence 101-PDHYTLRKISSLANSFL-117 (SEQ ID No: 72) of human IL-20 were synthesized for epitope characterization by alanine-scan. Here, each individual amino acid is separately substituted by alanine to assess its contribution to the antibody binding.

The samples were printed onto the microarray slides with a concentration of 1 μg/ml. Microarrays were subsequently incubated with the antibodies of the present invention in blocking buffer for 60 min at 30° C. For detection an Cy5-anti-human IgG (JIR 209-175-082) secondary antibody was used at a concentration of 1 μg/ml diluted in blocking buffer and incubated for 60 min at 30° C. Additionally, an incubation with the fluorescently labeled secondary antibody only was performed as a control experiment to detect potentially false positive signals. Before each step, microarrays were washed with washing buffer. For analysis, the signal intensities were mapped on the protein sequences to allow identification of linear epitopes.

### Buffers and Solutions

50 mM TBS-buffer including 0.1% Tween20 (JPT), pH 7.2
3 mM SSC-buffer (JPT), pH 7.0
Blocking buffer (Pierce International, Superblock TBS T20, order#37536)

### Hardware and Software

Peptide microarrays (JPT Peptide Technologies GmbH, Berlin, Germany; batch #2611)
Axon Genepix Scanner 4200AL
Spot-recognition software GenePix

Such peptide mapping has been performed for exemplary antibodies 20A10, 2A11, 6E11 and 7D1 of the present invention on peptide arrays of 18mer peptides of human and murine IL-20, respectively. The results of the assays are shown in FIG. 13. Exemplary antibody 20A10 binds specifically to peptide 20 (PDHYTLRKISSLANSFLT SEQ ID NO: 69) of IL-20 (FIG. 13D) however it does not bind to peptide 19 consisting of the amino acid sequence NYQTPDHYTLRKISSLAN (SEQ ID NO: 71). Thus, the binding specificity of antibody 20A10 is different from that of anti-IL20 antibodies 15D2 and 5B7 described in international application WO2010/000721, which bind both to peptides 19 and 20 of IL-20 (designated in this application as peptides 25 and 26 for antibody 5B7 and peptides 86 and 87 for antibody 15D2; see WO 2010/000721 in FIGS. 6A, 6B, 7A and 7B).

Exemplary antibody 2A11 binds to murine IL-20 in cross competition assay (FIG. 10C) but not on microarray (FIG. 13A), while exemplary anti-IL-20 antibody 20A10 of the present invention binds in both cases to the murine and human IL-20 (FIG. 10D and FIG. 13A). As shown by the alanine-scan (FIG. 13G) several residues within the sequence of the peptide 20 are crucial for interacting with the exemplary antibody 20A10, binding of which is abolished or at least severely reduced if these crucial amino acids are substituted.

The importance of the particular amino acids for binding of the exemplary antibody 20A10 is summarized in the following textural transcription of the 15aa epitope bin: 101-PDHYTLRKISSLANSFLT-118, wherein the amino acids crucial for binding by exemplary antibody 20A10 are indicated in bold, italic and underlined, interacting and less crucial amino acids by underline. In contrast, in the terminal deletion and alanine scan epitope mapping for antibodies 5B7 and 15D2 described in WO 2010/000721 suggest a smaller linear epitope of 12 amino acids form H103-N114 in which however only three amino acid positions (H103, S111 and N114) were found to be invariant and crucial for binding by antibodies 5B7 and 15D2; see alanine-scan data in WO 2010/000721 in Example 7 and 8 as well as in FIGS. 7 and 8.

A further interesting observation is that while antibody 20A10 binds to full-length human and murine IL-20 (FIGS. 10D, 12C, and in context of the microarray FIG. 13A) it does not bind in the array antigen mapping assay to a corresponding peptide derived from mouse IL-20, which differs in its amino acid sequence from the human IL-20 101-118-peptide at positions Y104H and T118I only, wherein the latter seems to be dispensable for 20A10 binding, nor to any other mouse IL-20 derived 18mer peptide (FIG. 13C). Accordingly, antibody 20A10 might recognize a conformational epitope of human and mouse IL-20, which can also be formed or mimicked by the human IL-20 derived peptide 101-PDHYTLRKISSLANSFLT-118 (SEQ ID NO: 69), or wherein the amino acid sequence of this peptide represents the main constituent of this conformational epitope. Hence, the large epitope bin of antibody 20A10 and its lack of binding to peptides derived from mouse IL-20 makes this antibody highly specific for IL-20 for which reason cross-reaction with other cytokines, e.g., IL-10 and IL-10-like cytokines is unlikely.

1. A human-derived monoclonal anti-human interleukin-20 (IL-20) antibody or an IL-20 binding fragment, synthetic or biotechnological derivative thereof, comprising in its variable region:
(a) at least one complementarity determining region (CDR) of the V_{H} and/or V_{L} variable region comprising the amino acid sequences selected from the group consisting of
(i) (V_{H}) (SEQ ID NOs: 2, 10, 18 and 26); and
(ii) (V_{L}) (SEQ ID NOs: 4, 12, 20 and 28);
(b) an amino acid sequence of the V_{H} and/or V_{L} region as depicted in FIG. 1;
(c) at least one CDR consisting of an amino acid sequence resulting from a partial alteration of any one of the amino acid sequences of (a); or
(d) a heavy variable chain and/or light variable chain region comprising an amino acid sequence resulting from a partial alteration of the amino acid sequence of (b).

2. The antibody or IL-20 binding fragment of claim 1, which reduces or neutralizes a biological activity of IL-20.

3. The antibody or IL-20 binding fragment, synthetic or biotechnological derivative of claim 1, which recognizes an IL-20 derived peptide consisting of the amino acid sequence PDHYTLRKISSLANSFLT (SEQ ID NO: 69), DHYTLRKISSLANSF (SEQ ID NO: 70) or PDHYTLRKISSLANSFL-(SEQ ID No: 72), wherein P101, 1109, S110 and/or L117 may be substituted by another amino acid, and which does not or does not substantially recognize a peptide consisting of the amino acid sequence NYQTPDHYTLRKISSLAN (SEQ ID NO: 71).

4. The antibody or IL-20 binding fragment of claim 1, which does not substantially bind and/or neutralize murine IL-20.

5. The antibody or IL-20 binding fragment of claim 2, wherein the biological activity is at least one of:
(a) human IL-20 signaling in a cell based STAT (signal transducers and activators of transcription) activation assay;
(b) inhibition of IL-20 cytokine cell-surface receptor binding;
(c) human IL-20 mediated activation of human IL-20 R Type I/Type II receptor complexes; and
(d) pro-inflammatory activity of human IL-20.

6. The antibody or IL-20 binding fragment of claim 1, wherein the CDRs are at least 90% identical to the corresponding CDR indicated in FIG. 1 and/or the amino acid sequence of the framework region of the VH and/or VL variable region is at least 90% identical to the corresponding framework region indicated in FIG. 1.

7. The antibody or IL-20 binding fragment of of claim 1, which is an immunoglobulin of the IgG1 or IgG4 subclass.

8. The antibody or IL-20 binding fragment thereof of claim 1, comprising a C_{H} and/or C_{L} constant region comprising an amino acid sequence selected from the C_{H} and C_{L} amino acid sequences of SEQ ID NOs.: 6, 14, 22 or 30 or an amino acid sequence with at least 95% identity.

9. An antibody or antigen-binding molecule which competes with an antibody of claim 1 for binding to human IL-20.

10. The antibody of claim 1, which is selected from the group consisting of a single chain Fv fragment (scFv), an F(ab′) fragment, an F(ab) fragment, and an F(ab)₂ fragment.

11. One or more polynucleotide(s) encoding at least the variable region of one immunoglobulin chain of the antibody or antigen-binding fragment of claim 1.

12. One or more vector(s) comprising the polynucleotide(s) of claim 11.

13. A host cell comprising said one or more vector(s) of claim 12.

14. A method of producing an anti-human IL-20 antibody or IL-20 binding fragment thereof comprising culturing the host cell of claim 13 and isolating the anti-human IL-20 antibody or IL-20 binding fragment thereof from the culture.

15. An immunoconjugate comprising the anti-IL-20 antibody or IL-20 binding fragment of claim 1, which comprises a radionuclide, enzyme, substrate, cofactor, fluorescent marker, chemiluminescent marker, peptide tag, heavy metal, magnetic particle, drug, or a toxin.

16. A composition comprising the anti-IL-20 antibody or IL-20 binding fragment of claim 1, wherein the composition is
(a) a pharmaceutical composition and further comprises a pharmaceutically acceptable carrier and optionally further comprises an additional agent useful for treating an inflammatory disease; or
(b) a diagnostic composition or kit and further comprises reagents conventionally used in immuno- or nucleic acid based diagnostic methods.

17.-32. (canceled)
